SEQUENCE LISTING (82 sequences)

SEQ ID NO: 1 - DNA, 363 bp, synthetic construct
  misc_feature 1..363: note = Description of Artificial Sequence: Syntheticpolynucleotide
  source 1..363: mol_type = other DNA
caggtgcagctgcaggagtcgggcccaggactggtgaagccttcggagaccctgtccctcacctgcactgtctctggtgactccatcattactttctactggagctggatccggcagcccccagggaggggactggagtggattgggtatatctattacagtgggatcaccaactacaatccctccctcaagagtcgagtcaccatatcagtagacacgtccaagaaccaggtctccctgaaactgagttctgtgaccgctgcggacacggccgtgtattattgtgcgagagtgtccgaggattcctactttcactacgggatggacgtctggggccaagggaccacggtcaccgtctcctca

SEQ ID NO: 2 - protein, 121 aa, synthetic construct
  REGION 1..121: note = Description of Artificial Sequence: Syntheticpolypeptide
QVQLQESGPGLVKPSETLSLTCTVSGDSIITFYWSWIRQPPGRGLEWIGYIYYSGITNYNPSLKSRVTISVDTSKNQVSLKLSSVTAADTAVYYCARVSEDSYFHYGMDVWGQGTTVTVSS

SEQ ID NO: 3 - DNA, 24 bp, synthetic construct
  misc_feature 1..24: note = Description of Artificial Sequence: Syntheticoligonucleotide
  source 1..24: mol_type = other DNA
ggtgactccatcattactttctac

SEQ ID NO: 4 - protein, 8 aa, synthetic construct
  REGION 1..8: note = Description of Artificial Sequence: Syntheticpeptide
GDSIITFY

SEQ ID NO: 5 - DNA, 21 bp, synthetic construct
  misc_feature 1..21: note = Description of Artificial Sequence: Syntheticoligonucleotide
  source 1..21: mol_type = other DNA
atctattacagtgggatcacc

SEQ ID NO: 6 - protein, 7 aa, synthetic construct
  REGION 1..7: note = Description of Artificial Sequence: Syntheticpeptide
IYYSGIT

SEQ ID NO: 7 - DNA, 45 bp, synthetic construct
  misc_feature 1..45: note = Description of Artificial Sequence: Syntheticoligonucleotide
  source 1..45: mol_type = other DNA
gcgagagtgtccgaggattcctactttcactacgggatggacgtc

SEQ ID NO: 8 - protein, 15 aa, synthetic construct
  REGION 1..15: note = Description of Artificial Sequence: Syntheticpeptide
ARVSEDSYFHYGMDV

SEQ ID NO: 9 - DNA, 366 bp, synthetic construct
  misc_feature 1..366: note = Description of Artificial Sequence: Syntheticpolynucleotide
  source 1..366: mol_type = other DNA
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggatcacccactacaacccctccctcaagagtcgagtcaccatatcagtagacacgtccaagatccagttctccctgaagctgagttctgtgaccgctgcggacacggccgtgtattactgtgcgagatggggggttcggagggactactactactacggtatggacgtctggggccaagggaccacggtcaccgtctcctca

SEQ ID NO: 10 - protein, 122 aa, synthetic construct
  REGION 1..122: note = Description of Artificial Sequence: Syntheticpolypeptide
QVQLQESGPGLVKPSETLSLTCTVSGGSISSYYWSWIRQPPGKGLEWIGYIYYSGITHYNPSLKSRVTISVDTSKIQFSLKLSSVTAADTAVYYCARWGVRRDYYYYGMDVWGQGTTVTVSS

SEQ ID NO: 11 - DNA, 24 bp, synthetic construct
  misc_feature 1..24: note = Description of Artificial Sequence: Syntheticoligonucleotide
  source 1..24: mol_type = other DNA
ggtggctccatcagtagttactac

SEQ ID NO: 12 - protein, 8 aa, synthetic construct
  REGION 1..8: note = Description of Artificial Sequence: Syntheticpeptide
GGSISSYY

SEQ ID NO: 13 - DNA, 48 bp, synthetic construct
  misc_feature 1..48: note = Description of Artificial Sequence: Syntheticoligonucleotide
  source 1..48: mol_type = other DNA
gcgagatggggggttcggagggactactactactacggtatggacgtc

SEQ ID NO: 14 - protein, 16 aa, synthetic construct
  REGION 1..16: note = Description of Artificial Sequence: Syntheticpeptide
ARWGVRRDYYYYGMDV

SEQ ID NO: 15 - DNA, 324 bp, synthetic construct
  misc_feature 1..324: note = Description of Artificial Sequence: Syntheticpolynucleotide
  source 1..324: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 16 - protein, 108 aa, synthetic construct
  REGION 1..108: note = Description of Artificial Sequence: Syntheticpolypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 17 - DNA, 21 bp, synthetic construct
  misc_feature 1..21: note = Description of Artificial Sequence: Syntheticoligonucleotide
  source 1..21: mol_type = other DNA
cagtcagtctctagctcttat

SEQ ID NO: 18 - protein, 7 aa, synthetic construct
  REGION 1..7: note = Description of Artificial Sequence: Syntheticpeptide
QSVSSSY

SEQ ID NO: 19-20 - skipped (2 entries)

SEQ ID NO: 21 - DNA, 27 bp, synthetic construct
  misc_feature 1..27: note = Description of Artificial Sequence: Syntheticoligonucleotide
  source 1..27: mol_type = other DNA
caacagtacggaagcagcccgtggacg

SEQ ID NO: 22 - protein, 9 aa, synthetic construct
  REGION 1..9: note = Description of Artificial Sequence: Syntheticpeptide
QQYGSSPWT

SEQ ID NO: 23 - DNA, 1344 bp, synthetic construct
  misc_feature 1..1344: note = Description of Artificial Sequence: Syntheticpolynucleotide
  source 1..1344: mol_type = other DNA
caggtgcagctgcaggagtcgggcccaggactggtgaagccttcggagaccctgtccctcacctgcactgtctctggtgactccatcattactttctactggagctggatccggcagcccccagggaggggactggagtggattgggtatatctattacagtgggatcaccaactacaatccctccctcaagagtcgagtcaccatatcagtagacacgtccaagaaccaggtctccctgaaactgagttctgtgaccgctgcggacacggccgtgtattattgtgcgagagtgtccgaggattcctactttcactacgggatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 24 - protein, 447 aa, synthetic construct
  REGION 1..447: note = Description of Artificial Sequence: Syntheticpolypeptide
QVQLQESGPGLVKPSETLSLTCTVSGDSIITFYWSWIRQPPGRGLEWIGYIYYSGITNYNPSLKSRVTISVDTSKNQVSLKLSSVTAADTAVYYCARVSEDSYFHYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - DNA, 1347 bp, synthetic construct
  misc_feature 1..1347: note = Description of Artificial Sequence: Syntheticpolynucleotide
  source 1..1347: mol_type = other DNA
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggatcacccactacaacccctccctcaagagtcgagtcaccatatcagtagacacgtccaagatccagttctccctgaagctgagttctgtgaccgctgcggacacggccgtgtattactgtgcgagatggggggttcggagggactactactactacggtatggacgtctggggccaagggaccacggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 26 - protein, 448 aa, synthetic construct
  REGION 1..448: note = Description of Artificial Sequence: Syntheticpolypeptide
QVQLQESGPGLVKPSETLSLTCTVSGGSISSYYWSWIRQPPGKGLEWIGYIYYSGITHYNPSLKSRVTISVDTSKIQFSLKLSSVTAADTAVYYCARWGVRRDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 27 - DNA, 648 bp, synthetic construct
  misc_feature 1..648: note = Description of Artificial Sequence: Syntheticpolynucleotide
  source 1..648: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 28 - protein, 215 aa, synthetic construct
  REGION 1..215: note = Description of Artificial Sequence: Syntheticpolypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 366 bp, synthetic construct
  misc_feature 1..366: note = Description of Artificial Sequence: Syntheticpolynucleotide
  source 1..366: mol_type = other DNA
gaggtgcagctggtggagtctgggggaggcttggtccggcctggggggtccctgagactctcctgtacagcctctggattcaccttcagtacctttattatgttctgggtccgccaggctccagggaagggactggaatatgtttcatccattagtagtaatgggggtaccatatattatgcagactctgtgaagggcagattcaccatctccagagacaattccaagaacactctatatcttcaaatgggcagcctgagagctgaggacatggctgtatattattgtacgagagggggcgatttttggagtggttattatccttttgactactggggccagggaaccctggtcaccgtctcctca

SEQ ID NO: 30 - protein, 122 aa, synthetic construct
  REGION 1..122: note = Description of Artificial Sequence: Syntheticpolypeptide
EVQLVESGGGLVRPGGSLRLSCTASGFTFSTFIMFWVRQAPGKGLEYVSSISSNGGTIYYADSVKGRFTISRDNSKNTLYLQMGSLRAEDMAVYYCTRGGDFWSGYYPFDYWGQGTLVTVSS

SEQ ID NO: 31 - DNA, 24 bp, synthetic construct
  misc_feature 1..24: note = Description of Artificial Sequence: Syntheticoligonucleotide
  source 1..24: mol_type = other DNA
ggattcaccttcagtacctttatt

SEQ ID NO: 32 - protein, 8 aa, synthetic construct
  REGION 1..8: note = Description of Artificial Sequence: Syntheticpeptide
GFTFSTFI

SEQ ID NO: 33 - DNA, 24 bp, synthetic construct
  misc_feature 1..24: note = Description of Artificial Sequence: Syntheticoligonucleotide
  source 1..24: mol_type = other DNA
attagtagtaatgggggtaccata

SEQ ID NO: 34 - protein, 8 aa, synthetic construct
  REGION 1..8: note = Description of Artificial Sequence: Syntheticpeptide
ISSNGGTI

SEQ ID NO: 35 - DNA, 45 bp, synthetic construct
  misc_feature 1..45: note = Description of Artificial Sequence: Syntheticoligonucleotide
  source 1..45: mol_type = other DNA
acgagagggggcgatttttggagtggttattatccttttgactac

SEQ ID NO: 36 - protein, 15 aa, synthetic construct
  REGION 1..15: note = Description of Artificial Sequence: Syntheticpeptide
TRGGDFWSGYYPFDY

SEQ ID NO: 37 - DNA, 1347 bp, synthetic construct
  misc_feature 1..1347: note = Description of Artificial Sequence: Syntheticpolynucleotide
  source 1..1347: mol_type = other DNA
gaggtgcagctggtggagtctgggggaggcttggtccggcctggggggtccctgagactctcctgtacagcctctggattcaccttcagtacctttattatgttctgggtccgccaggctccagggaagggactggaatatgtttcatccattagtagtaatgggggtaccatatattatgcagactctgtgaagggcagattcaccatctccagagacaattccaagaacactctatatcttcaaatgggcagcctgagagctgaggacatggctgtatattattgtacgagagggggcgatttttggagtggttattatccttttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 38 - protein, 448 aa, synthetic construct
  REGION 1..448: note = Description of Artificial Sequence: Syntheticpolypeptide
EVQLVESGGGLVRPGGSLRLSCTASGFTFSTFIMFWVRQAPGKGLEYVSSISSNGGTIYYADSVKGRFTISRDNSKNTLYLQMGSLRAEDMAVYYCTRGGDFWSGYYPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - DNA, 378 bp, synthetic construct
  misc_feature 1..378: note = Description of Artificial Sequence: Syntheticpolynucleotide
  source 1..378: mol_type = other DNA
gaggtgcagctggtggagtctgggggaggcttggtaaagcctggggggtcccttagactctcctgtacagcctccggattctccttcagagacgcctggatgacctgggtccgccaggttccagggaaggggctggagtgggttggccgtattaggaacaaaattgatggtgggacgacagactacaatacacccgtgaaagacagattcaccatctcaagagatgattcaaaaaacacgctgtatctgcaaatgaacagcctgaaaaccgaggacacagccgtgtattattgtaccacagatatttggaactacgttctcttctactactacggtttggacgtctggggccaagggaccacggtcaccgtctcctca

SEQ ID NO: 40 - protein, 126 aa, synthetic construct
  REGION 1..126: note = Description of Artificial Sequence: Syntheticpolypeptide
EVQLVESGGGLVKPGGSLRLSCTASGFSFRDAWMTWVRQVPGKGLEWVGRIRNKIDGGTTDYNTPVKDRFTISRDDSKNTLYLQMNSLKTEDTAVYYCTTDIWNYVLFYYYGLDVWGQGTTVTVSS

SEQ ID NO: 41 - DNA, 24 bp, synthetic construct
  misc_feature 1..24: note = Description of Artificial Sequence: Syntheticoligonucleotide
  source 1..24: mol_type = other DNA
ggattctccttcagagacgcctgg

SEQ ID NO: 42 - protein, 8 aa, synthetic construct
  REGION 1..8: note = Description of Artificial Sequence: Syntheticpeptide
GFSFRDAW

SEQ ID NO: 43 - DNA, 30 bp, synthetic construct
  misc_feature 1..30: note = Description of Artificial Sequence: Syntheticoligonucleotide
  source 1..30: mol_type = other DNA
attaggaacaaaattgatggtgggacgaca

SEQ ID NO: 44 - protein, 10 aa, synthetic construct
  REGION 1..10: note = Description of Artificial Sequence: Syntheticpeptide
IRNKIDGGTT

SEQ ID NO: 45 - DNA, 51 bp, synthetic construct
  misc_feature 1..51: note = Description of Artificial Sequence: Syntheticoligonucleotide
  source 1..51: mol_type = other DNA
accacagatatttggaactacgttctcttctactactacggtttggacgtc

SEQ ID NO: 46 - protein, 17 aa, synthetic construct
  REGION 1..17: note = Description of Artificial Sequence: Syntheticpeptide
TTDIWNYVLFYYYGLDV

SEQ ID NO: 47 - DNA, 1359 bp, synthetic construct
  misc_feature 1..1359: note = Description of Artificial Sequence: Syntheticpolynucleotide
  source 1..1359: mol_type = other DNA
gaggtgcagctggtggagtctgggggaggcttggtaaagcctggggggtcccttagactctcctgtacagcctccggattctccttcagagacgcctggatgacctgggtccgccaggttccagggaaggggctggagtgggttggccgtattaggaacaaaattgatggtgggacgacagactacaatacacccgtgaaagacagattcaccatctcaagagatgattcaaaaaacacgctgtatctgcaaatgaacagcctgaaaaccgaggacacagccgtgtattattgtaccacagatatttggaactacgttctcttctactactacggtttggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 48 - protein, 452 aa, synthetic construct
  REGION 1..452: note = Description of Artificial Sequence: Syntheticpolypeptide
EVQLVESGGGLVKPGGSLRLSCTASGFSFRDAWMTWVRQVPGKGLEWVGRIRNKIDGGTTDYNTPVKDRFTISRDDSKNTLYLQMNSLKTEDTAVYYCTTDIWNYVLFYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 49 - DNA, 363 bp, synthetic construct
  misc_feature 1..363: note = Description of Artificial Sequence: Syntheticpolynucleotide
  source 1..363: mol_type = other DNA
caggtgcagctgcaggagtcgggcccaggactggtgaagccttcggagaccctgtccctcacctgcactgtctctgatgactccatcattagttactactggagctggatccggcagcccccagggaagggactggaatggattggatatatctattacagtgggcgcaccaactacaacccctccctcaagagtcgagtcaccatatcagtagacacgtccaagaaccaggtctcactgaagctgaactctgtgattgctgcggacacggccgtgtattactgtgcgagagtgtccgaggattcctactaccactacggtatggacgtctggggccaagggaccacggtcaccgtctcctca

SEQ ID NO: 50 - protein, 121 aa, synthetic construct
  REGION 1..121: note = Description of Artificial Sequence: Syntheticpolypeptide
QVQLQESGPGLVKPSETLSLTCTVSDDSIISYYWSWIRQPPGKGLEWIGYIYYSGRTNYNPSLKSRVTISVDTSKNQVSLKLNSVIAADTAVYYCARVSEDSYYHYGMDVWGQGTTVTVSS

SEQ ID NO: 51 - DNA, 24 bp, synthetic construct
  misc_feature 1..24: note = Description of Artificial Sequence: Syntheticoligonucleotide
  source 1..24: mol_type = other DNA
gatgactccatcattagttactac

SEQ ID NO: 52 - protein, 8 aa, synthetic construct
  REGION 1..8: note = Description of Artificial Sequence: Syntheticpeptide
DDSIISYY

SEQ ID NO: 53 - DNA, 21 bp, synthetic construct
  misc_feature 1..21: note = Description of Artificial Sequence: Syntheticoligonucleotide
  source 1..21: mol_type = other DNA
atctattacagtgggcgcacc

SEQ ID NO: 54 - protein, 7 aa, synthetic construct
  REGION 1..7: note = Description of Artificial Sequence: Syntheticpeptide
IYYSGRT

SEQ ID NO: 55 - DNA, 45 bp, synthetic construct
  misc_feature 1..45: note = Description of Artificial Sequence: Syntheticoligonucleotide
  source 1..45: mol_type = other DNA
gcgagagtgtccgaggattcctactaccactacggtatggacgtc

SEQ ID NO: 56 - protein, 15 aa, synthetic construct
  REGION 1..15: note = Description of Artificial Sequence: Syntheticpeptide
ARVSEDSYYHYGMDV

SEQ ID NO: 57 - DNA, 1344 bp, synthetic construct
  misc_feature 1..1344: note = Description of Artificial Sequence: Syntheticpolynucleotide
  source 1..1344: mol_type = other DNA
caggtgcagctgcaggagtcgggcccaggactggtgaagccttcggagaccctgtccctcacctgcactgtctctgatgactccatcattagttactactggagctggatccggcagcccccagggaagggactggaatggattggatatatctattacagtgggcgcaccaactacaacccctccctcaagagtcgagtcaccatatcagtagacacgtccaagaaccaggtctcactgaagctgaactctgtgattgctgcggacacggccgtgtattactgtgcgagagtgtccgaggattcctactaccactacggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 58 - protein, 447 aa, synthetic construct
  REGION 1..447: note = Description of Artificial Sequence: Syntheticpolypeptide
QVQLQESGPGLVKPSETLSLTCTVSDDSIISYYWSWIRQPPGKGLEWIGYIYYSGRTNYNPSLKSRVTISVDTSKNQVSLKLNSVIAADTAVYYCARVSEDSYYHYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 124 aa, synthetic construct
  REGION 1..124: note = Description of Artificial Sequence: Syntheticpolypeptide
QVQLVESGGGVVQPGRSLRLSCAASGFTFSSYGMHWVRQAPGKGLEWVAVISYAGNNKYYADSVKGRFTVSRDNSKKTLYLQMNSLRSEDTAVYYCAKDSYYDFLTDPDVLDIWGQGTMVTVSS

SEQ ID NO: 60 - protein, 8 aa, synthetic construct
  REGION 1..8: note = Description of Artificial Sequence: Syntheticpeptide
GFTFSSYG

SEQ ID NO: 61 - protein, 8 aa, synthetic construct
  REGION 1..8: note = Description of Artificial Sequence: Syntheticpeptide
ISYAGNNK

SEQ ID NO: 62 - protein, 17 aa, synthetic construct
  REGION 1..17: note = Description of Artificial Sequence: Syntheticpeptide
AKDSYYDFLTDPDVLDI

SEQ ID NO: 63 - protein, 118 aa, synthetic construct
  REGION 1..118: note = Description of Artificial Sequence: Syntheticpolypeptide
EVQLVESGGGLVQPGGSLRLSCAASGFTFSRNNMHWVRQAPGKGLEYVSGISSNGGRTYYADSVKGRFTISRDNSKNTLYLQMGGLRAADMAVYFCTRDDELLSFDYWGQGTLVTVSS

SEQ ID NO: 64 - protein, 8 aa, synthetic construct
  REGION 1..8: note = Description of Artificial Sequence: Syntheticpeptide
GFTFSRNN

SEQ ID NO: 65 - protein, 8 aa, synthetic construct
  REGION 1..8: note = Description of Artificial Sequence: Syntheticpeptide
ISSNGGRT

SEQ ID NO: 66 - protein, 11 aa, synthetic construct
  REGION 1..11: note = Description of Artificial Sequence: Syntheticpeptide
TRDDELLSFDY

SEQ ID NO: 67 - protein, 108 aa, synthetic construct
  REGION 1..108: note = Description of Artificial Sequence: Syntheticpolypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 68 - protein, 6 aa, synthetic construct
  REGION 1..6: note = Description of Artificial Sequence: Synthetic6xHis tag
HHHHHH

SEQ ID NO: 69 - protein, 660 aa, synthetic construct
  REGION 1..660: note = Description of Artificial Sequence: Syntheticpolypeptide
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIACPGGEQKLISEEDLGGEQKLISEEDLSGHHHHHHSSG

SEQ ID NO: 70 - protein, 24 aa, synthetic construct
  REGION 1..24: note = Description of Artificial Sequence: Syntheticpolypeptide
LHILPVAFRGDSFTHTPPLDPQEL

SEQ ID NO: 71 - protein, 18 aa, synthetic construct
  REGION 1..18: note = Description of Artificial Sequence: Syntheticpolypeptide
LEIIRGRTKQHGQFSLAV

SEQ ID NO: 72 - protein, 22 aa, synthetic construct
  REGION 1..22: note = Description of Artificial Sequence: Syntheticpolypeptide
CNVESIQWRDIVSSDFLSNMSM

SEQ ID NO: 73 - protein, 117 aa, synthetic construct [sequence omitted]

SEQ ID NO: 74 - protein, 107 aa, synthetic construct [sequence omitted]

SEQ ID NO: 75 - protein, 8 aa, synthetic construct
  REGION 1..8: note = Synthetic: HCDR1
GFTFSNFG

SEQ ID NO: 76 - protein, 8 aa, synthetic construct
  REGION 1..8: note = Synthetic: HCDR2
ISGGGRDT

SEQ ID NO: 77 - protein, 10 aa, synthetic construct
  REGION 1..10: note = Synthetic: HCDR3
VKWGNIYFDY

SEQ ID NO: 78 - protein, 6 aa, synthetic construct
  REGION 1..6: note = Synthetic: LCDR1
LSINTF

SEQ ID NO: 79 - skipped

SEQ ID NO: 80 - protein, 9 aa, synthetic construct
  REGION 1..9: note = Synthetic: LCDR3
QQSSNTPFT

SEQ ID NO: 81 - protein, 444 aa, synthetic construct
  REGION 1..444: note = Synthetic: aa1-117: HCVR, aa118-444: HC constant
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 82 - protein, 214 aa, synthetic construct
  REGION 1..214: note = Synthetic: aa1-107: LCVR, aa108-214: LC constant
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0270849 A1
(43) Pub. Date: Aug. 15, 2024

(54) METHODS OF TREATING CANCER WITH BISPECIFIC EGFR XCD28 ANTIBODIES ALONE OR IN COMBINATION WITH ANTI-PD-1 ANTIBODIES

(71) Applicant: Regeneron Pharmaceuticals, Inc., Tarrytown, NY (US)

(72) Inventors: Melissa D. Mathias, Brooklyn, NY (US); Matthew G. Fury, New York, NY (US); Israel Lowy, Dobbs Ferry, NY (US); Frank Seebach, Montclair, NJ (US)

(21) Appl. No.: 18/479,223

(22) Filed: Oct. 2, 2023

Related U.S. Application Data
(60) Provisional application No. 63/495,189, filed on Apr. 10, 2023.
(60) Provisional application No. 63/380,991, filed on Oct. 26, 2022.
(60) Provisional application No. 63/378,102, filed on Oct. 3, 2022.

(51) Int. Cl. C07K 16/28; A61K 39/00; A61P 35/00

(52) U.S. Cl. CPC C07K 16/2818; A61P 35/00; C07K 16/2863; A61K 2039/505; C07K 2317/31

(57) ABSTRACT

The present disclosure provides methods of treating cancer, using multispecific antibodies or antigen-binding fragments thereof that bind to EGFR and CD28 (EGFRxCD28). Such antibodies may be combined with a further therapeutic agent such as an anti-PD-1 antibody, e.g., cemiplimab. Methods for treating cancers (e.g., EGFR-expressing cancer) by administering the antibodies (e.g., and combinations thereof with anti-PD-1) are also provided.

### RELATED APPLICATIONS

This application is related to and claims priority of U.S. Provisional Application No. 63/378,102, filed on Oct. 3, 2022, U.S. Provisional Application No. 63/380,991, filed on Oct. 26, 2022 and U.S. Provisional Application No. 63/495,189, filed on Apr. 10, 2023. The entire contents of each of the foregoing applications are expressly incorporated herein by reference.

### FIELD

The present disclosure relates to methods of treating or preventing cancer (e.g., EGFR-expressing cancer) in a subject (e.g., a human) in need thereof, comprising administering to the subject an effective amount of a bispecific antibody that binds to epidermal growth factor (EGF) receptor and CD28, or an antigen-binding fragment thereof, in combination with an anti-PD-1 antibody or antigen-binding fragment thereof.

### SEQUENCE LISTING

The instant application contains a Sequence Listing which has been submitted electronically in XML format and is hereby incorporated by reference in its entirety. Said XML copy, created on Oct. 1, 2023, is named 118003-91004.XML and is 117,441 bytes in size.

### BACKGROUND

The ability of T-cells to recognize and kill their cellular targets—such as virally—infected cells or tumor cells—depends on a coordinated set of interactions. Foremost among these is the recognition and binding of the target cell by the T-cell Receptor (TCR) complex (which includes the associated CD3 γ, δ, ϵ and ζ chains), and this interaction has been referred to as “signal 1” for T-cell activation. The TCR recognizes a viral or tumor peptide presented on the groove of an MHC protein expressed on the surface of the target cell. Because such binding is generally of low-affinity, successful triggering of “signal 1” requires clustering of many TCR complexes along the interface between the T-cell and its target cell; this interface has been referred to as the “immune synapse”. T-cell activation can be further promoted by additional interactions. For example, T-cells have a molecule referred to as CD28 on their surface, which can provide a co-stimulatory “signal 2” to augment the activation via the TCR complex. When a T-cell recognizes its target cell via its TCR complex, and then also engages “signal 2” via CD28 binding to its cognate ligand(s) on the target cell, T-cell activation is enhanced; as with “signal 1”, CD28-mediated “signal 2” is thought to occur via co-clustering at the immune synapse.

Agonistic anti-CD28 monoclonal antibodies can be applied in sustained ex vivo expansion of cultured T-cells; however, the use of antibodies against CD28 has been discouraged as a result of a series of acute and serious adverse events in a phase I clinical trial where super agonist anti-CD28 monoclonal antibodies was tested systemically (Hünig, Nature Reviews Immunology. 2012;12:317-318). Localized or targeted use of anti-CD28 monoclonal antibody can be used with less risk for promotion of antitumor immunity. Jung et al., Int J Cancer. 2001 Jan 15;91(2):225-30.

Different families of growth factors and growth factor receptors have been shown to be involved in the autonomous growth of cancer cells. Among these, the epidermal growth factor receptor (EGFR) and the EGF-family of peptide growth factor have a central role in the pathogenesis and progression of different carcinoma types. EGFR belongs to a family of receptors that encompasses three additional proteins, ErbB-2, ErbB-3 and ErbB-4. These proteins and the growth factors of the EGF family form an integrated system in which a signal that hits an individual receptor type is often transmitted to other receptors of the same family.

Monoclonal antibodies (mAbs) aimed at enhancing T-cell activation are under clinical development as anti-tumor therapeutics. The majority of current treatments, however, have a difficult time overcoming the inhibitory nature of the tumor microenvironment, thus failing to generate efficient tumor-specific T-cell activation and subsequent tumor cell killing. Several blocking mAbs directed against checkpoint inhibitors such as CTLA-4 (cytotoxic T lymphocyte-associated protein) and programmed cell death 1 (PD-1)/programmed cell death ligand 1 (PD-L1) have been clinically approved for melanoma, renal cell carcinoma, non-small lung cancer and advanced metastatic cutaneous squamous cell carcinoma. Blocking PD-1 releases the break on T-cell activation, but its efficacy as a single agent often it is not sufficient to get tumor clearance and durable anti-tumor responses.

### SUMMARY

The present disclosure provides a method for treating a cancer in a subject, the method comprising administering to the subject a therapeutically effective amount of a bispecific antibody or antigen-binding fragment thereof comprising a first antigen-binding domain that binds cluster of differentiation factor 28 (CD28) and a second antigen-binding domain that binds epidermal growth factor receptor (EGFR) in combination with an antibody or antigen-binding fragment thereof that specifically binds programmed death receptor-1 (PD-1), thereby treating the cancer in the subject.

In some embodiments, the cancer is a solid tumor. In certain embodiments, the cancer is a EGFR-expressing cancer. In some embodiments, the cancer is selected from esophageal carcinoma, lung squamous cell carcinoma, lung adenocarcinoma, cervical cancer (including cervical squamous cell carcinoma), endometrial adenocarcinoma, bladder cancer, urothelial carcinoma, lung cancer, non-small cell lung cancer (NSCLC), colorectal cancer (such as microsatellite-stable colorectal cancer), sigmoid colon adenocarcinoma, rectal cancer, endometrial cancer, skin cancer, head & neck squamous cell carcinoma, brain cancer, glioblastoma multiforme, non-CNS tumor, cutaneous squamous cell carcinoma, breast cancer, gastric cancer, gastroesophageal cancer, gastroesophageal adenocarcinoma, pancreatic cancer, prostate cancer, ovarian cancer, melanoma, nasopharyngeal carcinoma, anal carcinoma, mesothelioma, renal cell carcinoma, gallbladder/cholangiocarcinoma, pancreatic carcinoma, penile squamous cell carcinoma, or vulvovaginal carcinoma. In one embodiment, the cancer is cervical cancer. In one embodiment, the cervical cancer is cervical squamous cell carcinoma. In one embodiment, the cancer is microsatellite-stable colorectal cancer with liver metastases. In one embodiment, the cancer is microsatellite-stable colorectal cancer without liver metastases. In one embodiment, the cancer is microsatellite stable colorectal cancer (MSS-CRC) with active liver and/or peritoneal metastases. In one embodiment, the cancer is MSS-CRC with lung/lymph node metastases. In one embodiment, the cancer is EGFR-mutant NSCLC post-third generation tyrosine kinase inhibitor (TKI). In one embodiment, the cancer is EGFR-mutant NSCLC post-third generation TKI and post-platinum-doublet chemotherapy. In one embodiment, the cancer is cutaneous squamous cell carcinoma. In one embodiment, the cancer is triple-negative breast cancer.

In some embodiments, the method further comprises selecting a subject, wherein the subject has an advanced solid tumor. In some embodiments, the subject has at least one of the following criteria, or is selected on the basis of at least one of the following criteria: (1) has metastatic disease, or locally advanced disease that is not a candidate for curative surgery or curative radiation; (2) is not a candidate for an approved indication of an anti-PD-1 or PD-L1 therapy, or such therapy is otherwise not available to the subject (alone or in combination); (3) has exhausted all therapeutic options that are expected to provide meaningful clinical benefit, either through disease relapse, treatment refractory disease, or intolerance except subjects with malignancies where anti-PD-1/PD-L1 therapies have demonstrated clinical benefit; and/or (4) has any of the following cancer types: (a) colorectal cancer that is microsatellite stable as documented by local pathology; (b) gastric or gastroesophageal junction cancer; (c) esophageal cancer; (d) breast cancer (ductal or lobular carcinoma, regardless of receptor status); (e) non-small-cell lung cancer (NSCLC) (any PD-L1 expression); (f) head and neck squamous cell carcinoma (SCC); (g) nasopharyngeal carcinoma; (h) cervical carcinoma; (i) anal carcinoma; (j) mesothelioma; (k) prostate adenocarcinoma; (I) renal cell carcinoma (chromophobe, clear cell, or papillary); (m) gallbladder/cholangiocarcinoma; (n) urothelial carcinoma; (o) pancreatic carcinoma; (p) penile SCC; (q) vulvovaginal carcinoma; or (r) additional non-CNS tumor types for which elevated EGFR expression in the tumor is demonstrated.

In some embodiments, the subject has been treated with a prior therapy selected from radiation, surgery, chemotherapy, a PD-1 inhibitor, a PD-L1 inhibitor, an anti-VEGF therapy, CAR-T therapy, and/or an anti-EGFR therapy. In some embodiments, the subject has not received prior anti-PD-1 therapy or anti-PD-L1 therapy.

In some embodiments, the subject has microsatellite-stable colorectal cancer (MSS CRC). In some embodiments, the subject with microsatellite-stable colorectal cancer has, or is selected on the basis of, at least one of the following attributes: (a) has metastatic CRC; (b) is not a candidate for curative surgery or curative radiation; (c) may have active metastases are present in the liver and/or peritoneum at the time of screening; (d) no active metastases have been identified in the liver or peritoneum at the time of screening and the only sites of disease are present in lung(s) and/lung or lymph nodes; (e) has microsatellite stable as documented by pathology report; (f) has received at least one line of therapy in the relapsed/metastatic setting, wherein the therapy comprises anti-EGFR therapy or anti-VEGF therapy; or (g) is anti-PD-1/PD-L1 naïve, defined as never having previously been treated with a drug that targets PD-1.

In some embodiments, the subject has triple negative breast cancer (TNBC). In some embodiments, the subject with TNBC has, or is selected on the basis of, at least one of the following attributes: (a) has metastatic TNBC; (b) is not a candidate for curative surgery or curative radiation; (c) is not a candidate for anti-PD-1 or anti-PD-L1 therapy in an approved indication, or such therapy is otherwise not available to the subject; (d) has triple negative cancer (ER-/PR-/Her2-), as documented by pathology report; or (e) is anti-PD-1/PD-L1 naïve, defined as never having previously been treated with a drug that targets PD-1.

In some embodiments, the subject has cutaneous squamous cell carcinoma (CSCC), wherein (i) the subject is not a candidate for curative surgery or curative radiation or (ii) the subject is anti-PD-1/PD-L1 naïve, defined as never having previously been treated with a drug that targets PD-1.

In some embodiments, the subject has non-small cell lung cancer (NSCLC). In some embodiments, the subject has, or is selected on the basis of, at least one of the following attributes:(a) the subject has previously documented histologically or cytologically documented locally advanced or metastatic EGFR mutated non-squamous NSCLC disease; (b) has advanced or metastatic NSCLC; (c) is not a candidate for curative surgery or curative radiation; (d) has a previously documented targetable EGFR mutation (EGFR Exon 19 deletion, EGFR L858R mutation, EGFR exon20 insertion, or exon 18/21 atypical mutations); (e) is chemotherapy naïve; (f) has received treatment with platinum-doublet chemotherapy; (e) has received treatment with a third generation TKI; or (g) is anti-PD-1/PD-L1 naïve, defined as never having previously been treated with a drug that targets PD-1.

In some embodiments, the subject has head and neck squamous cell carcinoma (HNSCC). In some embodiments, the subject has, or is selected on the basis of, at least one of the following attributes: (a) has advanced or metastatic disease; (b) is not a candidate for curative surgery or curative radiation; (c) has PD-L1 expression of CPS ≥1% by a local IHC assay; (d) has received no prior systemic treatment for recurrent or metastatic HNSCC; and/or (e) is anti-PD-1/PD-L1 naïve, defined as never having previously been treated with a drug that targets PD-1.

In certain embodiments, the bispecific EGFRxCD28 antibody or antigen-binding fragment thereof is administered at a dose of about 0.1 mg to about 3000 mg. In some embodiments, the bispecific EGFRxCD28 antibody or antigen-binding fragment thereof is administered at a dose of about 0.01 mg, 0.03 mg, 0.05 mg, 0.1 mg, 0.3 mg 0.5 mg, 1 mg, 2 mg, 3 mg, 4 mg, 5 mg, 6 mg, 8 mg, 10 mg, 15 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1000 mg, 1200 mg, 1500 mg, 1800 mg, 2000 mg, 2400 mg, 2700 mg, or 3000 mg.

In certain embodiments, the anti-PD-1 antibody or antigen-binding fragment thereof is administered at a dose of about 50 mg to about 1500 mg. In some embodiments, the anti-PD-1 antibody is administered at a dose of 350 mg.

In some embodiments, the method comprises administering one or more doses of the bispecific EGFRxCD28 antibody or antigen-binding fragment thereof in combination with one or more doses of the anti-PD-1 antibody or antigen-binding fragment thereof.

In some embodiments, each of the one or more doses of the bispecific EGFRxCD28 antibody or antigen-binding fragment thereof is about 0.1 mg to about 3000 mg. In some embodiments, each of the one or more doses is about 0.01 mg, 0.03 mg, 0.05 mg, 0.1 mg, 0.3 mg, 0.5 mg, 1 mg, 2 mg, 3 mg, 4 mg, 5 mg, 6 mg, 8 mg, 10 mg, 15 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1000 mg, 1200 mg, 1500 mg, 1800 mg, 2000 mg, 2400 mg, 2700 mg, or 3000 mg.

In some embodiments, each of the one or more doses of the anti-PD-1 antibody or antigen-binding fragment thereof is about 50 mg to about 1500 mg. In some embodiments, each of the one or more doses of the anti-PD-1 antibody is 350 mg.

In certain embodiments, each of the one or more doses of the bispecific EGFRxCD28 antibody or antigen-binding fragment thereof and/or the one or more doses of the anti-PD-1 antibody or antigen-binding fragment thereof is administered 0.5 to 14 weeks after the immediately preceding dose.

In some embodiments, each of the one or more doses of the bispecific EGFRxCD28 antibody or antigen-binding fragment thereof and/or the one or more doses of the anti-PD-1 antibody or antigen-binding fragment thereof is administered once every week, once every two weeks, once every three weeks, once every four weeks, once every five weeks or once every six weeks.

In some embodiments, each dose of the bispecific EGFRxCD28 antibody or antigen-binding fragment thereof is administered once every week. In some embodiments, each of the one or more doses of the bispecific EGFRxCD28 antibody or antigen-binding fragment thereof is administered once every three weeks.

In some embodiments, each of the one or more doses of the anti-PD-1 antibody or antigen-binding fragment thereof is administered once every three weeks.

In certain embodiments, the bispecific EGFRxCD28 antibody or antigen-binding fragment thereof and/or the anti-PD-1 antibody or antigen-binding fragment thereof are administered intravenously. In some embodiments, the bispecific EGFRxCD28 antibody or antigen-binding fragment thereof and/or the anti-PD-1 antibody or antigen-binding fragment thereof are administered subcutaneously.

In certain embodiments, the bispecific EGFRxCD28 antibody or antigen-binding fragment thereof and the anti-PD-1 antibody or antigen-binding fragment thereof are administered on the same day. In some embodiments, the bispecific EGFRxCD28 antibody or antigen-binding fragment thereof and the anti-PD-1 antibody or antigen-binding fragment thereof are administered on different days.

In certain embodiments, the bispecific EGFRxCD28 antibody or antigen-binding fragment thereof is administered before or after the anti-PD-1 antibody or antigen-binding fragment thereof.

In certain aspects, the method of the disclosure comprises the steps of: (i) administering to the subject the bispecific EGFRxCD28 antibody or antigen-binding fragment thereof at a dose of 0.1 mg to 3000 mg subcutaneously or intravenously once every week or once every 3 weeks for a period of monotherapy, wherein the period of monotherapy is at least 3 weeks; and (ii) administering to the subject the bispecific EGFRxCD28 antibody or antigen-binding fragment thereof at a dose of 0.1 mg to 3000 mg subcutaneously or intravenously once every week or once every 3 weeks, and administering to the subject an anti-PD-1 antibody or antigen-binding fragment thereof at a dose of 150 mg to 500 mg intravenously or subcutaneously once every 3 weeks.

In some embodiments, the period of monotherapy is at least 3 weeks, at least 4 weeks, at least 5 weeks, or at least 6 weeks. In some embodiments, the period of monotherapy is less than a year, less than 9 months, less than 6 months, less than 3 months, less than 6 weeks, or less than 1 month.

In some embodiments, during step (ii), the anti-PD-1 antibody or antigen-binding fragment thereof is administered on a different day as the bispecific EGFRxCD28 antibody or antigen-binding fragment thereof. In some embodiments, during step (ii), the anti-PD-1 antibody or antigen-binding fragment thereof is administered on the same day as the bispecific EGFRxCD28 antibody or antigen-binding fragment thereof.

In certain embodiments, the method further comprises administering to the subject one or more additional agents to treat one or more symptoms of an immune-related adverse event. In some embodiments, the one or more additional agents comprise an IL-6 receptor inhibitor (e.g., an anti-IL6R antibody), a corticosteroid, and/or a non-steroidal anti-inflammatory drug (NSAID).

In some embodiments, the subject has stable disease, partial response, or complete response upon administration of the bispecific antibody or antigen-binding fragment thereof for at least one week at a dose of about 0.1 mg to about 3000 mg in combination with the anti-PD-1 antibody or antigen-binding fragment thereof.

In one embodiment, the subject has cervical cancer and has a partial response after administration. In one embodiment, the subject is a PD-1-negative patient, wherein the subject has cervical cancer and achieves a partial response after administration.

In one embodiment, the subject has microsatellite-stable colorectal cancer (CRC) and has stable disease or a partial response after administration. In one embodiment, the subject is administered the bispecific antibody, or antigen-binding fragment thereof, in combination with cemiplimab, wherein the subject has CRC and achieves stable disease or a partial response after administration.

In some embodiments, the anti-PD-1 antibody or antigen-binding fragment thereof is cemiplimab, nivolumab, pembrolizumab, MEDI0608, BI 754091, spartalizumab (PDR001), camrelizumab (SHR-1210), JNJ-63723283, MCLA-134, toripalimab, sintilimab, tislelizumab, serplulimab, dostarlimab, retifanlimab, zimberelimab, penpulimab, pidilizumab, HX008, balstilimab or ezabenlimab, or an antigen-binding fragment of any of the foregoing.

In certain embodiments, the anti-PD-1 antibody or antigen-binding fragment thereof comprises the heavy chain complementarity determining regions (HCDR1, HCDR2 and HCDR3) of a heavy chain variable region (HCVR) comprising the amino acid sequence of SEQ ID NO: 73 and three light chain complementarity determining regions (LCDR1, LCDR2 and LCDR3) of a light chain variable region (LCVR) comprising the amino acid sequence of SEQ ID NO: 74.

In some embodiments, the anti-PD-1 antibody or antigen-binding fragment thereof comprises three HCDRs (HCDR1, HCDR2 and HCDR3) and three LCDRs (LCDR1, LCDR2 and LCDR3), wherein HCDR1 comprises the amino acid sequence of SEQ ID NO: 75; HCDR2 comprises the amino acid sequence of SEQ ID NO: 76; HCDR3 comprises the amino acid sequence of SEQ ID NO: 77; LCDR1 comprises the amino acid sequence of SEQ ID NO: 78; LCDR2 comprises the amino acid sequence of SEQ ID NO: 79 (Ala Ala Ser, or AAS); and LCDR3 comprises the amino acid sequence of SEQ ID NO: 80.

In some embodiments, the HCVR comprises the amino acid sequence of SEQ ID NO: 73 and the LCVR comprises the amino acid sequence of SEQ ID NO: 74.

In some embodiments, the anti-PD-1 antibody comprises a heavy chain comprising the amino acid sequence of SEQ ID NO: 81 and a light chain comprising the amino acid sequence of SEQ ID NO: 82.

In some embodiments, the anti-PD-1 antibody is cemiplimab, or an antigen-binding fragment thereof.

In some embodiments, the methods comprise administration of any one of the bispecific EGFRxCD28 antibodies disclosed herein

In certain embodiments, the first antigen-binding domain that binds CD28 comprises three heavy chain complementarity determining regions (CDR-H1, CDR-H2 and CDR-H3) contained within a heavy chain variable region (HCVR) comprising the amino acid sequence of SEQ ID NO: 10; and three light chain complementarity determining regions (CDR-L1, CDR-L2 and CDR-L3) contained within a light chain variable region (LCVR) comprising the amino acid sequence of SEQ ID NO: 16.

In some embodiments, the first antigen-binding domain's CDR-H1 comprises the amino acid sequence: GGSISSYY (SEQ ID NO: 12), CDR-H2 comprises the amino acid sequence: IYYSGIT (SEQ ID NO: 6), and CDR-H3 comprises the amino acid sequence: ARWGVRRDYYYYGMDV (SEQ ID NO: 14).

In some embodiments, the first antigen-binding domain's CDR-L1 comprises the amino acid sequence: QSVSSSY (SEQ ID NO: 18), CDR-L2 comprises the amino acid sequence: GAS (SEQ ID NO: 20), and CDR-L3 comprises the amino acid sequence: QQYGSSPWT (SEQ ID NO: 22).

In some embodiments, the first antigen-binding domain comprises a HCVR comprising the amino acid sequence of SEQ ID NO: 10, and a LCVR comprising the amino acid sequence of SEQ ID NO: 16.

In certain embodiments, the second antigen binding domain that binds human EGFR comprises three heavy chain complementarity determining regions (CDR-H1, CDR-H2 and CDR-H3) contained within a heavy chain variable region (HCVR) comprising the amino acid sequence of SEQ ID NO: 2; and three light chain complementarity determining regions (CDR-L1, CDR-L2 and CDR-L3) contained within a light chain variable region (LCVR) comprising the amino acid sequence of SEQ ID NO: 16.

In some embodiments, the second antigen binding domain's CDR-H1 comprises the amino acid sequence: GDSIITFY (SEQ ID NO: 4), CDR-H2 comprises the amino acid sequence: IYYSGIT (SEQ ID NO: 6), and CDR-H3 comprises the amino acid sequence: ARVSEDSYFHYGMDV (SEQ ID NO: 8).

In some embodiments, the second antigen binding domain's CDR-L1 comprises the amino acid sequence: QSVSSSY (SEQ ID NO: 18); CDR-L2 comprises the amino acid sequence: GAS (SEQ ID NO: 20); and CDR-L3 comprises the amino acid sequence: QQYGSSPWT (SEQ ID NO: 22).

In some embodiments, the second antigen-binding domain comprises a HCVR comprising the amino acid sequence of SEQ ID NO: 2, and a LCVR comprising the amino acid sequence of SEQ ID NO: 16.

In certain aspects, the first antigen binding domain that binds human CD28 comprises a heavy chain variable region that comprises the amino acid sequence set forth in SEQ ID NO: 10, and a light chain variable region that comprises the amino acid sequence set forth in SEQ ID NO: 16; and the second antigen binding domain that binds human EGFR comprises a heavy chain variable region that comprises the amino acid sequence set forth in SEQ ID NO: 2; and a light chain variable region that comprises the amino acid sequence set forth in SEQ ID NO: 16.

In some embodiments, the bispecific antibody comprises a first heavy chain comprising the amino acid sequence of SEQ ID NO: 26.

In some embodiments, the bispecific antibody comprises a second heavy chain comprising the amino acid sequence of SEQ ID NO: 24.

In some embodiments, the bispecific antibody comprises a light chain comprising the amino acid sequence of SEQ ID NO: 28.

In some embodiments, the bispecific antibody comprises a first heavy chain comprising the amino acid sequence of SEQ ID NO: 26, a second heavy chain comprising the amino acid sequence of SEQ ID NO: 24, and a common light chain comprising the amino acid sequence of SEQ ID NO: 28.

In some embodiments, the first antigen-binding domain comprises a heavy chain comprising the amino acid sequence of SEQ ID NO: 26 and a light chain comprising the amino acid sequence of SEQ ID NO: 28.

In some embodiments, the second antigen-binding domain comprises a heavy chain comprising the amino acid sequence of SEQ ID NO: 24 and a light chain comprising the amino acid sequence of SEQ ID NO: 28.

In some embodiments, the bispecific EGFRxCD28 antibody is REGN7075, or an antigen-binding fragment thereof.

In some embodiments, the method further comprises administering chemotherapy to the subject. In one embodiment, the chemotherapy is platinum-based chemotherapy.

### BRIEF DESCRIPTION OF THE DRAWINGS

FIG. 1 shows a schematic representation of a proposed mechanism of REGN7075 and cemiplimab combination therapy. REGN7075 (EGFRxCD28) binds EGFR on tumor cells and CD28 on T-cells thereby bridging the two cell types. Cemiplimab (anti-PD-1) binds to PD-1 on T-cells to prevent PD-1 mediated suppressive signals. Tumor associated antigens on MHC are presented to T-cells.

FIGS. 2A-2D show study flow diagram for REGN7075 weekly dosing with REGN7075 monotherapy lead-in period (FIG. 2A); study flow diagram for cohorts REGN7075 weekly dosing, concurrent start with cemiplimab (FIG. 2B); study flow diagram for dose escalation cohorts with a REGN7075 monotherapy lead-in period and subsequent Q3W dosing (FIG. 2C); and study flow diagram for dose escalation (FIG. 2D). REGN7075 may be administered as a split dose or step-up dosing schedule, as disclosed elsewhere herein.

FIG. 3 shows a schematic of a dose escalation study to evaluate the safety, tolerability, pharmacokinetics, and preliminary antitumor activity of REGN7075 (EGFRxCD28) in combination with cemiplimab (anti-PD-1) in subjects with advanced solid tumors. DL: dose level; RP2D: recommended Phase II dose.

FIG. 4 shows mean serum concentration of REGN7075 following IV administration of the first dose of REGN7075. DL1, 0.03 mg; DL2, 0.1 mg; DL3, 0.3 mg; DL4, 1 mg; DL5, 3 mg; DL6, 10 mg; DL7, 30 mg.

FIGS. 5A-5B show T-cell activation-associated cytokines in serum of patients with detectable IL-2 (FIG. 5A) and IFN-γ (FIG. 5B) who received REGN7075 alone and in combination with cemiplimab. DL, dose level; EOT, end of treatment; ET, early termination; IFN, interferon; IL, interleukin; IV, intravenous; LLOQ, lower limit of quantitation; Q3W, every 3 weeks.

FIG. 6 shows percent change in target lesions from baseline in 18 patients treated in dose escalation. The data is preliminary and trial is still on going. DL, dose level; MSS CRC, microsatellite-stable colorectal cancer.

FIG. 7 shows percent change from base line in target lesions in 25 patients treated in dose eslacation based on data as of December, 2022. The data is preliminary and trial is still on going. SD: stable disease; PD: progressive disease; CR/PR: complete response/partial response.

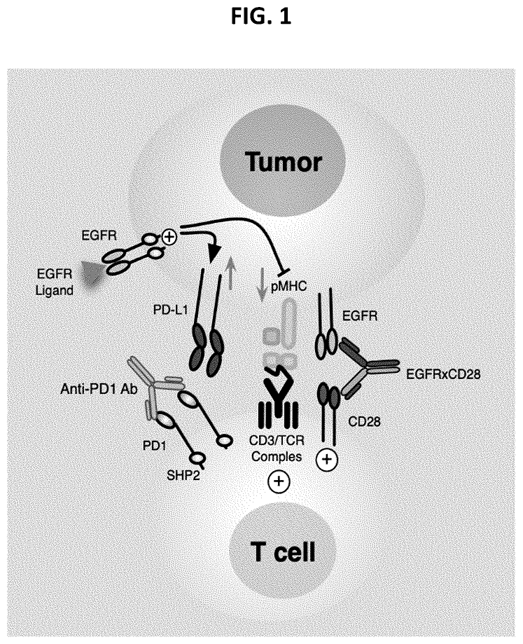

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 1 01 and all values in between (e.g., 99.1, 99.2, 99.3, 99.4, etc.).

Although any methods and materials similar or equivalent to those described herein can be used in the practice or testing of the present disclosure, the preferred methods and materials are now described. All patents, applications and non-patent publications mentioned in this specification are incorporated herein by reference in their entireties.

### Definitions

“EGFR” and “EGFR fragment,” as used herein refer to the well-known human EGFR protein or a fragment thereof unless specified as being from a non-human species (e.g., “mouse EGFR,” “mouse EGFR fragment,” “monkey EGFR,” “monkey EGFR fragment,” etc.). In one embodiment, human EGFR comprises the amino acid sequence set forth in NCBI accession no. NP_005219.2. In one embodiment, human EGFR (amino acids L25-A647 of Accession number 005228.4) is shown with a C-terminal CPGG.myc epitope (E1-L10).GlyGly.myc epitope (E1-L10).SerGly.6XHis.SSG tag (SEQ ID NO: 69).

“CD28,” as used herein, refers to the well-known human CD28 protein which is express on T cells as a costimulatory receptor unless specified as being from a non-human species. In one embodiment, human CD28 comprises the amino acid sequence as set forth NCBI accession No. NP_006130.1.

“PD-1” or “programmed death receptor-1,” as used herein, refers to the well-known human PD-1 protein, or fragment thereof, unless specified as being from a non-human species. In one embodiment, the human PD-1 comprises the amino acid sequence as set forth in NP_005009.

An “antibody” is an immunoglobulin molecule comprising four polypeptide chains, two heavy chains (HC) and two light chains (LC) inter-connected by disulfide bonds. Each heavy chain (HC) comprises a heavy chain variable region (abbreviated herein as HCVR or V_{H}) and a heavy chain constant region (e.g., IgG, IgG1 or IgG4). The heavy chain constant region comprises three domains, C_{H}1, C_{H}2 and C_{H}3. Each light chain (LC) comprises a light chain variable region (abbreviated herein as LCVR or V_{L}) and a light chain constant region (e.g., lambda or kappa). The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} includes three CDRs and four FRs, arranged from amino-terminus to carboxy-terminus in the following order: FR1, CDR1, FR2, CDR2, FR3, CDR3, FR4. A heavy chain CDR may be referred to as HCDR and a light chain CDR may be referred to as LCDR. In different embodiments of the disclosure, the FRs of an antibody (or antigen-binding portion thereof) may be identical to the human germline sequences, or may be naturally or artificially modified.

An antibody which, for example, is bispecific includes an arm that binds to a first antigen and another arm that binds to a second antigen. For example, an EGFRxCD28 bispecific antibody includes one arm that binds EGFR and another art that binds to CD28. Bispecific antigen-binding molecules (e.g., bispecific antibodies or antigen-binding fragments or portions thereof) may have an effector arm that binds to a first antigen and a targeting arm that binds to second antigen. The effector arm may be the first antigen-binding domain (e.g., anti-CD28) that binds to the antigens on effector cells (e.g., T cells). The targeting arm may be the second antigen binding domain (e.g., anti-EGFR antibody) that binds to the antigens on target cells (e.g., tumor cells). According to certain exemplary embodiments, the effector arm binds to CD28 and the targeting arm binds to EGFR. The terms “EGFRxCD28 bispecific antibody”, “bispecific EGFRxCD28 antibody” and EGFRxCD28 are used interchangeably throughout this specification.

An antigen-binding arm of a Y-shaped IgG antibody (e.g., a CD28 or EGFR binding arm) refers to a structural portion of the antibody that confers binding specificity to the antigen. For example, an antigen-binding arm of an IgG antibody has a heavy chain (HC) associated with a light chain (LC).

An “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. A multispecific antigen-binding fragment of an antibody binds to multiple antigens (e.g., two different antigens if the fragment is bispecific). Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Non-limiting examples of antigen-binding fragments include: (i) Fab fragments; (ii) F(ab′)₂ fragments; (iii) Fd fragments; (iv) Fv fragments; (v) single-chain Fv (scFv) molecules; and (vi) dAb fragments.

An antigen-binding fragment of an antibody will, in one embodiment, comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antigen-binding proteins (e.g., antibodies or antigen-binding fragments).

The term “recombinant” antigen-binding proteins, such as antibodies or antigen-binding fragments thereof, refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a host cell (e.g., Chinese hamster ovary (CHO) cell) or cellular expression system or isolated from a recombinant combinatorial human antibody library. The present disclosure includes recombinant antigen-binding proteins as set forth herein.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. In some embodiments, the term “specifically binds” or “binds specifically” refers to those antigen-binding proteins (e.g., antibodies or antigen-binding fragments thereof) having a binding affinity to an antigen, such as EGFR, CD28, or PD-1 protein, expressed as K_{D}, of less than about 10⁻⁶ M (e.g., 10⁻⁷ M, 10⁻⁸ M, 10⁻⁹ M, 10⁻¹⁰ M, 10⁻¹¹ M or 10⁻¹² M), as measured by, e.g., real-time, label free bio-layer interferometry assay, for example, at 25° C. or 37ºC, e.g., an Octet® HTX biosensor, or by surface plasmon resonance, e.g., BIACORE™, or by solution-affinity ELISA. “Anti-EGFR” refers to an antigen-binding protein (or other molecule such as an antigen-binding arm), for example an antibody or antigen-binding fragment thereof, that binds specifically to EGFR. “Anti-CD28” refers to an antigen-binding protein (or other molecule such as an antigen-binding arm), for example an antibody or antigen-binding fragment thereof, that binds specifically to CD28. “EGFRxCD28” refers to refers to an antigen-binding protein (or other molecule), for example an antibody or antigen-binding fragment thereof, that binds specifically to EGFR and to CD28 (and, optionally, to one or more other antigens). “Anti-PD-1” refers to an antigen-binding protein (or other molecule such as an antigen-binding arm), for example an antibody or antigen-binding fragment thereof, that binds specifically to PD-1. An isolated antibody or antigen-binding fragment that specifically binds a human protein, e.g., PD-1, may, however, have cross-reactivity to other antigens, such as proteins, e.g., a PD-1 protein, from other (non-human) species.

The present disclosure includes methods of treating cancer comprising administering antigen-binding proteins, e.g., antibodies or antigen-binding fragments, that bind to the same EGFR and CD28 epitopes as an antigen-binding protein (e.g., REGN7075 (also referred to herein as bsAb7075); REGN6321 (also referred to herein as bsAb6321); REGN6322 (also referred to herein as bsAb6322); REGN6323 (also referred to herein as bsAb6323). Other anti-EGFR X anti-CD28 antigen-binding proteins may be found in Tables 9A, 9B and 9C. The amino acid sequences of the EGFR HCVR arms of the bispecific antibodies described herein may be found in Table 1, whereas the amino acid sequences of the CD28 HCVR arms of the bispecific antibodies described herein may be found in Table 3. Other EGFR parental antibodies for use in the present disclosure are described in WO2014/004427. The amino acid sequences for the EGFR HCVR arm and the CD28 HCVR arm of REGN7075, REGN6321, REGN6322 and REGN6323 are found in Table 6. The amino acid sequences of the common light chain variable region described in the present disclosure are also found in Table 6.

Typically, an antibody or antigen-binding fragment of the disclosure which is modified in some way retains the ability to specifically bind to EGFR and CD28, e.g., retains at least 10% of its EGFR and CD28 binding activity (when compared to the parental antibody) when that activity is expressed on a molar basis. Preferably, an antibody or antigen-binding fragment of the disclosure retains at least 20%, 50%, 70%, 80%, 90%, 95% or 100% or more of the EGFR and CD28 binding affinity as the parental antibody. It is also intended that an antibody or antigen-binding fragment may include conservative or non-conservative amino acid substitutions (referred to as “conservative variants” or “function conserved variants” of the antibody) that do not substantially alter its biologic activity.

A “variant” of a polypeptide, such as an immunoglobulin chain (e.g., REGN7075 (also referred to herein as bsAb7075); REGN6321 (also referred to herein as bsAb6321); REGN6322 (also referred to herein as bsAb6322); REGN6323 (also referred to herein as bsAb6323) V_{H}, V_{L}, HC or LC or CDR thereof comprising the amino acid sequence specifically set forth herein), refers to a polypeptide comprising an amino acid sequence that is at least about 70-99.9% (e.g., at least 70, 72, 74, 75, 76, 79, 80, 81, 82, 83, 84, 85, 86, 87, 88, 89, 90, 91, 92, 93, 94, 95, 96, 97, 98, 99, 99.5 or 99.9%) identical or similar to a referenced amino acid sequence that is set forth herein (e.g., any of SEQ ID NOs: 6; 8; 10; 12; 14; 16; 18; 20; 22; 24; 26; 28; 30; 32; 34; 36; 38; 40; 42; 44; 46; 48; 50; 52; 54; 56; or 58-68); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment).

Moreover, a variant of a polypeptide may include a polypeptide such as an immunoglobulin chain (e.g., REGN7075 (also referred to herein as bsAb7075); REGN6321 (also referred to herein as bsAb6321); REGN6322 (also referred to herein as bsAb6322); REGN6323 (also referred to herein as bsAb6323) V_{H}, V_{L}, HC or LC or CDR thereof) which may include the amino acid sequence of the reference polypeptide whose amino acid sequence is specifically set forth herein but for one or more (e.g., 1, 2, 3, 4, 5, 6, 7, 8, 9 or 10) mutations, e.g., one or more missense mutations (e.g., conservative substitutions), non-sense mutations, deletions, or insertions. For example, the present disclosure includes CD28xEGFR antigen-binding proteins which include an EGFR binding arm immunoglobulin light chain (or V_{L}) variant comprising the amino acid sequence set forth in SEQ ID NO: 16 but having one or more of such mutations and/or an immunoglobulin heavy chain (or V_{H}) variant comprising the amino acid sequence set forth in SEQ ID NO: 2 but having one or more of such mutations. In one embodiment, a CD28xEGFR antigen-binding protein includes an immunoglobulin light chain variant comprising LCDR1, LCDR2 and LCDR3 wherein one or more (e.g., 1 or 2 or 3) of such CDRs has one or more of such mutations (e.g., conservative substitutions) and/or an immunoglobulin heavy chain variant comprising HCDR1, HCDR2 and HCDR3 wherein one or more (e.g., 1 or 2 or 3) of such CDRs has one or more of such mutations (e.g., conservative substitutions).

A “conservatively modified variant” or a “conservative substitution”, e.g., of an immunoglobulin chain set forth herein, refers to a variant wherein there is one or more substitutions of amino acids in a polypeptide with other amino acids having similar characteristics (e.g. charge, side-chain size, hydrophobicity/hydrophilicity, backbone conformation and rigidity, etc.). Such changes can frequently be made without significantly disrupting the biological activity of the antibody or fragment. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4^{th} Ed.)). In addition, substitutions of structurally or functionally similar amino acids are less likely to significantly disrupt biological activity. The present disclosure includes bispecific EGFRxCD28 antibodies and antigen-binding fragments thereof comprising such conservatively modified variant immunoglobulin chains.

Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Alternatively, a conservative replacement is any change having a positive value in the PAM250 log-likelihood matrix disclosed in Gonnet et al. (1992) Science 256: 1443-45.

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

As used herein, the term “subject” refers to a mammal (e.g., rat, mouse, cat, dog, cow, sheep, horse, goat, rabbit), preferably a human, for example, in need of prevention and/or treatment of a cancer, e.g., an EGFR-expressing cancer. The subject may have a cancer, e.g., an EGFR-expressing cancer, be predisposed to developing such a condition, and/or would benefit from an inhibition or reduction in EGFR activity or a depletion of EGFR+ cells. In one embodiment, the subject may have, or be at risk of developing, a cancer. In many embodiments, the term “subject” may be used interchangeably with the term “patient.”

As used herein, the expression “a subject in need thereof” means a human or non-human mammal that exhibits one or more symptoms or indications of cancer, and/or who has been diagnosed with cancer, including a solid tumor and who needs treatment for the same.

As used herein, the terms “treat”, “treating”, or the like, mean to alleviate symptoms, eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, and/or to increase duration of survival of the subject.

As used herein, the term “solid tumor” refers to an abnormal mass of tissue that usually does not contain cysts or liquid areas. Solid tumors may be benign (not cancer) or malignant (cancer). For the purposes of the present disclosure, the term “solid tumor” means malignant solid tumors. The term includes different types of solid tumors named for the cell types that form them, viz. sarcomas, carcinomas and lymphomas. However, the term does not include leukemias. In various embodiments, the term “solid tumor” includes cancers arising from connective or supporting tissue (e.g., bone or muscle) (referred to as sarcomas), cancers arising from the body's glandular cells and epithelial cells which line body tissues (referred to as carcinomas), and cancers of the lymphoid organs such as lymph nodes, spleen and thymus (referred to as lymphomas). Lymphoid cells occur in almost all tissues of the body and therefore, lymphomas may develop in a wide variety of organs. In certain embodiments, the term “solid tumor” includes cancers including, but not limited to, colorectal cancer, ovarian cancer, prostate cancer, breast cancer, brain cancer, cervical cancer, bladder cancer, anal cancer, uterine cancer, colon cancer, liver cancer, pancreatic cancer, lung cancer, endometrial cancer, bone cancer, testicular cancer, skin cancer, kidney cancer, stomach cancer, esophageal cancer, head and neck cancer, salivary gland cancer, and myeloma. In certain embodiments, the term “solid tumor” includes cancers including, but not limited to, hepatocellular carcinoma, non-small cell lung cancer, head and neck squamous cell cancer, basal cell carcinoma, breast carcinoma, cutaneous squamous cell carcinoma, chondrosarcoma, angiosarcoma, cholangiocarcinoma, soft tissue sarcoma, colorectal cancer, melanoma, Merkel cell carcinoma, and glioblastoma multiforme. In certain embodiments, the term “solid tumor” comprises more than one solid tumor lesions located separate from one another, e.g., 2, more than 2, more than 5, more than 10, more than 15, more than 20, or more than 25 lesions in a subject in need of treatment. In certain embodiments, the more than one lesions are located distally from one another in the same organ. In certain other embodiments, the tumor lesions may be located in different organs.

As used herein, the expression “in combination with” means that a first therapeutic agent, e.g., a bispecific EGFRxCD28 antibody or antigen-binding fragment thereof, is administered before, after, or concurrent with a second therapeutic agent, e.g., an anti-PD-1 antibody or antigen-binding fragment thereof. The term “in combination with” also includes sequential or concomitant administration of a first therapeutic agent, e.g., a bispecific EGFRxCD28 antibody or antigen-binding fragment thereof and a second therapeutic agent, e.g., an anti-PD-1 antibody or antigen-binding fragment thereof.

### Combination Therapy Methods for Treating or Inhibiting the Growth of Cancers

The present disclosure provides methods for treating, ameliorating or reducing the severity of at least one symptom or indication, or inhibiting the growth of a cancer in a subject, comprising administering an effective dose of a bispecific EGFRxCD28 antibody (e.g., REGN7075; or any combination of anti-EGFR HCVR pairing with an HCVR from any of the CD28 antibodies described herein) to the subject in combination with an effective dose of a PD-1 inhibitor, such as an antibody (e.g., cemiplimab) or antigen-binding fragment thereof.

The combination therapies comprising the bispecific antigen-binding EGFRxCD28 molecules and anti-PD-1 antibodies, or antigen-binding portions thereof, of the disclosure are useful, inter alia, for treating any cancer in which stimulation, activation and/or targeting of an immune response would be beneficial. In particular, the bispecific EGFRxCD28 antibodies or antigen-binding molecules of the present disclosure may be used for the treatment, prevention and/or amelioration of a cancer, e.g., a cancer associated with or mediated by EGFR expression or activity or the proliferation of EGFR+ cells. The mechanisms of action by which the therapeutic methods of the disclosure are achieved include killing of the cells expressing EGFR in the presence of effector cells, for example, T-cells. Cells expressing EGFR which can be inhibited or killed using the antigen-binding molecules of the disclosure include, for example, lung cancer cells.

A cancer, for the purposes herein, refers to a disease characterized by abnormal, excessive and/or uncontrolled cell growth. Exemplary cancers include, but are not limited to, esophageal carcinoma or esophageal cancer, lung squamous cell carcinoma, lung adenocarcinoma, cervical squamous cell carcinoma or cervical carcinoma, glioma, thyroid cancer, lung cancer (e.g., non-small cell lung cancer), colorectal cancer, colon cancer, bladder cancer, rectal cancer, head and neck cancer, stomach cancer, liver cancer, pancreatic cancer, renal cancer, urothelial cancer, prostate cancer or prostate adenocarcinoma, testis cancer, breast cancer (e.g., ductal or lobular carcinoma), cervical cancer or cervical carcinoma, endometrial cancer, ovarian cancer, gastroesophageal cancer, (e.g., gastroesophageal adenocarcinoma), non-central nervous system (CNS) tumor, melanoma, nasopharyngeal carcinoma, anal carcinoma, mesothelioma, renal cell carcinoma (e.g., chromophobe, clear cell, or papillary), gallbladder/cholangiocarcinoma, pancreatic carcinoma, penile squamous cell carcinoma, or vulvovaginal carcinoma. In one embodiment, the cancer is an EGFR expressing cancer. A wide range of cancers express EGFR. Accordingly, the methods of the present disclosure can be used in treating a wide range of cancers.

Cancer characterized by solid tumor cells or cancerous blood cells, which may be an EGFR-expressing cancer e.g., wherein EGFR expression in the cells of the particular subject to be treated has been confirmed, includes esophageal carcinoma, lung squamous cell carcinoma, lung adenocarcinoma, cervical squamous cell carcinoma, endometrial adenocarcinoma, bladder urothelial carcinoma, lung cancer (e.g., non-small cell lung cancer), colorectal cancer, rectal cancer, endometrial cancer, skin cancer (e.g., head & neck squamous cell carcinoma), brain cancer (e.g., glioblastoma multiforme), breast cancer, gastroesophageal cancer, (e.g., gastroesophageal adenocarcinoma), prostate cancer and/or ovarian cancer.

The methods of the present disclosure may also be used to treat or ameliorate, e.g., primary and/or metastatic tumors arising in the colon, lung, breast, renal cancer, and bladder (or from any cancer discussed herein).

The present disclosure also includes methods for treating or ameliorating residual cancer in a subject. As used herein, the term “residual cancer” means the existence or persistence of one or more cancerous cells in a subject following treatment with an anti-cancer therapy.

In certain embodiments, the methods of the present disclosure may be used to treat subjects that show elevated levels of one or more cancer-associated biomarkers [e.g., programmed death ligand 1 (PD-L1), CA125, CA19-9, prostate-specific antigen (PSA), lactate dehydrogenase, KIT, carcinoembryonic antigen, epidermal growth factor receptor (EGFR), ALK gene rearrangement, or circulating tumor DNA]. For example, the methods of the present disclosure comprise administering a therapeutically effective amount of an anti-PD-1 antibody in combination with a bispecific EGFRxCD28 antibody to a subject with an elevated level of PD-L1 and/or EGFR. In one embodiment, the methods of the present disclosure are used in subjects with cancer that are selected on the basis of PD-L1 expression in cancer tissue. In certain embodiments, the methods of the present are used to treat subjects with a cancer wherein the subjects are selected on the basis of at least 1%, at least 2%, at least 5%, at least 10%, at least 20%, at least 30%, at least 40% or at least 50% PD-L1 expression in cancer tissue and/or immune cells. Methods to determine PD-L1 expression in cancer tissue and/or immune cells are well-known in the art. In certain embodiments, the expression of PD-L1 in tumor tissue is determined by any assay known in the art, for example, by an ELISA assay or by an immunohistochemistry (IHC) assay (e.g., Ventana SP263 assay) or as described in PCT publications WO2016124558 or WO2016191751 or US Patent Application Publication US20160305947. In certain embodiments, the expression of PD-L1 is determined by quantitating RNA expression, for example, by in situ hybridization or by RT-PCR. In certain embodiments, the expression of PD-L1 is determined by imaging with a labeled anti-PD-L1 antibody, for example, by immuno-positron emission tomography or iPET [See, e.g., The Oncologist, 12: 1379 (2007); Journal of Nuclear Medicine, 52(8): 1171 (2011), or U.S. Pat. No. 10,736,976, the entire contents of which are expressly incorporated herein by reference in their entirety].

In an embodiment, the method includes the step of determining whether the cancer in the subject expresses EGFR. If such expression is observed, then the bispecific EGFRxCD28 antibody is administered in combination with an anti-PD-1 antibody or antigen binding fragment thereof. For example, in an embodiment, the method comprises taking a biopsy of the cancer and either determining whether the cells of the cancer express EGFR and, if EGFR expression is present, then administering the bispecific EGFRxCD28 antigen-binding protein in combination with an anti-PD-1 antibody or antigen-binding fragment thereof to the subject. In an embodiment, EGFR expression is tested immunohistochemically (IHC) or by ELISA (enzyme linked immunosorbent assay).

According to certain aspects, the present disclosure provides methods for treating, ameliorating or reducing the severity of at least one symptom or indication, or inhibiting the growth of a cancer, e.g., a cancer which is associated with EGFR expression (e.g., lung cancer), comprising administering one or more of the bispecific EGFRxCD28 antibodies or antigen-binding molecules, e.g., REGN7075, as described herein to a subject in combination with an anti-PD-1 antibody or antigen binding fragment thereof (e.g., cemiplimab), for example, after the subject has been shown to be non-responsive to other types of anti-cancer therapies.

For example, the present disclosure includes methods for treating a cancer, such as lung cancer, comprising administering a bispecific EGFRxCD28 antibody or antigen-binding molecule, e.g., REGN7075, and an anti-PD-1 antibody or antigen-binding fragment thereof, to a subject 1 day, 2 days, 3 days, 4 days, 5 days, 6 days, 1 week, 2 weeks, 3 weeks or 4 weeks, 2 months, 4 months, 6 months, 8 months, 1 year, or more after the subject has received the standard of care for subjects suffering from cancer, e.g., lung cancer.

In certain embodiments, the administration of bispecific EGFRxCD28 antibody or antigen-binding fragment thereof, in combination with anti-PD-1 antibody or antigen-binding fragment thereof, leads to increased tumor regression, tumor shrinkage and/or disappearance. In certain embodiments, the administration of an anti-PD-1 antibody and bispecific EGFRxCD28 antibody leads to delay in tumor growth and development, e.g., tumor growth may be delayed by about 3 days, more than 3 days, about 7 days, more than 7 days, more than 15 days, more than 1 month, more than 3 months, more than 6 months, more than 1 year, more than 2 years, or more than 3 years as compared to an untreated subject or a subject treated with either antibody as monotherapy. In certain embodiments, administration of an anti-PD-1 antibody and bispecific EGFRxCD28 antibody prevents tumor recurrence and/or increases duration of survival of the subject, e.g., increases duration of survival by more than 15 days, more than 1 month, more than 3 months, more than 6 months, more than 12 months, more than 18 months, more than 24 months, more than 36 months, or more than 48 months than an untreated subject or a subject which is administered either antibody as monotherapy. In certain embodiments, administration of the anti-PD-1 antibody and bispecific EGFRxCD28 antibody increases progression-free survival or overall survival. In certain embodiments, administration of an anti-PD-1 antibody and bispecific EGFRxCD28 antibody increases response and duration of response in a subject, e.g., by more than 2%, more than 3%, more than 4%, more than 5%, more than 6%, more than 7%, more than 8%, more than 9%, more than 10%, more than 20%, more than 30%, more than 40% or more than 50% over an untreated subject or a subject which has received either antibody as monotherapy.

In certain embodiments, administration of an anti-PD-1 antibody and bispecific EGFRxCD28 antibody to a subject with a cancer leads to at least 30% or more decrease in tumor cells or tumor size (“partial response”). In certain embodiments, administration of an anti-PD-1 antibody and bispecific EGFRxCD28 antibody to a subject with a cancer leads to complete disappearance of all evidence of tumor cells (“complete response”). In certain embodiments, administration of an anti-PD-1 antibody and bispecific EGFRxCD28 antibody to a subject with a cancer leads to complete or partial disappearance of tumor cells/lesions including new measurable lesions. Tumor reduction can be measured by any of the methods known in the art, e.g., X-rays, positron emission tomography (PET), computed tomography (CT), magnetic resonance imaging (MRI), cytology, histology, or molecular genetic analyses.

In certain embodiments, the methods of the present disclosure comprise administering to a subject in need thereof a therapeutically effective amount of a bispecific EGFRxCD28 antibody in combination with an anti-PD-1 antibody, wherein administration of the combination leads to increased overall survival (OS) or progression-free survival (PFS) of the patient as compared to a patient administered with a standard-of-care (SOC) therapy (e.g., chemotherapy, surgery or radiation). In certain embodiments, the PFS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a patient administered with any one or more SOC therapies. In certain embodiments, the OS is increased by at least one month, at least 2 months, at least 3 months, at least 4 months, at least 5 months, at least 6 months, at least 7 months, at least 8 months, at least 9 months, at least 10 months, at least 11 months, at least 1 year, at least 2 years, or at least 3 years as compared to a patient administered with any one or more SOC therapies.

### (I) Dosages and Timing

An “effective” or “therapeutically effective” dose of an anti-PD-1 antibody or antigen-binding fragment thereof, or a bispecific EGFRxCD28 antibody or antigen-binding fragment thereof, e.g., REGN7075, for treating or preventing a cancer, such as an EGFR-expressing cancer, is the amount of the antibody or antigen-binding fragment sufficient to alleviate one or more signs and/or symptoms of the disease in the treated subject, whether by inducing the regression or elimination of such signs and/or symptoms or by inhibiting the progression of such signs and/or symptoms.

The dose of antigen-binding molecule administered to a subject may vary depending upon the age and the size of the subject, target disease, conditions, route of administration, and the like. The preferred dose is typically calculated according to body weight or body surface area. Depending on the severity of the condition, the frequency and the duration of the treatment can be adjusted.

In some embodiments of the disclosure, a therapeutically effective dose of a bispecific EGFRxCD28 antibody, e.g., REGN7075, is 0.1-3000 mg. The dose amount may vary depending upon the age and the size of a subject to be administered, target disease, conditions, route of administration, and the like. In some embodiments, the bispecific EGFRxCD28 antibody is administered at a dose of about 0.1-900 mg, 1-1500 mg, 100-1200 mg, 300-1000 mg, 500-1500 mg, 800-1000 mg, 800-1500 mg, 300-2000 mg, or 300-3000 mg. In some embodiments, the bispecific EGFRxCD28 antibody is administered at a dose of about 0.01 mg, 0.03 mg, 0.05 mg, 0.1 mg, 0.3 mg 0.5 mg, 1 mg, 2 mg 3 mg, 4 mg, 5 mg, 6 mg, 8 mg, 10 mg, 15 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1000 mg, 1200 mg, 1500 mg, 1800 mg, 2000 mg, 2400 mg, 2700 mg, or 3000 mg. In some embodiments, the bispecific EGFRxCD28 antibody is administered at a dose of about 0.1-50 mg/kg, 1-45 mg/kg, 5-10 mg/kg, 10-30 mg/kg, 15-25 mg/kg, 20-30 mg/kg, or 25-40 mg/kg of the subject's body weight. In some embodiments, the bispecific EGFRxCD28 antibody is administered at a dose of about 0.1 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg 15 mg/kg, 20 mg/kg, 25 mg/kg, 30 mg/kg, 35 mg/kg, 45 mg/kg, or 50 mg/kg.

In some embodiments, the bispecific EGFRxCD28 antibody is administered every week. In some embodiments, the bispecific EGFRxCD28 antibody is administered every two weeks. In some embodiments, the bispecific EGFRxCD28 antibody is administered every three weeks.

In some embodiments, the bispecific EGFRxCD28 antibody is administered intravenously (IV). In some embodiments, the IV infusion occurs over about 60 minutes. In some embodiments, the bispecific EGFRxCD28 antibody is administered subcutaneously.

In some embodiments of the disclosure, a therapeutically effective dose of a bispecific EGFRxCD28 antibody, e.g., REGN7075, is 0.1-3000 mg IV or SC every week or every three weeks.

In some embodiments of the disclosure, a therapeutically effective dose of an anti-PD-1 antibody or antigen-binding fragment thereof, e.g., cemiplimab, is 50-1500 mg, e.g., 350 mg. The dose amount may vary depending upon the age and the size of a subject to be administered, target disease, conditions, route of administration, and the like. In some embodiments of the disclosure, a therapeutically effective dose of an anti-PD-1 antibody or antigen-binding fragment thereof, e.g., cemiplimab, is about 50-1500 mg, 100-1250 mg, 150-1000, 200-750 mg, 300-500 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg or 1500 mg. In some embodiments, the anti-PD-1 antibody or antigen-binding fragment thereof is administered at a dose of about 0.1-20 mg/kg, 0.5-15 mg/kg, 1-12 mg/kg, 2- 10 mg/kg, 5-10 mg/kg, or 7.5-10 mg/kg of the subject's body weight. In some embodiments, the anti-PD-1 antibody or antigen-binding fragment thereof is administered at a dose of about 0.1 mg/kg, 0.2 mg/kg, 0.3 mg/kg, 0.4 mg/kg 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg 15 mg/kg, or 20 mg/kg.

In some embodiments, the anti-PD-1 antibody or antigen-binding fragment thereof is administered every week. In some embodiments, the anti-PD-1 antibody or antigen-binding fragment thereof is administered every two weeks. In some embodiments, the anti-PD-1 antibody or antigen-binding fragment thereof is administered every three weeks.

In some embodiments, the anti-PD-1 antibody or antigen-binding fragment thereof is administered intravenously (IV). In some embodiments, the IV infusion occurs over about 30 minutes or about 60 minutes. In some embodiments, the anti-PD-1 antibody or antigen-binding fragment thereof is administered subcutaneously.

In some embodiments, a therapeutically effective dose of an anti-PD-1 antibody or antigen-binding fragment thereof, e.g., cemiplimab, is 50-1500 mg, e.g., 350 mg, intravenously (IV) or subcutaneously (SC) every week (QW), Q2W (every two weeks), or Q3W (every 3 weeks).

In some embodiments, the bispecific EGFRxCD28 antibody or antigen-binding molecule is administered concurrently with the anti-PD-1 antibody or antigen-binding portion thereof. In some embodiments, the bispecific EGFRxCD28 antibody or antigen-binding molecule is administered the same day as the anti-PD-1 antibody or antigen-binding portion thereof. In some embodiments, the bispecific EGFRxCD28 antibody or antigen-binding molecule is administered prior to administration the anti-PD-1 antibody or antigen-binding portion thereof, e.g., 1 hour prior, 2 hours prior, 3 hours prior, 4 hours prior, 5 hours prior, 6 hours prior, 12 hours prior, 1 day prior, 2 days prior, 3 days prior, 4 days prior, 5 days prior, 6 days prior, 7 days prior, 8 days prior, 9 days prior, 10 days prior, 11 days prior, 12 days prior, 13 days prior, 14 days prior, 15 days prior, 16 days prior, 17 days prior, 18 days prior, 19 days prior, 20 days prior, or 21 days prior. In some embodiments, the anti-PD-1 antibody or antigen-binding molecule is administered prior to administration the bispecific EGFRxCD28 antibody or antigen-binding portion thereof, e.g., 1 hour prior, 2 hours prior, 3 hours prior, 4 hours prior, 5 hours prior, 6 hours prior, 12 hours prior, 1 day prior, 2 days prior, 3 days prior, 4 days prior, 5 days prior, 6 days prior, 7 days prior, 8 days prior, 9 days prior, 10 days prior, 11 days prior, 12 days prior, 13 days prior, 14 days prior, 15 days prior, 16 days prior, 17 days prior, 18 days prior, 19 days prior, 20 days prior, or 21 days prior.

In certain embodiments, an initial dose may be followed by administration of a second or a plurality of subsequent doses of antigen-binding protein(s) in an amount that can be approximately the same or less or more than that of the initial dose, wherein the subsequent doses are separated by, e.g., about 1 week, 2 weeks, 3 weeks, 10 days, 20 days or 30 days.

Multiple doses of an antigen-binding molecule (e.g., a bispecific antigen-binding molecule that specifically binds EGFR and CD28 or an anti-PD-1 antibody or antigen-binding fragment thereof) may be administered to a subject over a defined time course. The methods according to this aspect of the disclosure comprise sequentially administering to a subject multiple doses of an antigen-binding molecule of the disclosure. As used herein, “sequentially administering” means that each dose of an antigen-binding molecule is administered to the subject at a different point in time, e.g., on the same day separated by a predetermined interval (e.g., hours), or on different days separated by a predetermined interval (e.g., hours, days, weeks or months). The present disclosure includes methods which comprise sequentially administering to the subject a single initial dose of an antigen-binding molecule, followed by one or more secondary doses of the same or a different antigen-binding molecule, and optionally followed by one or more tertiary doses of the antigen-binding molecule.

The terms “initial dose,” “secondary doses,” and “tertiary doses,” refer to the temporal sequence of administration of the antigen-binding molecule of the disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the antigen-binding molecule, but may differ from one another in terms of frequency of administration. In certain embodiments, however, the amount of an antigen-binding molecule contained in the initial, secondary and/or tertiary doses varies from one another (e.g., adjusted up or down as appropriate) during the course of treatment. In certain embodiments, two or more doses are administered at the beginning of the treatment regimen as “loading doses” followed by subsequent doses that are administered on a less frequent basis (e.g., “maintenance doses”).

In one exemplary embodiment of the present disclosure, each secondary and/or tertiary dose is administered from one to several weeks after the immediately preceding dose. The phrase “the immediately preceding dose,” as used herein, means, in a sequence of multiple administrations, the dose of antigen-binding molecule which is administered to a subject prior to the administration of the very next dose in the sequence with no intervening doses.

The methods according to this aspect of the disclosure may comprise administering to a subject any number of secondary and/or tertiary doses of an antigen-binding molecule (e.g., a bispecific antigen-binding molecule that specifically binds EGFR and CD28 and/or an anti-PD-1 antibody or antigen-binding fragment thereof). For example, in certain embodiments, only a single secondary dose is administered to the subject. In other embodiments, two or more secondary doses are administered to the subject. Likewise, in certain embodiments, only a single tertiary dose is administered to the subject. In other embodiments, two or more tertiary doses are administered to the subject.

In embodiments involving multiple secondary doses, each secondary dose may be administered at the same frequency as the other secondary doses. Similarly, in embodiments involving multiple tertiary doses, each tertiary dose may be administered at the same frequency as the other tertiary doses. Alternatively, the frequency at which the secondary and/or tertiary doses are administered to a subject can vary over the course of the treatment regimen. The frequency of administration may also be adjusted during the course of treatment by a physician depending on the needs of the individual subject following clinical examination.

In some embodiments, a bispecific EGFRxCD28 antibody, e.g., REGN7075, or antigen-binding fragment thereof, is administered every week, every 2 weeks, every 3 weeks, every 10 days, every 20 days, every 30 days, every month, every 2 months, or every 3 months during the course of treatment. In some embodiments, an anti-PD-1 antibody or antigen-binding fragment thereof, e.g., cemiplimab, is administered every week, every 2 weeks, every 3 weeks, every 10 days, every 20 days, every 30 days, every month, every 2 months, or every 3 months during the course of treatment.

In certain aspects, the method of the disclosure comprises the steps of: (i) administering to the subject the bispecific EGFRxCD28 antibody or antigen-binding fragment thereof at a dose of 0.1 mg to 3000 mg subcutaneously or intravenously once every week or once every 3 weeks for a period of monotherapy, wherein the period of monotherapy is at least 3 weeks; and (ii) administering to the subject the bispecific EGFRxCD28 antibody or antigen-binding fragment thereof at a dose of 0.1 mg to 3000 mg subcutaneously or intravenously once every 3 weeks, and administering to the subject an anti-PD-1 antibody or antigen-binding fragment thereof at a dose of 150 mg to 500 mg intravenously or subcutaneously once every 3 weeks. In some embodiments, the period of monotherapy is at least 3 weeks, at least 4 weeks, at least 5 weeks, or at least 6 weeks. In some embodiments, the period of monotherapy is less than a year, less than 9 months, less than 6 months, less than 3 months, less than 6 weeks, or less than 1 month. In some embodiments, the period of monotherapy is at least 3 weeks but less than 1 year.

In some embodiments, during step (ii), the anti-PD-1 antibody or antigen-binding fragment thereof is administered on a different day as the bispecific EGFRxCD28 antibody or antigen-binding fragment thereof. In some embodiments, during step (ii), the anti-PD-1 antibody or antigen-binding fragment thereof is administered on the same day as the bispecific EGFRxCD28 antibody or antigen-binding fragment thereof.

In some embodiments, the bispecific EGFRxCD28 antibody is administered before, at the same time, or after administration of the anti-PD-1 antibody or antigen-binding fragment thereof to the subject. In some embodiments, the anti-PD-1 antibody or antigen-binding fragment thereof is administered before, at the same time, or after administration of the bispecific EGFRxCD28 antibody or antigen-binding fragment thereof to the subject.

In some embodiments, the bispecific EGFRxCD28 antibody is administered at least 2 times, 3 times, 4 times or 5 times or more as a monotherapy first, then the anti-PD-1 antibody or antigen-binding fragment thereof is administered. As a non-limiting example, the bispecific EGFRxCD28 antibody or fragment thereof may be administered at a dose of 0.1 mg to 3000 mg every week (QW) or every 3 weeks (Q3W) by IV infusion or by subcutaneous injection for at least one week prior to administration of an anti-PD-1 antibody or antigen-binding fragment thereof at a dose of 350 mg every 3 weeks (Q3W) by IV infusion or by subcutaneous injection.

### (II) Routes of Administration

The present disclosure provides methods for administering a bispecific EGFRxCD28 antibody, e.g., REGN7075; REGN6321; REGN6322; REGN6323; or any combination of anti-EGFR HCVR pairing with an HCVR from any of the CD28 antibodies described herein, or a pharmaceutical composition thereof, alone or in combination with an anti-PD-1 antibody, e.g., cemiplimab, or antigen-binding fragment thereof to a subject (e.g., a human for example, who suffers from a cancer), comprising introducing the antigen-binding protein or pharmaceutical composition into the body of the subject (e.g., a human), for example, intravenously or subcutaneously. For example, the method comprises piercing the body of the subject with a needle of a syringe and injecting the antigen-binding protein or pharmaceutical composition into the body of the subject, e.g., into the vein, artery, skin, tumor, muscular tissue or subcutis of the subject.

The mode of administration of an antibody or a pharmaceutical composition thereof can vary. Routes of administration include parenteral, non-parenteral, oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, intraocular, intravitreal, transdermal or intra-arterial.

In some embodiments, the bispecific EGFRxCD28 antibody or antigen-binding portion thereof is administered intravenously, and the anti-PD-1 antibody or antigen-binding portion thereof is administered subcutaneously. In some embodiments, the bispecific EGFRxCD28 antibody or antigen-binding portion thereof is administered subcutaneously, and the anti-PD-1 antibody or antigen-binding portion thereof is administered intravenously. In some embodiments, the bispecific EGFRxCD28 antibody or antigen-binding portion thereof is administered intravenously, and the anti-PD-1 antibody or antigen-binding portion thereof is administered intravenously. In some embodiments, the bispecific EGFRxCD28 antibody or antigen-binding portion thereof is administered subcutaneously, and the anti-PD-1 antibody or antigen-binding portion thereof is administered subcutaneously.

In some further embodiments, the bispecific EGFRxCD28 antibody and/or the anti-PD-1 antibody is administered into the body of the subject over about 10-120, 20-100, 30-90 or 45-75 minutes. In some embodiments, the bispecific EGFRxCD28 antibody and/or the anti-PD-1 antibody is administered into the body of the subject over about 15, 20, 25, 30, 35, 40, 45, 50, 55, 60, 65, 70, 75, 80, 85 or 90 minutes.

The present disclosure also provides a vessel (e.g., a plastic or glass vial or ampule, e.g., with a cap or a chromatography column, hollow bore needle or a syringe cylinder) comprising a bispecific EGFRxCD28 antigen-binding protein of the present disclosure or a pharmaceutical composition thereof.

The present disclosure also provides an injection device comprising one or more antigen-binding proteins (e.g., antibody or antigen-binding fragment) that bind specifically to EGFR and CD28 (EGFRxCD28) or a pharmaceutical formulation thereof. The injection device may be packaged into a kit. An injection device is a device that introduces a substance into the body of a subject via a parenteral route, e.g., intramuscular, subcutaneous or intravenous. For example, an injection device may be a syringe or an auto-injector (e.g., pre-filled with the pharmaceutical formulation) which, for example, includes a cylinder or barrel for holding fluid to be injected (e.g., comprising the antibody or fragment or a pharmaceutical formulation thereof), a needle for piecing skin, blood vessels or other tissue for injection of the fluid; and a plunger for pushing the fluid out of the cylinder and through the needle bore and into the body of the subject.

A pre-filled syringe is a syringe which has been filled with a composition (e.g. a pharmaceutical composition comprising a multispecific antigen-binding protein and a pharmaceutically acceptable carrier) prior to sale or transfer to an end-user, e.g., a physician or care-giver, who is to administer the composition to a subject.

Pharmaceutical compositions are described in more detail below.

### (III) Subject Selection

In some embodiments, the methods described herein further comprise one or more steps of selecting a subject. The patient may be selected, e.g., based on inclusion critera, or may be excluded, e.g., based on exclusion criteria. Inclusion and exclusion criteria are described in more detail in Example 2, below.

In some embodiments, the method comprises selecting a subject with an advanced solid tumor.

In some embodiments, the subject has at least one of the following criteria, or is selected on the basis of at least one of the following criteria: (1) has metastatic disease, or locally advanced disease that is not a candidate for curative surgery or curative radiation; (2) is not a candidate for an approved indication of an anti-PD-1 or PD-L1 therapy, or such therapy is otherwise not available to the subject (alone or in combination); (3) has exhausted all therapeutic options that are expected to provide meaningful clinical benefit, either through disease relapse, treatment refractory disease, or intolerance except subjects with malignancies where anti-PD-1/PD-L1 therapies have demonstrated clinical benefit; and/or (4) has any of the following cancer types: (a) colorectal cancer that is microsatellite stable as documented by local pathology; (b) gastric or gastroesophageal junction cancer; (c) esophageal cancer; (d) breast cancer (ductal or lobular carcinoma, regardless of receptor status); (e) non-small-cell lung cancer (NSCLC) (any PD-L1 expression); (f) head and neck squamous cell carcinoma (SCC); (g) nasopharyngeal carcinoma; (h) cervical carcinoma; (i) anal carcinoma; (j) mesothelioma; (k) prostate adenocarcinoma; (I) renal cell carcinoma (chromophobe, clear cell, or papillary); (m) gallbladder/cholangiocarcinoma; (n) urothelial carcinoma; (o) pancreatic carcinoma; (p) penile SCC; (q) vulvovaginal carcinoma; or (r) additional non-CNS tumor types for which elevated EGFR expression in the tumor is demonstrated.

In some embodiments, the method comprises selecting a subject that has been treated with a prior therapy. In one embodiment, the prior therapy is radiation, surgery, chemotherapy, a PD-1 inhibitor, a PD-L1 inhibitor, an anti-VEGF therapy, CAR-T therapy, and/or an anti-EGFR therapy. In some embodiments, the method comprises selecting a subject that has not received prior anti-PD-1 therapy or anti-PD-L1 therapy.

In some embodiments, the method comprises selecting a subject that has microsatellite-stable colorectal cancer (MSS CRC). In some embodiments, the subject with microsatellite-stable colorectal cancer has, or is selected on the basis of, at least one of the following attributes: (a) has metastatic CRC; (b) is not a candidate for curative surgery or curative radiation; (c) may have active metastases are present in the liver and/or peritoneum at the time of screening; (d) has microsatellite stable as documented by pathology report; (e) has received at least one line of therapy in the relapsed/metastatic setting, wherein the therapy comprises anti-EGFR therapy or anti-VEGF therapy; and/or (f) is anti-PD-1/PD-L1 naïve, defined as never having previously been treated with a drug that targets PD-1.

In some embodiments, the method comprises selecting a subject that has microsatellite-stable colorectal cancer (MSS CRC). In some embodiments, the subject with microsatellite-stable colorectal cancer has, or is selected on the basis of, at least one of the following attributes: (a) has metastatic CRC; (b) is not a candidate for curative surgery or curative radiation; (c) no active metastases have been identified in the liver or peritoneum at the time of screening; (d) only sites of disease are present in lung(s) and/lung or lymph nodes; (e) is microsatellite stable as documented by pathology report; (f) has received at least one line of therapy in the relapsed/metastatic setting, wherein the therapy comprises anti-EGFR therapy or anti-VEGF therapy; and/or (g) is anti-PD-1/PD-L1 naïve, defined as never having previously been treated with a drug that targets PD-1.

In some embodiments, the method comprises selecting a subject with triple negative breast cancer (TNBC). In some embodiments, the subject with TNBC has, or is selected on the basis of, at least one of the following attributes: (a) has metastatic TNBC; (b) is not a candidate for curative surgery or curative radiation; (c) is not a candidate for anti-PD-1 or anti-PD-L1 therapy in an approved indication, or such therapy is otherwise not available to the subject; (d) has triple negative cancer (ER-/PR-/Her2-), as documented by pathology report; and/or (e) is anti-PD-1/PD-L1 naïve, defined as never having previously been treated with a drug that targets PD-1.

In some embodiments, the method comprises selecting a subject that cutaneous squamous cell carcinoma (CSCC). In one embodiment, the subject is not a candidate for curative surgery or curative radiation. In one embodiment, the subject is anti-PD-1/PD-L1 naïve, defined as never having previously been treated with a drug that targets PD-1.

In some embodiments, the method comprises selecting a subject with non-small cell lung cancer (NSCLC). In some embodiments, the subject has, or is selected on the basis of, at least one of the following attributes: (a) has advanced or metastatic NSCLC; (b) is not a candidate for curative surgery or curative radiation; (c) does not have targetable molecular alterations (e.g., ALK, ROS1, EGFR, etc.); (d) has received no prior systemic treatment for recurrent or metastatic NSCLC; and/or (e) is anti-PD-1/PD-L1 naïve, defined as never having previously been treated with a drug that targets PD-1.

In some embodiments, the method comprises selecting a subject with previously documented histologically or cytologically documented locally advanced or metastatic EGFR mutated non-squamous NSCLC disease. In some embodiments, the subject has, or is selected on the basis of, at least one of the following attributes: (a) has advanced or metastatic NSCLC; (b) is not a candidate for curative surgery or curative radiation; (c) has a previously documented targetable EGFR mutation (EGFR Exon 19 deletion, EGFR L858R mutation, EGFR exon20 insertion, or exon 18/21 atypical mutations); (d) is chemotherapy naïve; (e) has received treatment with a third generation TKI; and/or (e) is anti-PD-1/PD-L1 naïve, defined as never having previously been treated with a drug that targets PD-1.

In some embodiments, the method comprises selecting a subject with previously documented histologically or cytologically documented locally advanced or metastatic EGFR mutated non-squamous NSCLC disease. In some embodiments, the subject has, or is selected on the basis of, at least one of the following attributes: (a) has advanced or metastatic NSCLC; (b) is not a candidate for curative surgery or curative radiation; (c) has a previously documented targetable EGFR mutation (EGFR Exon 19 deletion, EGFR L858R mutation, EGFR exon20 insertion, or exon 18/21 atypical mutations); (d) has received treatment with platinum-doublet chemotherapy; (e) has received treatment with a third generation TKI; and/or (f) is anti-PD-1/PD-L1 naïve, defined as never having previously been treated with a drug that targets PD-1.

In some embodiments, the method comprises selecting a subject that head and neck squamous cell carcinoma (HNSCC). In some embodiments, the subject has, or is selected on the basis of, at least one of the following attributes: (a) has advanced or metastatic disease; (b) is not a candidate for curative surgery or curative radiation; (c) has PD-L1 expression of CPS ≥1% by a local IHC assay; (d) has received no prior systemic treatment for recurrent or metastatic HNSCC; and/or (e) is anti-PD-1/PD-L1 naïve, defined as never having previously been treated with a drug that targets PD-1.

### (IV) Adverse Events

In some embodiments, the subject develops one or more mild symptoms of an immune-related adverse event after administration of the bispecific EGFRxCD28 antibody, alone or in combination with the anti-PD-1 antibody. In some embodiments, the one or more symptoms of an immune-related adverse event is colitis, diarrhea, hypothyroidism, hyperthyroidism, hypophysitis, adrenal insufficiency, diabetes, hepatitis, neurotoxicity, pneumonitis, renal events, uveitis, myocarditis, pericarditis, or a combination thereof.

In some embodiments, the subject receives one or more additional treatments to treat the one or more symptoms of a mild immune-related adverse event.

In some embodiments, treatment with the bispecific EGFRxCD28 antibody, alone or in combination with the anti-PD-1 antibody, is paused when the subject develops one or more mild symptoms of an immune-related adverse event and resumed when the one or more symptoms resolve.

### Multispecific EGFRxCD28 Antigen-Binding Molecules

The present disclosure provides methods of use of antigen-binding proteins which are multispecific (e.g., bispecific) and bind at least to EGFR and CD28 for treating cancer, in combination with an anti-PD-1 antibody or antigen-binding portion thereof, e.g., cemiplimab. As used herein, such a multispecific antigen-binding protein is referred to in the format AxB, wherein A refers to a binding arm in the multispecific molecule that binds to EGFR and B refers to a binding arm in the multispecific molecule that binds to CD28, or vice versa. EGFRxCD28 or CD28xEGFR refer to a multispecific antigen binding protein that binds to EGFR and CD28. The specific EGFR and CD28 binding arms in a multispecific antigen-binding protein also may be specified in an AxB format wherein A refers to a specific arm and B refers to another specific arm. For example, 085Nx14226P2 refers to a multispecific antigen-binding protein that has the anti-EGFR binding arms of 085N as set forth herein and the anti-CD28 binding arms of 14426P2 as set forth herein. 085N, for example, is a binding arm including the 085N immunoglobulin heavy and light chains or variable regions thereof or CDRs thereof whose sequences are specifically set forth herein or are variants thereof.

Multispecific binding refers to binding to two or more different epitopes (EGFR and CD28 or more) which may be on the same or on different antigens. Multispecific includes bispecific, trispecific and tetraspecific. An antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity.

In certain embodiments, the multispecific antigen-binding proteins comprise bispecific antigen-binding proteins. As used herein, the expression “bispecific antigen-binding protein” means a protein, polypeptide or molecular complex (e.g., antibody or antigen-binding fragment thereof) comprising at least a first antigen-binding domain and a second antigen-binding domain. Each antigen-binding domain within the bispecific antigen-binding molecule comprises at least one CDR that alone, or in combination with one or more additional CDRs and/or FRs, specifically binds to a particular antigen. In the context of the present disclosure, the first antigen-binding domain specifically binds CD28, and the second antigen-binding domain specifically binds a EGFR.

The present disclosure includes methods comprising administration of any of the following multispecific antigen-binding proteins (e.g., bispecific antibodies or antigen-binding fragments thereof): REGN7075, REGN6321, REGN6322, REGN6323, as well as bispecific antibodies prepared by combining any of the EGFR HCVR arms of Table 1 and 8 (e.g., HCVR arms of the parental monoclonal antibodies mAb12999P2, mAb13008P2, mAb35193P2 and mAb13006P2) with any of the CD28 HCVR arms of Table 3 and 8 (e.g., HCVR arms of the parental mAb14226, mAb14193 and mAb14216) and methods of use thereof for treating cancer as set forth herein.

As used herein, the expression “antigen-binding molecule” means a protein, polypeptide or molecular complex comprising or consisting of at least one complementarity determining region (CDR) that alone, or in combination with one or more additional CDRs and/or framework regions (FRs), specifically binds to a particular antigen. In certain embodiments, an antigen-binding molecule is an antibody or a fragment of an antibody, as those terms are defined elsewhere herein.

As used herein, the expression “bispecific antigen-binding molecule” means a protein, polypeptide or molecular complex (e.g. an antibody or antigen-binding fragment thereof) comprising at least a first antigen-binding domain and a second antigen-binding domain. Each antigen-binding domain within the bispecific antigen-binding molecule comprises at least one CDR that alone, or in combination with one or more additional CDRs and/or FRs, specifically binds to a particular antigen. In the context of the present disclosure, the first antigen-binding domain specifically binds a first antigen (e.g., CD28), and the second antigen-binding domain specifically binds a second, distinct antigen (e.g., EGFR). In certain exemplary embodiments of the present disclosure, the bispecific antigen-binding molecule is a bispecific antibody. Each antigen-binding domain of a bispecific antibody comprises a heavy chain variable domain (HCVR) and a light chain variable domain (LCVR).

The first antigen-binding domain and the second antigen-binding domain may be directly or indirectly connected to one another to form a bispecific antigen-binding molecule of the present disclosure. Alternatively, the first antigen-binding domain and the second antigen binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin C_{H}3 domain. A non-limiting example of a multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

Bispecific antigen-binding molecules of the present disclosure will typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of 1 to about 200 amino acids in length containing at least one cysteine residues. In other embodiments, the multimerizing domain is a cysteine residue, or a short cysteine containing peptide. Other multimerizing domains include peptides or polypeptides comprising or consisting of a leucine zipper, a helix-loop motif, or a coiled-coil motif.

Any bispecific antibody format or technology may be used to make the bispecific antigen-binding molecules of the present disclosure. For example, an antibody or antigen-binding fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present disclosure include, without limitation, e.g., scFv-based or diabody bispecific formats, IgG-scFv fusions, dual variable domain (OVO)-Ig, Quadroma, knobs-into-holes, common light chain (e.g., common light chain with knobs-intoholes, etc.), CrossMab, CrossFab, (SEEO)body, leucine zipper, Ouobody, IgG1/IgG2, dual acting Fab (OAF)-IgG, and Mab2 bispecific formats (see, e.g., Klein et al. 2012, mAbs 4:6, 1-11, and references cited therein, for a review of the foregoing formats).

In the context of bispecific antigen-binding molecules of the present disclosure, the multimerizing domains, e.g., Fc domains, may comprise one or more amino acid changes (e.g., insertions, deletions or substitutions) as compared to the wild-type, naturally occurring version of the Fc domain. For example, the disclosure includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications include, e.g., a modification at position 250 (e.g., E or Q); 250 and 428 (e.g., L or F); 252 (e.g., LN/FIW or T), 254 (e.g., S or T), and 256 (e.g., S/R/Q/EID or T); or a modification at position 428 and/or 433 (e.g., UR/S/P/Q or K) and/or 434 (e.g., H/F or V); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252,254, and 256 (e.g., 252Y, 254T, and 256E) modification; a 250Q and 428L modification (e.g., T250Q and M428L); and a 307 and/or 308 modification (e.g., 308F or 308P).

The present disclosure also includes bispecific antigen-binding molecules comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L 18M, N44S, K52N, V57M, and V821 (by IMGT; D356E, L358M, N384S, K392N, V397M, and V4221 by EU) in the case of IgG1 antibodies; N44S, K52N, and V821 (IMGT; N384S, K392N, and V4221 by EU) in the case of IgG2 antibodies; and Q15R, N44S, K52N, V57M, R69K, E79Q, and V821 (by IMGT; Q355R, N384S, K392N, V397M, R409K, E419Q, and V4221 by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]-[IgG4 upper hinge]-[IgG2 lower hinge]-[IgG4 C_{H}2]-[IgG4 C_{H}3]. Another example of a chimeric Fc domain that can be included in any of the antigen-binding molecules set forth herein comprises, from N- to C-terminus: [IgG1 C_{H}1]-[IgG1 upper hinge]-[IgG2 lower hinge]-[IgG4 C_{H}2]-[IgG1 C_{H}3]. These and other examples of chimeric Fc domains that can be included in any of the antigen-binding molecules of the present disclosure are described in WO2014/022540 A1, Chimeric Fc domains having these general structural arrangements, and variants thereof, can have altered Fc receptor binding, which in turn affects Fc effector function.

Antibodies and antigen-binding fragments of the present disclosure comprise immunoglobulin chains including the amino acid sequences specifically set forth herein (and variants thereof) as well as cellular and in vitro post-translational modifications to the antibody or fragment. For example, the present disclosure n includes antibodies and antigen-binding fragments thereof that specifically bind to EGFR and CD28 comprising heavy and/or light chain amino acid sequences set forth herein as well as antibodies and fragments wherein one or more asparagine, serine and/or threonine residues is glycosylated, one or more asparagine residues is deamidated, one or more residues (e.g., Met, Trp and/or His) is oxidized, the N-terminal glutamine is pyroglutamate (pyroE) and/or the C-terminal lysine or other amino acid is missing.

The present disclosure also provides antigen-binding proteins, such as antibodies (e.g., human antibodies, monoclonal antibodies and recombinant antibodies) and antigen-binding fragments thereof, that specifically bind to CD28 protein or an antigenic fragment thereof (e.g., the extracellular domain of CD28). Antigen-binding proteins that bind to the same epitope on CD28 as, or compete for binding to CD28 with any of the antigen-binding proteins set forth herein, are also part of the present disclosure.

The multispecific EGFRxCD28 antigen-binding proteins of the present disclosure which bind to CD28 on the surface of a T-cell and agonize CD28 signaling enhancing activation and/or proliferation of the T-cell may be referred to, herein, as “co-stimulatory” or “costimulatory”. T-cell activation is initiated upon binding of the T-cell Receptor (TCR)/CD3 complex to peptide-MHC complexes (“signal 1”); activation is then enhanced by engagement of a second “co-stimulatory” receptor, such as the CD28 receptor on T-cells binding to its cognate ligand(s) on the target cell (“signal 2”). For example, activation of a T-cell by a CD28 bi-specific antibody may be caused by amplification of signals in response to endogenous tumor antigen recognition by the TCR/CD3 complex, or in response to “signal 1” activation via a CD3-bispecific.

### (I) Antibodies Comprising Fc Variants

According to certain embodiments of the present disclosure, anti-EGFR X anti-CD28 bispecific antigen binding molecules are provided comprising an Fc domain comprising one or more mutations which enhance or diminish antibody binding to the FcRn receptor, e.g., at acidic pH as compared to neutral pH. For example, the present disclosure includes antibodies and antigen binding molecules comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T); 254 (e.g., S or T), and/or 256(e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and/or 434.

In one embodiment, the modification comprises a
428L (e.g., M428L) and 434S (e.g., N434S) modification;
a 428L, 259| (e.g., V2591), and 308F (e.g., V308F) modification;
a 433K (e.g., H433K) and a 434 (e.g., 434Y) modification;
a 252, 254, and 256 (e.g., 252Y, 254T, and 256E) modification;
a 250Q and 428L modification (e.g., T250Q and M428L); and/or
a 307 and/or 308 modification (e.g., 308F or 308P).

For example, the present disclosure includes EGFRxCD28 bispecific antigen binding molecules comprising an Fc domain comprising one or more pairs or groups of mutations selected from the group consisting of:
250Q and 248L (e.g., T250Q and M248L);
252Y, 254T and 256E (e.g., M252Y, S254T and T256E);
428L and 434S (e.g., M428L and N434S); and
433K and 434F (e.g., H433K and N434F).

All possible combinations of the foregoing Fc domain mutations, and other mutations within the antibody variable domains disclosed herein, are contemplated within the scope of the present disclosure.

### Anti-PD-1 Antibodies and Antigen-Binding Fragments thereof

According to certain exemplary embodiments of the present disclosure, the methods comprise administering a therapeutically effective amount of an anti-PD-1 antibody or antigen-binding fragment thereof in combination with a bispecific EGFRxCD28 antigen-binding molecule or antigen-binding fragment thereof. According to certain embodiments, the anti-PD-1 antibodies, or antigen-binding fragments thereof, used in the methods of the present disclosure specifically bind PD-1. For example, an antibody that “specifically binds” PD-1, as used in the context of the present disclosure, includes antibodies or antigen-binding fragments thereof that bind PD-1 or portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody or antigen-binding fragment that specifically binds human PD-1 may, however, have cross-reactivity to other antigens, such as PD-1 molecules from other (non-human) species.

According to certain exemplary embodiments of the present disclosure, the anti-PD-1 antibody, or antigen-binding fragment thereof comprises a heavy chain variable region (HCVR), light chain variable region (LCVR), and/or complementarity determining regions (CDRs) comprising any of the amino acid sequences of the anti-PD-1 antibodies as set forth in U.S. Pat. No. 9,987,500. In certain exemplary embodiments, the anti-PD-1 antibody or antigen-binding fragment thereof that can be used in the context of the methods of the present disclosure comprises the heavy chain complementarity determining regions (HCDRs) of a heavy chain variable region (HCVR) comprising the amino acid sequence of SEQ ID NO: 73 and the light chain complementarity determining regions (LCDRs) of a light chain variable region (LCVR) comprising the amino acid sequence of SEQ ID NO: 74. According to certain embodiments, the anti-PD-1 antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 75; the HCDR2 comprises the amino acid sequence of SEQ ID NO: 76; the HCDR3 comprises the amino acid sequence of SEQ ID NO: 77; the LCDR1 comprises the amino acid sequence of SEQ ID NO: 78; the LCDR2 comprises the amino acid sequence of SEQ ID NO: 79 (AAS); and the LCDR3 comprises the amino acid sequence of SEQ ID NO: 80. In yet other embodiments, the anti-PD-1 antibody or antigen-binding fragment thereof comprises an HCVR comprising SEQ ID NO: 73 and an LCVR comprising SEQ ID NO: 74. In certain embodiments, the methods of the present disclosure comprise the use of an anti-PD-1 antibody, wherein the antibody comprises a heavy chain comprising the amino acid sequence of SEQ ID NO: 81. In some embodiments, the anti-PD-1 antibody comprises a light chain comprising the amino acid sequence of SEQ ID NO: 82. An exemplary antibody comprising a HCVR comprising the amino acid sequence of SEQ ID NO: 73 and a LCVR comprising the amino acid sequence of SEQ ID NO: 74 is the fully human anti-PD-1 antibody known as cemiplimab (REGN2810; LIBTAYO®). According to certain exemplary embodiments, the methods of the present disclosure comprise the use of cemiplimab, or a bioequivalent thereof. The term “bioequivalent”, as used herein, refers to anti-PD-1 antibodies or PD-1-binding proteins or fragments thereof that are pharmaceutical equivalents or pharmaceutical alternatives whose rate and/or extent of absorption do not show a significant difference with that of cemiplimab when administered at the same molar dose under similar experimental conditions, either single dose or multiple doses. In the context of the disclosure, the term refers to antigen-binding proteins that bind to PD-1 which do not have clinically meaningful differences with cemiplimab in their safety, purity and/or potency.

Other anti-PD-1 antibodies that can be used in the context of the methods of the present disclosure include, e.g., the antibodies referred to and known in the art as nivolumab (U.S. Pat. No. 8,008,449), pembrolizumab (U.S. Pat. No. 8,354,509), MEDI0608 (U.S. Pat. No. 8,609,089), BI 754091, spartalizumab (also known as PDR001), camrelizumab (also known as SHR-1210), JNJ-63723283, MCLA-134, toripalimab, sintilimab, tislelizumab, serplulimab, dostarlimab, retifanlimab, zimberelimab, penpulimab, pidilizumab, HX008, balstilimab, ezabenlimab, or any of the anti-PD-1 antibodies as set forth in U.S. Pat. Nos. 6,808,710, 7,488,802, 8,008,449, 8,168,757, 8,354,509, 8,609,089, 8,686,119, 8,779,105, 8,900,587, and 9,987,500, and in patent publications WO 2006/121168, WO 2009/114335; or antigen-binding fragments of any of the foregoing.

### Bioequivalents

The present disclosure encompasses methods comprising administering antigen-binding molecules having amino acid sequences that vary from those of the described antibodies but that retain the ability to bind CD28 and EGFR or PD-1. Such variant molecules comprise one or more additions, deletions, or substitutions of amino acids when compared to parent sequence, but exhibit biological activity that is essentially equivalent to that of the described antigen-binding molecules. Likewise, the antigen binding molecules-encoding DNA sequences of the present disclosure encompass sequences that comprise one or more additions, deletions, or substitutions of nucleotides when compared to the disclosed sequence, but that encode an antigen binding molecule that is essentially bioequivalent to the described antigen-binding molecules of the disclosure. Examples of such variant amino acid and DNA sequences are discussed above.

The present disclosure includes methods comprising administering antigen-binding molecules that are bioequivalent to any of the exemplary antigen-binding molecules set forth herein. Two antigen-binding proteins or antibodies are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a subject can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the exemplary bispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include the exemplary bispecific antigen-binding molecules set forth herein comprising amino acid changes which modify the glycosylation characteristics of the antibodies, e.g., mutations which eliminate or remove glycosylation.

### Pharmaceutical Formulations and Kits

The present disclosure provides compositions that include bispecific EGFRxCD28 antibodies, e.g., REGN7075, and/or anti-PD-1 antibodies or antigen-binding portions thereof, and one or more ingredients; as well as methods of use thereof.

The present disclosure provides pharmaceutical compositions comprising the bispecific EGFRxCD28 antibody or antigen-binding fragment thereof, and/or the anti-PD-1 antibody or antigen-binding fragment thereof, of the present disclosure. The pharmaceutical compositions of the disclosure can be formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

To prepare pharmaceutical formulations of the antigen-binding proteins, e.g., antibodies and antigen-binding fragments thereof (e.g., REGN7075; REGN6321; REGN6322; REGN6323; or cemiplimab, the antigen-binding protein is admixed with a pharmaceutically acceptable carrier or excipient. See, e.g., Remington's Pharmaceutical Sciences and U.S. Pharmacopeia: National Formulary, Mack Publishing Company, Easton, Pa. (1984); Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y. In one embodiment, the pharmaceutical formulation is sterile. Such compositions are part of the present disclosure.

Pharmaceutical formulations of the present disclosure include a bispecific EGFRxCD28 antigen-binding protein, and/or an anti-PD-1 antibody or antigen-binding fragment thereof, and a pharmaceutically acceptable carrier including, for example, water, buffering agents, preservatives and/or detergents.

The scope of the present disclosure includes desiccated, e.g., freeze-dried compositions, comprising a bispecific EGFRxCD28 antigen-binding protein, or an anti-PD-1 antibody or antigen-binding fragment thereof, or a pharmaceutical formulation thereof that includes a pharmaceutically acceptable carrier but substantially lacks water.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

As discussed herein, the present disclosure provides a vessel (e.g., a plastic or glass vial) or injection device (e.g., syringe, pre-filled syringe or autoinjector) comprising any of the antigen-binding proteins herein, e.g., antibodies or antigen-binding fragments thereof, or a pharmaceutical formulation comprising a pharmaceutically acceptable carrier or excipient thereof.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable and disposable pens and autoinjector delivery devices have applications in the subcutaneous delivery of a pharmaceutical composition of the present disclosure. See e.g., AUTOPEN™ (Owen Mumford, Inc., Woodstock, UK) or the HUMIRA™ Pen (Abbott Labs, Abbott Park, IL)

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous,

subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline and other isotonic solutions which may be used in combination with an appropriate solubilizing agent. Injectable oily mediums are also part of the present disclosure. Such oily mediums may be combined with a solublilzing agent.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 0.1 to about 2000 mg per dosage form in a unit dose; especially in the form of injection.

The present disclosure also provides kits comprising a bispecific EGFRxCD28 antibody and an anti-PD-1 antibody, or antigen-binding portions thereof, for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a cancer, the kit comprising: (a) a dosage of an antibody or an antigen-binding portion thereof; and (b) a dosage of a bispecific EGFRxCD28 antibody or an antigen-binding portion thereof; and (c) instructions for using the antibodies in any of the therapeutic methods disclosed herein. In certain embodiments, the dosage of the anti-PD-1 antibody or antigen-binding fragment thereof ranges from 150 to 550 mg, e.g., 350 mg. In certain embodiments, the dosage of the bispecific EGFRxCD28 antibody or antigen-binding fragment thereof ranges from 0.1 mg to 3000 mg.

### Additional Combination Therapies

The present disclosure provides methods of treating and preventing a disease, e.g., a cancer, by administering a bispecific EGFRxCD28 antibody, e.g., REGN7075, or an antigen-binding fragment thereof, alone as a monotherapy or with an anti-PD-1 antibody or antigen-binding fragment thereof as a combination therapy; and optionally with one or more therapeutic agent(s), e.g., at least a third therapeutic agent or therapy.

In certain embodiments, the third therapeutic agent or therapy is selected from surgery, radiation, chemotherapy (e.g., anti-cancer chemotherapy, for example, paclitaxel, docetaxel, vincristine, cisplatin, carboplatin or oxaliplatin), CAR-T cell therapy, a cancer vaccine, an oncolytic virus, a cytokine, anti-VEGF therapy, anti-EGFR therapy or an anti-cancer drug. As used herein, “anti-cancer drug” means any agent useful to treat cancer including, but not limited to, cytotoxins and agents such as antimetabolites, alkylating agents, anthracyclines, antibiotics, antimitotic agents, procarbazine, hydroxyurea, asparaginase, corticosteroids, mytotane (O,P′-(DDD)), biologics (e.g., antibodies and interferons) and radioactive agents. As used herein, “a cytotoxin or cytotoxic agent”, also refers to a chemotherapeutic agent and means any agent that is detrimental to cells. Examples include Taxol® (paclitaxel), temozolamide, cytochalasin B, gramicidin D, ethidium bromide, emetine, cisplatin, mitomycin, etoposide, tenoposide, vincristine, vinbiastine, coichicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In certain embodiments, the methods comprise administering an agent to reduce or ameliorate or treat symptoms of an immune-related adverse event. In one embodiment, the agent is selected from an IL-6 inhibitor (e.g., an anti-IL6 receptor antibody such as tocilizumab or sarilumab), a corticosteroid or a non-steroidal anti-inflammatory agent.

In a further embodiment, a further therapeutic agent that is administered to a subject in combination with a bispecific EGFRxCD28 antibody or antigen-binding fragment thereof and an anti-PD-1 antibody or antigen-binding fragment thereof, is administered to the subject in accordance with the Physicians' Desk Reference 2003 (Thomson Healthcare; 57^{th} edition (Nov. 1, 2002)) or the approved prescribing information normally provided with the particular agent.

The term “in combination with” indicates that components, a bispecific EGFRxCD28 antibody or antigen-binding fragment thereof, e.g., REGN7075, an anti-PD-1 antibody or antigen-binding fragment thereof, e.g., cemiplimab, along with a further therapeutic agent, can be formulated into a single composition, e.g., for simultaneous delivery, or formulated separately into two or more compositions (e.g., a kit including each component). Components administered “in combination with” each another can alternatively be administered to a subject at a different time than when the other component is administered; for example, each administration may be given non-simultaneously (e.g., separately or sequentially) at intervals over a given period of time as part of a treatment regimen. Separate components administered in combination with each another may also be administered sequentially, though essentially simultaneously, during the same administration session. Moreover, the separate components administered in combination with each another may be administered to a subject by the same or by a different route.

In some embodiments, the third therapeutic agent is administered on the same day as the bispecific EGFRxCD28 antibody and/or the anti-PD-1 antibody. In some embodiments, the third therapeutic agent is administered on the same day and after the bispecific EGFRxCD28 antibody and/or the anti-PD-1 antibody. In some embodiments, the third therapeutic agent is administered on the same day and before the bispecific EGFRxCD28 antibody and/or the anti-PD-1 antibody. In some embodiments, the third therapeutic agent is administered on the same day and after the bispecific EGFRxCD28 antibody but on a different day as the anti-PD-1 antibody. In some embodiments, the third therapeutic agent is administered on the same day and before the bispecific EGFRxCD28 antibody but on a different day as the anti-PD-1 antibody. In some embodiments, the third therapeutic agent is administered on the same day and after the anti-PD-1 antibody but on a different day as the bispecific EGFRxCD28 antibody. In some embodiments, the third therapeutic agent is administered on the same day and before the anti-PD-1 antibody but on a different day as the bispecific EGFRxCD28 antibody.

In some embodiments, the third therapeutic agent is administered on a different day as the bispecific EGFRxCD28 antibody and/or the anti-PD-1 antibody. In some embodiments, the third therapeutic agent is administered on a different day and after the bispecific EGFRxCD28 antibody and/or the anti-PD-1 antibody. In some embodiments, the third therapeutic agent is administered on a different day and before the bispecific EGFRxCD28 antibody and/or the anti-PD-1 antibody. In some embodiments, the third therapeutic agent is administered on a different day and after the bispecific EGFRxCD28 antibody but on a different day as the anti-PD-1 antibody. In some embodiments, the third therapeutic agent is administered on a different day and before the bispecific EGFRxCD28 antibody but on a different day as the anti-PD-1 antibody.

In some embodiments the combination therapy with the bispecific EGFRxCD28 antibody, e.g., REGN7075, and the anti-PD-1 antibody, e.g., cemiplimab, is administered alone for a lead-in period before treatment with the third therapeutic begins.

In some embodiments, the combination therapy with the third therapeutic agent is administered at least 2 times, 3 times, 4 times, 5 times, 6 times, 7 times, or more.

In certain embodiments, the subject exhibits no side effects or does not suffer from any side effects from the administration of the bispecific EGFRxCD28 antibody, alone or in combination with the and/or the anti-PD-1 antibody, alone or in combination with the third therapeutic agent.

### EXAMPLES

The following examples are put forth so as to provide those of ordinary skill in the art with a complete disclosure and description of how to make and use the methods and compositions of the disclosure, and are not intended to limit the scope of what the inventors regard as their disclosure.

### Example 1

### Bispecific EGFRxCD28 Antibodies

### Generation of Anti-EGFR and Anti-CD28 Antibodies

Anti-EGFR and anti-CD28 antibodies were obtained as described in WO2020/198009, the entire contents of which are expressly incorporated herein by reference.

Table 1 sets forth the amino acid sequence identifiers of the heavy and light chain variable regions and CDRs of selected anti-EGFR antibodies of the disclosure. The corresponding nucleic acid sequence identifiers are set forth in Table 2.

TABLE 1
Amino Acid Sequence Identifiers for Parental EGFR
Monoclonal Antibodies (mAb)

| | SEQ ID NOS: | | | | | | | |
|---|---|---|---|---|---|---|---|---|
| Antibody | | | | | | | | |
| Designation | HCVR | HCDR1 | HCDR2 | HCDR3 | LCVR | LCDR1 | LCDR2 | LCDR3 |
| mAb12999P2 | 2 | 4 | 6 | 8 | 16 | 18 | 20 | 22 |
| mAb13008P2 | 30 | 32 | 34 | 36 | 16 | 18 | 20 | 22 |
| mAb35193P2 | 40 | 42 | 44 | 46 | 16 | 18 | 20 | 22 |
| mAb13006P2 | 50 | 52 | 54 | 56 | 16 | 18 | 20 | 22 |

SEQ ID NO: 20: Gly Ala Ser (GAS)

TABLE 2
Nucleic Acid Sequence Identifiers for Parental EGFR
Monoclonal Antibodies (mAb)

| | SEQ ID NOS: | | | | | | | |
|---|---|---|---|---|---|---|---|---|
| Antibody | | | | | | | | |
| Designation | HCVR | HCDR1 | HCDR2 | HCDR3 | LCVR | LCDR1 | LCDR2 | LCDR3 |
| mAb12999P2 | 1 | 3 | 5 | 7 | 15 | 17 | 19 | 21 |
| mAb13008P2 | 29 | 31 | 33 | 35 | 15 | 17 | 19 | 21 |
| mAb35193P2 | 39 | 41 | 43 | 45 | 15 | 17 | 19 | 21 |
| mAb13006P2 | 49 | 51 | 53 | 55 | 15 | 17 | 19 | 21 |

SEQ ID NO: 19: ggggcaagt

Table 3 sets forth the amino acid sequence identifiers of the heavy and light chain variable regions and CDRs of selected anti-CD28 antibodies of the disclosure. The corresponding nucleic acid sequence identifiers are set forth in Table 4.

TABLE 3
Amino Acid Sequence Identifiers for Parental CD28 Monoclonal Antibodies (mAb)

| Antibody | SEQ ID NOS: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb14226 | 10 | 12 | 6 | 14 | 16 | 18 | 20 | 22 |
| mAb14193 | 59 | 60 | 61 | 62 | 16 | 18 | 20 | 22 |
| mAb14216 | 63 | 64 | 65 | 66 | 16 | 18 | 20 | 22 |

TABLE 4
Nucleic Acid Sequence Identifiers for Parental CD28 Antibodies

| Antibody | SEQ ID NOS: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb14226 | 9 | 11 | 5 | 13 | 15 | 17 | 19 | 21 |
| mAb14193 | | | | | 15 | 17 | 19 | 21 |
| mAb14216 | | | | | 15 | 17 | 19 | 21 |

Generation of Bispecific Antibodies (bsAbs) that Bind CD28 and EGFR

Bispecific antibodies comprising an anti-EGFR-specific binding domain and an anti-CD28-specific binding domain were constructed using standard methodologies, wherein the anti-EGFR antigen binding domain and the anti-CD28 antigen binding domain each comprise different, distinct HCVRs paired with a common LCVR. In some instances the bispecific antibodies were constructed utilizing a heavy chain from an anti-CD28 antibody, a heavy chain from an anti-EGFR antibody and a common light chain comprising the components, amino acid and nucleic acid sequences encoding the antibodies as shown below in Tables 5, 6, 7 and 8. Additional bispecific antibodies that bind to EGFR and CD28 may be prepared using the parental monoclonal antibodies having the designations shown in Tables 9A, 9B and 9C.

TABLE 5
Summary of Antibody Designations for HCVR Arms
of Anti-EGFR × Anti-CD28 Bispecific Antibodies

| | Anti-EGFR | Anti-CD28 |
|---|---|---|
| | Antigen-Binding | Antigen-Binding |
| Bispecific | Domain (Parental | Domain (Parental |
| Antibody | Antibody | Antibody |
| Designation | Designation) | Designation) |
| bsAb7075 (Also | mAb12999P2 | mAb14226 |
| referred to as | | |
| REGN7075 or | | |
| H4sH24623D) | | |
| bsAb6321 (Also | mAb13008P2 | mAb14226 |
| referred to as | | |
| REGN6321) | | |
| bsAb6322 (Also | mAb35193P2 | mAb14226 |
| referred to as | | |
| REGN6322) | | |
| bsAb6323 (Also | mAb13006P2 | mAb14226 |
| referred to as | | |
| REGN6323) | | |

TABLE 6
Amino Acid Sequences of Anti-EGFR × Anti-CD28 Bispecific Antibodies
Bispecific

| Antibody | Anti-EGFR Antigen-Binding Domain | | | | Anti-CD28 Antigen-Binding Domain | | | | Common Light Chain Variable Region | | | |
|---|---|---|---|---|---|---|---|---|---|---|---|---|
| Designation | HCVR | HCDR1 | HCDR2 | HCDR3 | HCVR | HCDR1 | HCDR2 | HCDR3 | LCVR | LCDR1 | LCDR2 | LCDR3 |
| bsAb7075 | 2 | 4 | 6 | 8 | 10 | 12 | 6 | 14 | 16 | 18 | 20 | 22 |
| bsAb6321 | 30 | 32 | 34 | 36 | 10 | 12 | 6 | 14 | 16 | 18 | 20 | 22 |
| bsAb6322 | 40 | 42 | 44 | 46 | 10 | 12 | 6 | 14 | 16 | 18 | 20 | 22 |
| bsAb6323 | 50 | 52 | 54 | 56 | 10 | 12 | 6 | 14 | 16 | 18 | 20 | 22 |

TABLE 7
Nucleic Acid Sequences Encoding Anti-EGFR × Anti-CD28 Bispecific Antibodies
Bispecific

| Antibody | Anti-EGFR Antigen-Binding Domain | | | | Anti-CD28 Antigen-Binding Domain | | | | Common Light Chain Variable Region | | | |
|---|---|---|---|---|---|---|---|---|---|---|---|---|
| Designation | HCVR | HCDR1 | HCDR2 | HCDR3 | HCVR | HCDR1 | HCDR2 | HCDR3 | LCVR | LCDR1 | LCDR2 | LCDR3 |
| bsAb7075 | 1 | 3 | 5 | 7 | 9 | 11 | 5 | 13 | 15 | 17 | 19 | 21 |
| bsAb6321 | 29 | 31 | 33 | 35 | 9 | 11 | 5 | 13 | 15 | 17 | 19 | 21 |
| bsAb6322 | 39 | 41 | 43 | 45 | 9 | 11 | 5 | 13 | 15 | 17 | 19 | 21 |
| bsAb6323 | 49 | 51 | 53 | 55 | 9 | 11 | 5 | 13 | 15 | 17 | 19 | 21 |

TABLE 8
Amino acid and nucleotide sequences for full length
immunoglobulin chains of bispecific antibodies
bsAb7075, bsAb6321, bsAb6322 and bsAb6323

| Bispecific | | | | | | |
|---|---|---|---|---|---|---|
| Antibody | HC (EGFR) | | HC (CD28) | | LC (EGFR & CD28) | |
| Designation | D | P | D | P | D | P |
| bsAb7075 | 23 | 24 | 25 | 26 | 27 | 28 |
| bsAb6321 | 37 | 38 | 25 | 26 | 27 | 28 |
| bsAb6322 | 47 | 48 | 25 | 26 | 27 | 28 |
| bsAb6323 | 57 | 58 | 25 | 26 | 27 | 28 |

D = Nucleotide sequence of DNA encoding indicated sequence
P = amino acid of polypeptide for the indicated sequence
Numbers refer to SEQ ID NOs for the indicated sequence
HC is the full length heavy chain for the indicated antibody
LC is the full length light chain for the indicated antibody

Additional bispecific antibodies comprising one HCVR from a parental EGFR antibody and the other HCVR arm from a parental CD28 antibody may be made using the techniques described herein. The parental EGFR antibodies used to generate these additional anti-EGFR X anti-CD28 bispecific antibodies have HCVR sequences described in WO2014/004427. The CD28 parental antibodies used to generate these additional anti-EGFR X anti-CD28 bispecific antibodies have the amino acid sequences described above in Table 3. These anti-EGFR and anti-CD28 binding domains (pairings) are shown below in Tables 9A, 9B and 9C.

TABLE 9A
Summary of Parental Antibody Designations for HCVR Arms
of Anti-EGFR × Anti-CD28 Additional Bispecific Antibodies

| Anti-EGFR | Anti-CD28 |
|---|---|
| Antigen-Binding | Antigen-Binding |
| Domain (Parental | Domain (Parental |
| Antibody Designation | Antibody Designation |
| and Sequences found | and Sequences found |
| in WO2014/004427) | herein in Table 3) |
| mAb085N | mAb14226 |
| mAb086N | mAb14226 |
| mAb136P | mAb14226 |
| mAb141P | mAb14226 |
| mAb142P | mAb14226 |
| mAb143P | mAb14226 |
| mAb144P | mAb14226 |
| mAb145P | mAb14226 |
| mAb147P | mAb14226 |
| mAb151P | mAb14226 |
| mAb153P | mAb14226 |
| mAb155P | mAb14226 |
| mAb157P | mAb14226 |
| mAb158P | mAb14226 |
| mAb159P | mAb14226 |
| mAb161P | mAb14226 |
| mAb163P | mAb14226 |
| mAb169P | mAb14226 |
| mAb171P | mAb14226 |

TABLE 9B
Summary of Parental Antibody Designations for HCVR Arms
of Anti-EGFR × Anti-CD28 Additional Bispecific Antibodies

| Anti-EGFR | Anti-CD28 |
|---|---|
| Antigen-Binding Domain | Antigen-Binding Domain |
| (Parental Antibody | (Parental Antibody |
| Designation and Sequences | Designation and Sequences |
| found in WO2014/004427) | found herein in Table 3) |
| mAb085N | mAb14193 |
| mAb086N | mAb14193 |
| mAb136P | mAb14193 |
| mAb141P | mAb14193 |
| mAb142P | mAb14193 |
| mAb143P | mAb14193 |
| mAb144P | mAb14193 |
| mAb145P | mAb14193 |
| mAb147P | mAb14193 |
| mAb151P | mAb14193 |
| mAb153P | mAb14193 |
| mAb155P | mAb14193 |
| mAb157P | mAb14193 |
| mAb158P | mAb14193 |
| mAb159P | mAb14193 |
| mAb161P | mAb14193 |
| mAb163P | mAb14193 |
| mAb169P | mAb14193 |
| mAb171P | mAb14226 |

TABLE 9C
Summary of Parental Antibody Designations for HCVR Arms
of Anti-EGFR × Anti-CD28 Additional Bispecific Antibodies

| Anti-EGFR | Anti-CD28 |
|---|---|
| Antigen-Binding Domain | Antigen-Binding Domain |
| (Parental Antibody | (Parental Antibody |
| Designation and Sequences | Designation and Sequences |
| found in WO2014/004427) | found herein in Table 3) |
| mAb085N | mAb14216 |
| mAb086N | mAb14216 |
| mAb136P | mAb14216 |
| mAb141P | mAb14216 |
| mAb142P | mAb14216 |
| mAb143P | mAb14216 |
| mAb144P | mAb14216 |
| mAb145P | mAb14216 |
| mAb147P | mAb14216 |
| mAb151P | mAb14216 |
| mAb153P | mAb14216 |
| mAb155P | mAb14216 |
| mAb157P | mAb14216 |
| mAb158P | mAb14216 |
| mAb159P | mAb14216 |
| mAb161P | mAb14216 |
| mAb163P | mAb14216 |
| mAb169P | mAb14216 |
| mAb171P | mAb14216 |

Exemplified bispecific antibodies were manufactured having a modified (chimeric) IgG4 Fc domain as set forth in US Patent Application Publication No. US20140243504A1, published on Aug. 28, 2014.

The bispecific antibodies described in this Example comprise two separate antigen-binding domains (i.e., binding arms). The first antigen-binding domain comprises a heavy chain variable region derived from an anti-CD28 antibody (“CD28-VH”), and the second antigen-binding domain comprises a heavy chain variable region derived from an anti-EGFR antibody (“EGFR-VH”). Both the anti-EGFR and the anti-CD28 share a common light chain. The CD28-VH/EGFR-VH pairing creates antigen-binding domains that specifically recognize CD28 on T cells and EGFR on tumor cells.

### Example 2

### Assessment of Treatment with REGN7075 Alone and in Combination with Cemiplimab

This Example describes a phase 1 clinical study of REGN7075 (EGFRxCD28 Costimulatory Bispecific Antibody) in combination with cemiplimab in subjects with advanced solid tumors.

### Study Objectives

Primary Objectives in the Dose Escalation are to assess the safety and tolerability of a REGN7075 monotherapy lead-in and REGN7075 in combination with cemiplimab in subjects with advanced solid tumors.

Primary Objectives in the Dose Expansion are to assess the preliminary efficacy of REGN7075 in combination with cemiplimab within selected advanced solid tumor-specific cohorts, as measured by objective response rate (ORR) per Response Evaluation Criteria in Solid Tumors version 1.1 (RECIST 1.1) and/or composite response criteria (depending on the subject's baseline evaluation criteria).

Secondary Objectives in Dose Escalation are: (i) To characterize the

pharmacokinetics (PK) of REGN7075 alone and in combination with cemiplimab; (ii) To assess the preliminary efficacy of REGN7075 in combination with cemiplimab, as measured by ORR, overall survival (OS), progression free survival (PFS), duration of response (DOR), complete response (CR) rate, and disease control rate (DCR) per RECIST 1.1 and/or composite response criteria (depending on the subject's baseline evaluation criteria); (iii) To assess immunogenicity of REGN7075 and cemiplimab.

Secondary Objectives In Dose Expansion are: (i) To assess the preliminary efficacy of REGN7075 in combination with cemiplimab within selected advanced solid tumor-specific cohorts of subjects, as measured by OS, PFS, DOR, CR rate, and DCR per RECIST 1.1 and/or composite response criteria (depending on the subject's baseline evaluation criteria); (ii) To assess the safety and tolerability of REGN7075 in combination with cemiplimab with or without chemotherapy; (iii) To characterize the PK of REGN7075 alone and in combination with cemiplimab with or without chemotherapy; (iv) To assess immunogenicity to REGN7075 and cemiplimab with or without chemotherapy; (v) To assess the effect of REGN7075 on subject-reported outcomes, including health related quality of life (HRQOL), as measured by the validated instruments European Organisation for Research and Treatment of Cancer Quality of Life Questionnaire (EORTC QLQ-C30), EORTC-QLQ-BR23 (breast cancer patients only), EORTC QLQ-CR29 (among CRC subjects only), EORTC QLQ-LC13 (among NSCLC subjects only) and EORTC QLQ-HN35 (among HNSCC subjects only)and EQ-5D-5L.

Exploratory objectives include: (i) Measurement of circulating tumor DNA levels over time; (ii) To explore tumor gene alterations detected in circulating tumor DNA (ctDNA) at baseline and post-dose associated with efficacy and safety; (iii) To assess the efficacy of REGN7075 alone and in combination with cemiplimab with or without chemotherapy in tumor mutation burden (TMB) low and high patients within selected advanced solid tumor cohorts; and (iv) Assessment of EGFR and programmed cell death ligand (PD-L1) expression in tumor and immune cell populations by evaluating protein and RNA in tumor tissue.

### Study Design

This is an open label, phase 1/2, first in human (FIH) study evaluating safety, tolerability, PK, and preliminary anti-tumor activity of REGN7075 alone and in combination with cemiplimab in patients with advanced solid tumors.

There are two parts of the study:

### Dose Escalation

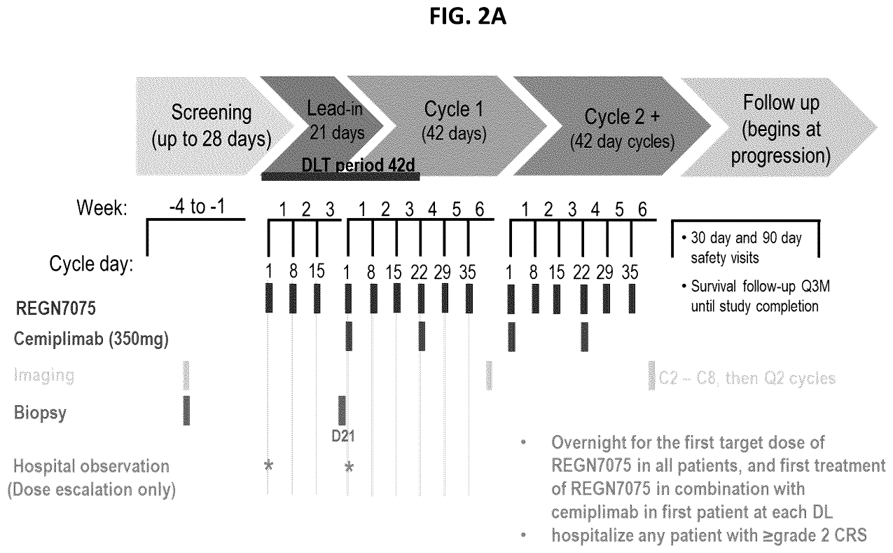

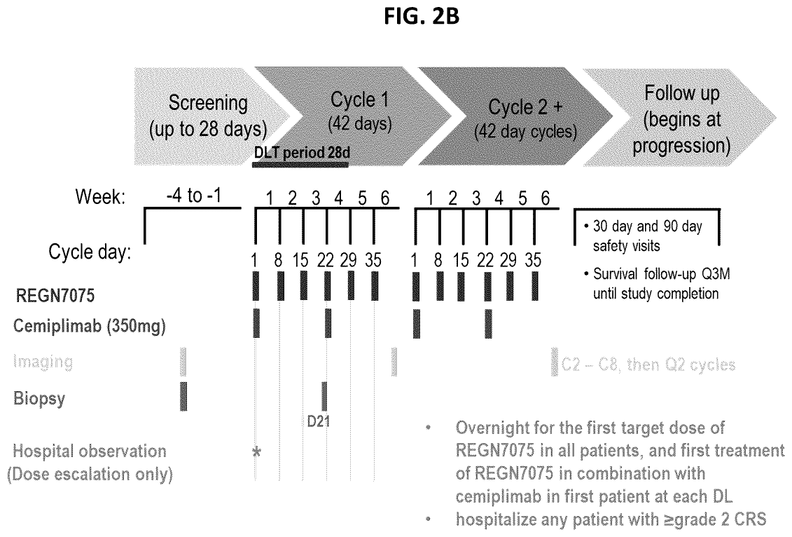

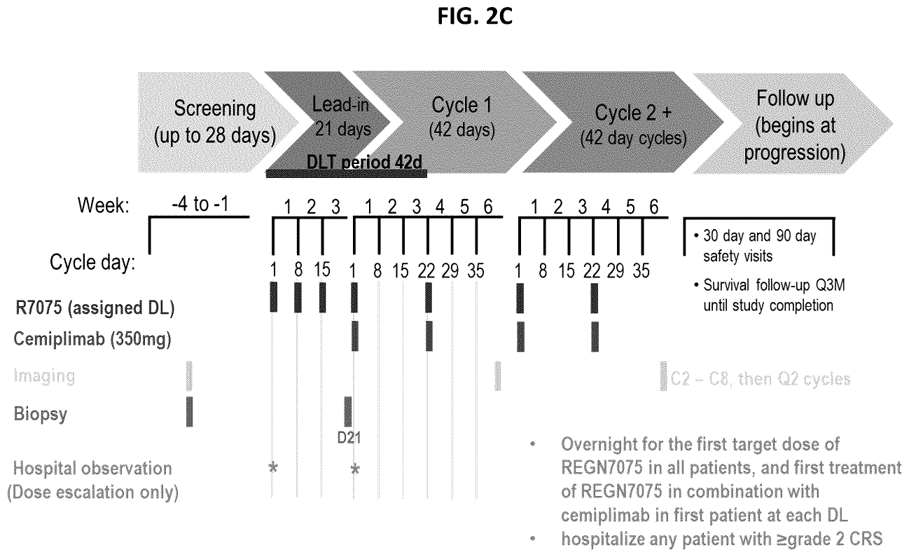

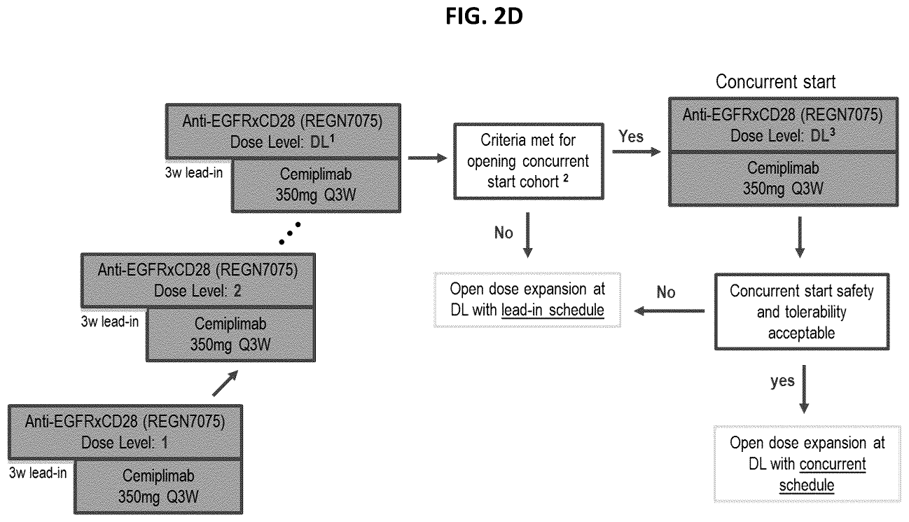

During the dose escalation, patients will receive a 3-week monotherapy lead-in of REGN7075 at the assigned dose level (DL) QW IV followed by combination therapy of REGN7075 at the assigned DL and cemiplimab 350 mg Q3W IV. Dose levels of concurrently started (ie, no lead-in) REGN7075 and cemiplimab 350 mg Q3W IV may be investigated. Once an MTD/RP2D(s) of REGN7075 IV is identified and after enrollment and DLT monitoring are completed for Concurrent Start Dose Escalation cohorts (FIG. 2B), enrollment to exploratory SC dosing cohorts may begin. SC dosing of REGN7075 in combination with either IV or SC cemiplimab may be explored.

Based on emerging PK data, both lead-in and concurrent start dose escalation cohorts may be enrolled with REGN7075 QW (FIGS. 2A and 2B). For management of infusion related reactions (IRR), step up dosing for REGN7075 will include a step-up dose on week 1 and a target dose on week 2 moving forward. The initial step-up dose will be 30 mg. The dose and/or rate of this step-up dose may be adjusted. Additionally, there will be an option to split both step up and first target dose over 2 days.

### Dose Expansion

RP2D(s) will be determined based on the totality of data. Dose expansion will be based on RP2D(s). During dose expansion, patients will receive combination therapy of REGN7075 at the assigned DL (eg, RP2D[s]) and schedule. Dose escalation will consist of patients with a variety of mixed advanced solid tumor types. Dose expansion will consist of the following tumor specific expansion cohorts:
Cohort A: Triple Negative Breast Cancer (TNBC)
Cohort B: Cutaneous Squamous Cell Carcinoma (CSCC)
Cohort C: Non-Small Cell Lung Cancer (NSCLC)
Cohort D: Head and Neck Squamous Cell Carcinoma (HNSCC)
Cohort E: Microsatellite Stable Colorectal Cancer (MSS-CRC), with Active Liver Metastases and/or Active Peritoneal Metastases
Cohort F: MSS-CRC with Isolated Lung/Lymph Node Metastases (no active liver and no active peritoneal metastases)
Cohort G: EGFR-mutant NSCLC Post Third Generation TKI
Cohort H: EGFR-mutant NSCLC Post Third Generation TKI and Post Platinum-Doublet Chemotherapy

Dose expansion cohorts will be enrolled after identification of the REGN7075 RP2D(s) in combination with cemiplimab. Patients in cohorts C and G will also receive 4 cycles of platinum-based chemotherapy. Prior to enrollment of expansion cohorts, additional patients may be enrolled at the presumptive RP2D(s) to further evaluate safety and biologic activity.

Patients will be treated with study drug until unequivocal disease progression, intolerable adverse events (AEs), withdrawal of consent, or other study withdrawal criterion is met.

Treatment will consist of 3-week lead in of REGN7075 at the RP2D(s) QW or concurrent start in combination with cemiplimab 350 mg Q3W IV. Patients in cohorts C and G will also receive 4 cycles of platinum-based chemotherapy, administered Q3W on the same day as cemiplimab.

If the concurrent start schedule (ie, REGN7075 and cemiplimab started without a REGN7075 lead-in period) dose level is not investigated, or the concurrent start dose-level is tested and deemed not tolerable (eg, occurrence of increased cytokine release syndrome (CRS) or other DLTs compared with monotherapy lead in schedule), expansion cohorts will be opened at the previously tolerable dose-level that included a REGN7075 lead in schedule. If the concurrent start dose-level is deemed tolerable, the expansion cohort will be opened and will use the concurrent schedule.

### Study Duration

The total duration for each subject will vary based on the occurrence of 1 or more of the following: disease progression, intolerable AEs, withdrawal of consent, or study withdrawal criterion.

The study timeline consists of: screening (up to 28 days), treatment (variable duration and timing), and follow-up (approximately 90 days from last dose).

The timing of doses is summarized in FIG. 2.

Treatment will continue until either disease progression, intolerable adverse events, withdrawal of consent, or other treatment discontinuation criterion is met. Disease assessment will be performed after cycle 1, after cycle 2, then every 12 weeks thereafter. In the absence of clinical deterioration, there is an option to continue subjects on treatment on the same schedule of events until confirmed radiographic progression (PD on 2 sequential scans; the second scan must occur at least 4 weeks after the first, and must be no later than the subject's next scheduled scan). Further treatment beyond confirmed radiographic progression will be considered upon request, provided treatments remain well tolerated and deemed in the interest of the subject. Response per RECIST 1.1 and/or composite response criteria (depending on the subject's baseline evaluation criteria) will be assessed, but scans will be collected and held with the option for central review.

### Dose Escalation

The study will begin with a dose escalation of REGN7075 in combination with fixed dose cemiplimab in patients with advanced solid tumors that have never been previously treated with an anti-PD-1 or anti-PD-L1 therapy (“anti-PD-1/PD-L1 naïve”). Treatment at each DL (Table 10) consists of a 3-week monotherapy lead-in of REGN7075, followed by combination therapy of REGN7075 with cemiplimab (see FIG. 2A-2D) for a combined DLT period of 6 weeks.

TABLE 10
Planned Dose Escalation cohorts and Enrollment

| | Step up | | | | Initial |
|---|---|---|---|---|---|
| Dose | REGN7075 | | | | Enrollment |
| Level^{1, 2, 3} | (first dose)⁴ | REGN7075 | | Cemiplimab | (N) |
| DL-1⁵ | N/A | 0.01 | mg | 350 mg | 0-4 |
| DL1 | N/A | 0.03 | mg | 350 mg | 1 |
| DL2 | N/A | 0.1 | mg | 350 mg | 1 |
| DL3 | N/A | 0.3 | mg | 350 mg | 1 |
| DL4 | N/A | 1 | mg | 350 mg | 3-4 |
| DL4a | N/A | 2 | mg | 350 mg | 0-4 |
| DL5 | N/A | 3 | mg | 350 mg | 3-4 |
| DL5a | N/A | 6 | mg | 350 mg | 0-4 |
| DL6 | N/A | 10 | mg | 350 mg | 3-4 |
| DL6a | N/A | 20 | mg | 350 mg | 0-4 |
| DL7 | N/A | 30 | mg | 350 mg | 3-4 |
| DL7a | N/A | 60 | mg | 350 mg | 0-4 |
| DL8 | 30 mg | 100 | mg | 350 mg | 3-4 |
| DL8a | 30 mg | 200 | mg | 350 mg | 0-4 |
| DL9 | 30 mg | 300 | mg | 350 mg | 3-4 |
| DL9a | 30 mg | 600 | mg | 350 mg | 0-4 |
| DL10 | 30 mg | 900 | mg | 350 mg | 3-4 |
| DL10a | 30 mg | 1800 | mg | 350 mg | 0-4 |
| DL11 | 30 mg | 2700 | mg | 350 mg | 3-4 |

At the assigned DL, each patient will begin with a REGN7075 monotherapy lead-in during which safety and PK of REGN7075 monotherapy will be assessed. Lead-in is planned to last 21 days and include 3 doses of REGN7075 QW. This will be followed by combination therapy of REGN7075 on either a QW or Q3W schedule (same nominal dose as QW) with cemiplimab 350 mg Q3W. Combination therapy will only begin after the patient, 1) has received at least three doses of REGN7075, and 2) has not developed grade ≥2 CRS, rash, or imAE associated with the most recent REGN7075 monotherapy infusion. If grade ≥2 CRS, rash, or imAE is observed during the third dose of lead-in, but resolves prior to the next scheduled dose of REGN7075, REGN7075 monotherapy will be continued for a fourth dose. If grade ≥2 CRS, rash, or imAE is observed during the fourth dose of monotherapy, patients will not begin combination therapy but will have the option to continue on monotherapy. Monotherapy dosing will be continued weekly as per week 3 (day 15). These patients will be considered uninformative with regard to combination therapy dose selection and may be replaced if necessary but will be allowed to continue on study in monotherapy at the investigator's discretion. In this case, patients would complete all assessments except for cemiplimab treatment. Only when REGN7075 is administered and no grade ≥2 CRS, rash, or imAE occurs will the patient receive the combination of REGN7075 plus cemiplimab. One or more of the specified expansion cohorts may be opened at a chosen dose level that has been shown to be safe while simultaneously continuing with dose escalation.

### Concurrent Start Cohorts

Additional cohorts may be enrolled without a 3-week monotherapy lead-in of REGN7075 (ie, cemiplimab and REGN7075 starting together; herein deemed “concurrent start” schedule). These concurrent start cohorts with REGN7075 dosed QW and/or dosed Q3W with cemiplimab will only be opened after review of the safety and tolerability of REGN7075 monotherapy lead-in and in combination with cemiplimab at the prospective dose.

### Intra-Patient Dose Escalation

Patients with partial response (PR), stable disease (SD) or those with PD that have previously been granted approval to treat beyond progression at or after cycle 3 day 1 (and at least 2 scans), who tolerate therapy (maximum grade ≤2 treatment-related toxicity observed during the previous treatment cycle), but do not demonstrate further improvement at their assigned dose level, may be considered for intra-patient dose escalation (IPDE) with REGN7075 up to the highest DL that has been deemed safe (ie, 1 level below the currently actively enrolling DL if dose escalation is ongoing, or current dose level if dose escalation has been completed and the RP2D(s) has been determined). Patients with PR must demonstrate no further improvement on at least 3 consecutive scans. Patients who continue to tolerate therapy may be considered for further escalation, but no more frequently than once every 6 weeks.

### Subcutaneous Dose Escalation Cohorts

To further inform safety, efficacy, and dosing administration, SC dose escalation cohorts may be initiated. Subcutaneous dose escalation will follow the DL(s) starting at a dose based on preclinical, PK, and clinical data to date. Cemiplimab may continue to be dosed IV (350 mg IV Q3W), or may be administered SC instead (pending results of development of SC cemiplimab in other studies). The SC cohort(s) will be denoted as “SC,” with the milligram dose (eg, if DL8 is a presumptive RP2D(s), then DL_SC_100, where 100 denotes the milligram dose of REGN7075).

### Dose Expansion

Cohorts consisting of a specific solid tumor type will be treated with REGN7075 at a dose and schedule of interest demonstrated to be safe during dose escalation.

If the concurrent start schedule (ie, REGN7075 and cemiplimab started without a REGN7075 lead-in period) dose level is not investigated, or the concurrent start dose-level is tested and deemed not tolerable (eg, occurrence of increased CRS or other DLTs compared with monotherapy lead-in schedule), expansion cohorts will be opened at the previously tolerable dose-level that included a REGN7075 lead-in schedule. If the concurrent start dose-level is deemed tolerable, the expansion cohort will be opened and will use the concurrent schedule. The expansion cohorts are listed below:

### Cohort A: Triple Negative Breast Cancer

Patients with previously documented metastatic TNBC or locally advanced TNBC who are not candidates for curative surgery or curative radiation, and
Are anti-PD-1/PD-L1 naive

### Cohort B: Cutaneous Squamous Cell Carcinoma

Patients with metastatic CSCC or locally advanced CSCC who are not candidates for curative surgery or curative radiation, and
Would otherwise be candidates for cemiplimab monotherapy (or another anti-PD-1/PD-L1 agent), and
Are anti-PD-1/PD-L1 naive

### Cohort C: Non-Small Cell Lung Cancer

Patients with metastatic NSCLC or locally advanced NSCLC who are not candidates for curative surgery or curative radiation, and
Do not have previously documented targetable molecular driver mutations (eg, ALK, ROS1, EGFR, RET-Fusion MET Exon-14 skipping), and
Are anti-PD-1/PD-L1 naïve
Have received no prior systemic treatment for recurrent or metastatic NSCLC (note that adjuvant or neoadjuvant systemic treatments, excluding anti-PD-1/PD-L1 treatment, will not be counted as a prior line).

### Cohort D: Head and Neck Squamous Cell Carcinoma

Patients with metastatic HNSCC or locally advanced HNSCC who are not candidates for curative surgery or curative radiation, and
Patients with previously documented PD-L1 expression of CPS ≥1% by IHC. CPS defined as the number of PD-L1 staining cells (tumor cells, lymphocytes, macrophages) divided by the total number of viable tumor cells, multiplied by 100.
Are anti-PD-1/PD-L1 naïve
   Cohort E: Microsatellite Stable Colorectal Cancer with Active Liver and/or Active Peritoneal Metastases
Patients with metastatic CRC or locally advanced CRC who are not candidates for curative surgery or curative radiation, and
Active metastatic disease in the liver or peritoneum at the time of screening
   Ablated and resected liver disease is not considered active. NOTE: MSS-CRC patients with RECIST evaluable disease that is NOT isolated to lung/lymph node but is not in liver/peritoneum may also enroll in this cohort
Disease is MSS per previously documented result, and
Have received at least one prior line of therapy
   Patients with previously documented RAS wild type disease must have received anti-EGFR therapy or have a documented reason why anti-EGFR therapy was not appropriate
   Patients must have received anti-VEGF therapy or have a documented reason why anti-VEGF therapy was not appropriate
Are anti-PD-1/PD-L1 naïve
   Cohort F: Microsatellite Stable Colorectal Cancer with Isolated Lung/Lymph Node Metastases (No Active Liver and No Active Peritoneal Metastases)
Patients with metastatic CRC or locally advanced CRC who are not candidates for curative surgery or curative radiation, and
No active metastases have been identified in the liver or peritoneum at the time of screening,
   Ablated and resected liver disease is not considered active.
Only sites of metastasis are lung and/or lung lymph nodes
Disease is MSS per previously documented result, and
Have received at least one prior line of therapy
   Patients with previously documented RAS wild type disease must have received anti-EGFR therapy or have a documented reason why anti-EGFR therapy was not appropriate
   Patients must have received anti-VEGF therapy or have a documented reason why anti-VEGF therapy was not appropriate
Are anti-PD-1/PD-L1 naïve

### Cohort G: EGFR-Mutant NSCLC Post 3rd Generation TKI

Patients with histologically or cytologically documented locally advanced or metastatic non-squamous NSCLC who are not candidates for curative surgery or curative radiation, and
Have previously documented targetable EGFR mutations:
   NSCLC that harbors EGFR Exon 19 deletion
   NSCLC that harbors EGFR L858R mutation
   NSCLC with activating EGFR exon20 insertion
   NSCLC with exon 18/21 atypical mutations
   NOTE: EGFR deletion/mutation must have been previously documented by a certified test (either from tissue or ctDNA from blood is allowed)
The documentation of EGFR mutation status can be obtained any time since the initial diagnosis of non-small cell lung cancer.
Are anti-PD1/PD-L1 naïve, and
Have received a third generationTKI treatment
   For patients whose tumors harbor previously documented EFGR Exon19 deletion or L858R mutation, prior osimertinib or other third generation TKI treatment is required
Small cell transformation is excluded

### Cohort H: EGFR-Mutant NSCLC Post 3rd Generation TKI and Post Platinum-Doublet Chemotherapy

Patients with histologically or cytologically documented locally advanced or metastatic non-squamous NSCLC who are not candidates for curative surgery or curative radiation, and
Have previously documented targetable EGFR mutations:
   NSCLC that harbors EGFR Exon 19 deletion
   NSCLC that harbors EGFR L858R mutation
   NSCLC with activating EGFR exon20 insertion
   NSCLC with exon 18/21 atypical mutations
   NOTE: EGFR deletion/mutation must have been previously documented by a certified test (either from tissue or ctDNA from blood is allowed)
Are anti-PD1/PD-L1 naïve, and
Have received a third generationTKI treatment
   For patients whose tumors harbor previously documented EFGR Exon19 deletion or L858R mutation, prior osimertinib or other third generation TKI treatment is required
Have received prior treatment with platinum-doublet chemotherapy
Small cell transformation is excluded

### Study Population

Dose Escalation: Study population will consist of patients with selected advanced solid tumors who have exhausted therapeutic options that are expected to provide meaningful clinical benefit, except for patients with malignancies where anti-PD-1/PD-L1 therapies have demonstrated clinical benefit.

Dose Expansion: Study population will consist of specified cohorts of patients with selected advanced solid tumors and are anti-PD-1/PD-L1 naïve.

### Inclusion Criteria

A patient must meet the following criteria to be eligible for inclusion in the study:
1. ≥18 years of age
2. Has an Eastern Cooperative Oncology Group (ECOG) performance status of 0 or 1
3. Has histologically or cytologically confirmed cancer that meets the following criteria:
   a. Dose Escalation:
   a. Has metastatic disease, or locally advanced disease that is not a candidate for curative surgery or curative radiation.
   b. Is not a candidate for an approved indication of an anti-PD-1 or PD-L1 therapy, or such therapy is otherwise not available to the patient (alone or in combination). Reason for ineligibility must be documented.
   c. Has exhausted all therapeutic options that are expected to provide meaningful clinical benefit, either through disease relapse, treatment refractory disease, or intolerance. Patients with malignancies where anti-PD-1/PD-L1 therapies have demonstrated clinical benefit are exempt from this requirement.
   d. Has any of the following cancer types:
      a. Colorectal cancer that is microsatellite stable as documented by local pathology
      b. Gastric or Gastroesophageal junction cancer
      c. Esophageal cancer
      d. Breast cancer (ductal or lobular carcinoma, regardless of receptor status)
      e. NSCLC (any PD-L1 expression)
      f. Head and neck squamous cell carcinoma (SCC)
      g. Nasopharyngeal carcinoma
      h. Cervical carcinoma
      i. Anal carcinoma
      j. Mesothelioma
      k. Prostate adenocarcinoma
      l. Renal cell carcinoma (chromophobe, clear cell, or papillary)
      m. Gallbladder/cholangiocarcinoma
      n. Urothelial carcinoma
      o. Pancreatic carcinoma
      p. Penile SCC
      q. Vulvovaginal carcinoma
      r. Additional non-CNS tumor types for which the investigator is able to demonstrate elevated EGFR expression in the tumor may be eligible after discussion with sponsor

NOTE: Small cell/large cell/neuroendocrine, and sarcomatoid histologies are excluded

b. Dose Expansion Cohorts:
a. Cohort A: Patients with metastatic TNBC who:
   a. Are not candidates for curative surgery or curative radiation
   b. Are not candidates for anti-PD-1 or anti-PD-L1 therapy in an approved indication, or such therapy is otherwise not available to the patient (eg, PD-L1 ICS not meeting score for indicated use, anti-PD-1 or anti-PD-L1 therapy is not accessible to the patient by reimbursement, contraindication to protein-bound paclitaxel, etc)
   c. Have previously documented triple negative cancer (ER-/PR-/Her2-), by local pathology
b. Cohort B: Patients with metastatic CSCC or locally advanced CSCC who are not candidates for curative surgery or curative radiation
c. Cohort C: Patients with histologically or cytologically documented locally advanced or metastatic NSCLC disease who:
   a. Are not candidates for curative surgery or curative radiation
   b. Do not have previously documented targetable molecular alterations (eg, ALK, ROS1, EGFR, Met Ex14, etc)
   c. Have received no prior systemic treatment for recurrent or metastatic NSCLC (adjuvant or neoadjuvant systemic treatments will not be counted as a prior line)
d. Cohort D: Patients with histologically or cytologically documented locally advanced or metastatic HNSCC disease who:
   a. Are not candidates for curative surgery or curative radiation
   b. Have previously documented PD-L1 expression of CPS ≥1% by IHC assay performed on a specimen collected within the past 3 months
   c. Have received no prior systemic treatment for recurrent or metastatic HNSCC (adjuvant or neoadjuvant systemic treatments will not be counted as a prior line)
e. Cohort E: Patients with metastatic CRC who have previously documented MSS disease, and who:
   a. Are not candidates for curative surgery or curative radiation
   b. May have active metastases are present in the liver and/or peritoneum at the time of screening
   NOTE: Ablated and resected disease is not considered active
   c. Are microsatellite stable per previously documented result by local pathology report from the patient's medical history
   d. Have received at least one line of therapy in the relapsed/metastatic setting
      Patients with previously documented RAS wild type disease must have received anti-EGFR therapy or have a documented reason why anti-EGFR therapy was not appropriate
      Patients must have received anti-VEGF therapy or have a documented reason why anti-VEGF therapy was not appropriate
f. Cohort F: Patients with metastatic CRC who have previously documented MSS disease, and who:
   a. Are not candidates for curative surgery or curative radiation
   b. No active metastases have been identified in the liver or peritoneum at the time of screening. NOTE: Ablated and resected disease is not considered active.
   c. Only sites of disease are present in lung(s) and/lung or lymph nodes
   d. Based on medical history, are microsatellite stable per previously documented result documented by local pathology report
   e. Have received at least one line of therapy in the relapsed/metastatic setting
      Patients with previously documented RAS wild type disease must have received anti-EGFR therapy or have a documented reason why anti-EGFR therapy was not appropriate.
      Patients must have received anti-VEGF therapy or have a documented reason why anti-VEGF therapy was not appropriate
g. Cohort G: Patients with previously documented histologically or cytologically documented locally advanced or metastatic EGFR mutated non-squamous NSCLC disease who:
   a. Are not candidates for curative surgery or curative radiation
   b. Have a previously documented targetable EGFR mutation:
   NSCLC that harbors EGFR Exon 19 deletion.
   NSCLC that harbors EGFR L858R mutation.
   NSCLC with activating EGFR exon20 insertion
   NSCLC with exon 18/21 atypical mutations
   NOTE: EGFR deletion/mutation must have been previously documented by a certified test (either from tissue or ctDNA from blood is allowed) from the patient's medical history.
b. Are chemotherapy naïve
c. Have received treatment with a third generation TKI
   a. For patients whose tumors harbor previously documented EFGR Exon19 deletion or L858R mutation, prior osimertinib or other third generation TKI treatment is required

### NOTES:

Stable CNS disease allowed. Stable is defined as no evidence of progression for at least 6 weeks on imaging obtained during the screening period; no evidence of new or enlarging brain metastases, and the patient does not require any immunosuppressive doses of systemic corticosteroids for management of brain metastases within 4 weeks of the first dose of study drug.
Small cell transformation is excluded
h. Cohort H: Patients with histologically or cytologically documented locally advanced or metastatic non-squamous NSCLC disease who:
   a. Are not candidates for curative surgery or curative radiation
   b. Have a previously documented targetable EGFR mutation:
      NSCLC that harbors EGFR Exon 19 deletion.
      NSCLC that harbors EGFR L858R mutation.
      NSCLC with activating EGFR exon20 insertion
      NSCLC with exon 18/21 atypical mutations
      NOTE: EGFR deletion/mutation must have been previously documented by any certified test (either from tissue or ctDNA from blood is allowed) from the patient's medical history.
   c. Have received treatment with a third generation TKI
      a. For patients whose tumors harbor previously documented EFGR Exon19 deletion or L858R mutation, prior osimertinib or other third generation TKI treatment is required
   d. Have received treatment with platinum-doublet chemotherapy

### Notes:

Stable CNS disease allowed. Stable is defined as no evidence of progression for at least 6 weeks on imaging obtained during the screening period; no evidence of new or enlarging brain metastases, and the patient does not require any immunosuppressive doses of systemic corticosteroids for management of brain metastases within 4 weeks of the first dose of study drug.
Small cell transformation is excluded
4. Expansion Cohorts Only: Is anti-PD-1/PD-L1 naïve, defined as never having previously been treated with a drug that targets the PD-1

Note: In Dose Escalation, prior anti-PD-1/PD-L1 therapy is allowed provided ≥1 month has passed before the first dose of study therapy AND the patient has recovered or returned to baseline from immune-mediated adverse events at least 1 month prior to initiation of the study drug. Endocrinopathies that are adequately managed with hormone replacement and vitiligo are not exclusionary. Patients who have permanently discontinued prior anti-PD-1/PD-L1 therapy due to drug related toxicity are not eligible.
5. Has at least 1 lesion that meets study criteria.
   Tumor lesions in a previously irradiated area are considered measurable if progression has been demonstrated (either by increase in size meeting RECIST 1.1 criteria for PD or biopsy proven viable tumor) in such lesions after radiation.

### For Patients in the CSCC Expansion Cohort Only:

a. There must be at least 1 measurable baseline lesion in which the longest diameter (LD) and the perpendicular diameter are both ≥10 mm if followed by digital medical photography. Non-measurable disease is defined as either unidimensionally measurable lesions, tumors with margins that are not clearly defined, or lesions with maximum perpendicular diameters less than 10 mm. Patients without measurable disease at baseline are not eligible for the study.
6. Willing to provide tumor tissue from newly obtained biopsy (at a minimum core biopsy) from a tumor site that has not been previously irradiated.

### Notes:

a. A previously irradiated lesion may be used if it is the only accessible lesion and it is documented that the lesion progressed after radiation.
b. If the patient has only a single RECIST 1.1 measurable and biopsy accessible lesion, a biopsy is recommended before baseline imaging to assure post-biopsy measurements are accurately taken.
c. This biopsy requirement may be waived on a limited basis after discussion between the investigator and sponsor, if the biopsy would pose a life-threatening risk to the patient in the opinion of the investigator (eg, if a retrocardiac mass is the only lesion). In these cases, archival tissue from a biopsy obtained within the last 6 months is required.
7. Has adequate organ and bone marrow function as documented by:
   a. Hemoglobin ≥8.0 g/dL
   b. Absolute neutrophil count (ANC) ≥1.0×10⁹/L
      Note: ANC of ≥0.5×10⁹/L will be permitted in patients of African descent who have a documented history of benign ethnic neutropenia
   c. Platelet count ≥75×10⁹/L
8. Serum creatinine ≤1.5×ULN or estimated glomerular filtration rate >30 mL/min (according to the investigator's institutionally accepted methodology, eg, Modification of Diet in Renal Response [MDRD], Chronic Kidney Disease Epidemiology Collaboration [CKD-EPI] equation). A 24-hour urine creatinine collection may substitute for the calculated creatinine clearance to meet eligibility criteria.
9. Grade <3 hypercalcemia or controlled on bisphosphonate therapy to grade <3
10. Hepatic function meeting the following criteria:
   a. Total bilirubin ≤1.5×upper limit of normal (ULN) (≤3×ULN if tumor liver involvement)
   b. Aspartate Aminotransferase (AST) ≤2.5×ULN (≤5×ULN if tumor liver involvement)
   c. Alanine Aminotransferase (ALT) ≤2.5×ULN (≤5×ULN if tumor liver involvement)
   d. Alkaline Phosphatase (ALP) ≤2.5×ULN (≤5×ULN if tumor liver or bone involvement)

### Notes:

Patients with tumor liver involvement and levels of AST ≥3×ULN or ALT ≥3×ULN, and bilirubin levels ≥2×ULN will be excluded regardless of the above criteria. Patients with Gilbert's syndrome do not need to meet total bilirubin requirements provided their total bilirubin is not greater than 150% of their historical level, and less than 2 mg/dL (34.2 μmol/L). Gilbert's syndrome must be documented appropriately as past medical history.
11. Does not need rapidly active therapy such that the 3-week REGN7075 monotherapy lead-in period before starting the combination of REGN7075 plus cemiplimab is not expected to be a clinically meaningful delay in receiving the cemiplimab component of the therapy. NOTE: This criterion does not apply to patients enrolling in cohorts with a concurrent start of REGN7075 and cemiplimab.
12. Has a life expectancy of at least 3 months.
13. Willing and able to comply with clinic visits and study-related procedures.
14. Able to provide informed consent signed by study participant or legally acceptable representative.

### Exclusion Criteria

A patient who meets any of the following criteria will be excluded from the study:
1. Is currently participating in another study of a therapeutic agent.
2. Has participated in any study of an investigational agent or an investigational device within 4 weeks of the first administration of study drug.
   a. Exception: Patients who have received or are enrolled in a study involving treatment with an investigational immunoPET (iPET) reagent may be enrolled in the study, as long as the iPET reagent does not target EGFR, CD28, PD-1, PD-L1, or PD-L2.
3. Has received treatment with an approved systemic therapy within 4 weeks of the first administration of study drug or has not yet recovered (ie, grade 1 or baseline) from any acute toxicit
4. Has received therapy with an anti-EGFR antibody therapy within the following drug specific window (approximately 5 half-lives):
5. Cetuximab: within 4 weeks of the first administration of REGN7075
6. Panitumumab: within 6 weeks of the first administration of REGN7075
7. Necitumumab: within 10 weeks of the first administration of REGN7075
8. Any other investigative agent that blocks EGFR via interaction with extracellular binding domain: within 5 half-lives of the first administration of REGN7075, after sponsor approval.
   a. NOTE: Kinase inhibitors that target EGFR will be permitted within the guidelines of exclusion criteria 3.
9. Has received radiation therapy or major surgery within 14 days of the first administration of study drug or has not recovered (ie, grade 1 or baseline) from adverse events.
10. Has received any previous systemic, non-immunomodulatory biologic therapy within 4 weeks of first administration of study drug.
11. Has had prior anti-cancer immunotherapy within 5 half-lives prior to study drug. Examples of immune modulating agents include blockers of CTLA-4, 4-1BB (CD137), or OX-40, therapeutic vaccines, PI3K-delta inhibitors, or cytokine anticancer treatments.
   a. Prior CAR-T therapy will be allowed regardless of cell half-life/persistence.
12. Has not recovered (ie, baseline) from immune-mediated adverse events prior to initiation of study drug. Endocrinopathies adequately managed with hormone replacement are not exclusionary.
13. Has a contraindication to receiving an anti-PD-1 therapy
14. Has known (previously documented) microsatellite instability high (MSI-high) or mismatch repair deficient cancer.
15. Has second malignancy that is progressing or requires active treatment, except:
   a. Non-melanoma skin cancer that has undergone potentially curative therapy
   b. Any tumor that has been deemed to be effectively treated with definitive local control
16. Has any condition requiring ongoing/continuous corticosteroid therapy (>10 mg prednisone/day or anti-inflammatory equivalent) within 1-2 weeks prior to the first dose of study drug. Physiologic replacement doses are allowed even if they are >10 mg of prednisone/day or equivalent, as long as they are not being administered for immunosuppressive intent. Inhaled or topical steroids are permitted, provided that they are not for treatment of an autoimmune or skin disorder.
   a. Note: Patients who require a brief course of steroids (up to 2 days in the week before enrollment), a brief course of corticosteroids for prophylaxis (eg, contrast dye allergy) or for treatment of non-autoimmune conditions (eg, delayed-type hypersensitivity reaction caused by contact allergen), or physiologic replacement, may be enrolled into the study.
17. Has ongoing or recent (within 5 years) evidence of significant autoimmune disease or any other condition that required treatment with systemic immunosuppressive treatments. The following are not exclusionary: vitiligo, childhood asthma that has resolved, endocrinopathies (such as hypothyroidism or type 1 diabetes) that require only hormone replacement.
18. Has untreated or active primary brain tumor, CNS metastases, leptomeningeal disease, or spinal cord compression.
   a. NOTE: Patients with previously treated central nervous system metastases or spinal cord compression are not excluded if:
   b. No evidence of progression for at least 4 weeks prior to the first dose of study drug, and any neurologic symptoms have returned to baseline, and
   c. No evidence of new or enlarging central nervous system metastases, and
   d. No requirement for systemic corticosteroids for management of central nervous system metastases or spinal cord compression within 4 weeks prior to first dose of study drug
19. Has encephalitis, meningitis, organic brain disease (eg, Parkinson's disease) or uncontrolled seizures within 1 year prior to the first dose of study drug
20. Has a marked baseline prolongation of QT/QTc interval or risk factors for prolonged QTc such as:
   a. Repeated demonstration of a QTc interval >470 msec at baseline
   b. A history of additional risk factors for torsade de pointes (TdP), or family history of Long QT Syndrome
21. A history of myocardial infarction, cardiac event, congestive heart failure (NYHA Class Il or higher), or cardiac arrythmia, in the last 12 months.
   a. NOTE: A history of arrythmia that was transient and did not require any medical intervention will be permitted provided the episode was not in the last 6 months prior to enrollment. Premature Atrial Contractions (PACs) or Premature Ventricular Contractions (PVCs) not meeting criteria for a specific arrythmia are permitted regardless.
22. Any patient with active inflammatory skin disease (eg, psoriasis, eczema, etc.), or inflammatory skin disease (regardless of activity) that requires ongoing medication (topical or systemic), or a history of inflammatory skin disease within the last 5 years.
23. Any of the following skin findings ongoing at baseline, regardless of grade:
   a. Bullous dermatitis
   b. Dermatitis exfoliative
   c. Erythroderma
   d. Erythema multiforme
   e. Rash acneiform
   f. Rash generalized
   g. Rash maculo-papular
   h. Other skin toxicities (other than grade ≤1), not specified, with any inflammatory or destructive characteristics such as infiltration, erythema, ulceration, blistering, exfoliation, etc.
   i. NOTE: Skin findings that are grade ≤1 that are anticipated to be transient will not be exclusionary. Additionally, grade ≤1 hypersensitivity reactions that are anticipated to be transient are not exclusionary.
24. Has a known history of, or any evidence of interstitial lung disease, or active, non-infectious pneumonitis within 5 years prior to the first dose of study drug. A history of radiation pneumonitis in the radiation field is permitted as long as pneumonitis resolved ≥6 months prior to first dose of study therapy.
25. Has any indwelling drains or stents (eg, nephrostomy tube, biliary stent, etc) that are intended to preserve organ function or facilitate drainage of an occlusion.
   a. NOTE
   b. Urinary catheterization of bladder or neobladder will not be exclusionary
   c. Ports or indwelling intravenous lines are permitted
   d. Coronary artery stents are not exclusionary, provided patients meet criteria #17
26. Has uncontrolled infection with human immunodeficiency virus, hepatitis B, or hepatitis C infection; or diagnosis of immunodeficiency.
   a. NOTES:
   b. Patients will be tested for hepatitis C virus (HCV) and hepatitis B virus (HBV) at screening.
   c. Patients with known HIV infection who have controlled infection (undetectable viral load (HIV RNA PCR) and CD4 count above 350 either spontaneously or on a stable antiviral regimen) are permitted. For patients with controlled HIV infection, monitoring will be performed per local standards.
   d. Patients with hepatitis B (HepBsAg+) who have controlled infection (serum hepatitis B virus DNA PCR that is below the limit of detection AND receiving anti-viral therapy for hepatitis B) are permitted. Patients with controlled infections must undergo periodic monitoring of HBV DNA. Patients must remain on anti-viral therapy for at least 6 months beyond the last dose of investigational study drug.
   e. Patients who are hepatitis C virus antibody positive (HCV Ab+) who have controlled infection (undetectable HCV RNA by PCR either spontaneously or in response to a successful prior course of anti-HCV therapy) may be enrolled into the study
27. Has required treatment with anti-infectives within 4 weeks of first dose of study drug, or has any ongoing infection (except as otherwise specified in exclusion criterion).
28. Has received a live vaccine within 4 weeks of planned start of study drug.
29. Has had prior allogeneic stem cell transplantation, autologous stem cell transplantation, or solid organ transplant at any time.
30. Has known allergy or hypersensitivity to cemiplimab or components of study drug.
31. Has known psychiatric or substance abuse disorders that would interfere with participation with the requirements of the study.
32. Has any medical condition, co-morbidity, physical examination finding, or metabolic dysfunction, or clinical laboratory abnormality that renders the patient unsuitable for participation in a clinical study due to high safety risks and/or potential to affect interpretation of results of the study
33. Members of the clinical site study team and/or his/her immediate family, unless prior approval granted.
34. Patients with a positive serum hCG pregnancy test must have pregnancy medically ruled out to be eligible for study. Breastfeeding women are also excluded.
35. Continued sexual activity in women of childbearing potential (WOCBP)*, or sexually active men, who are unwilling to practice highly effective contraception prior to the initial dose/start of the first treatment, during the study, and for at least 6 months after the last dose. Highly effective contraceptive measures include:
   a. stable use of combined (estrogen and progestogen containing) hormonal contraception (oral, intravaginal, transdermal) or progestogen-only hormonal contraception (oral, injectable, implantable) associated with inhibition of ovulation initiated 2 or more menstrual cycles prior to screening
   b. intrauterine device (IUD); intrauterine hormone releasing system (IUS);
   c. bilateral tubal ligation or occlusion
   d. vasectomized partner (provided that the male vasectomized partner is the sole sexual partner of the WOCBP study participant and that the vasectomized partner has obtained medical assessment of surgical success for the procedure)
   e. and/or sexual abstinence†,‡.
36. WOCBP are defined as women who are fertile following menarche until becoming postmenopausal, unless permanently sterile. Permanent sterilization methods include hysterectomy, bilateral salpingectomy, and bilateral oophorectomy.
   a. A postmenopausal state is defined as no menses for 12 months without an alternative medical cause. A high follicle stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. The above definitions are according to Clinical Trial Facilitation Group (CTFG) guidance. Pregnancy testing and contraception are not required for women who are post-menopausal or permanently sterile.† Sexual abstinence is considered a highly effective method only if defined as refraining from heterosexual intercourse during the entire period of risk associated with the study drugs. The reliability of sexual abstinence needs to be evaluated in relation to the duration of the clinical trial and the preferred and usual lifestyle of the patient.
   b. ‡ Periodic abstinence (calendar, symptothermal, post-ovulation methods), withdrawal (coitus interruptus), spermicides only, and lactational amenorrhoea method (LAM) are not acceptable methods of contraception. Female condom and male condom should not be used together.
37. For Cohorts G and H only: Small cell transformations.

### Study Treatments

REGN7075: 0.1 mg up to 900 mg will be administered every week (QW), or 0.1 mg up to 2700 mg will be administered QW or every 3 weeks (Q3W), by IV infusion or by subcutaneous (SC) injection at target dose levels. For doses 100 mg and above, REGN7075 will be administered using a step-up with or without split dosing schedule.

Cemiplimab: 350 mg will be administered concomitantly Q3W by IV infusion over 30 minutes or by SC injection.

When both drugs are administered on the same day, REGN7075 will be administered prior to cemiplimab.

Platinum-based doublet chemotherapy administered IV Q3W for 4 cycles (followed by pemetrexed maintenance for those patients initially assigned to receive a pemetrexed-containing regimen).

The choice of chemotherapy will be one of the regimens shown in Table 11.

TABLE 11
Guidelines for Platinum-based Chemotherapy regimens

| | | | Maintenance |
|---|---|---|---|
| Option | Chemotherapy Regimen | Dosing Frequency | Therapy |
| 1 | Paclitaxel 200 mg/m² IV | Day 1 every 21 days | No maintenance |
| | plus carboplatin AUC of | (Q3W) for 4 cycles. | therapy |
| | 5 or 6 mg/mL/minute IV | Calculate dose of | |
| | | carboplatin using the | |
| | | Calvert formula. | |
| 2 | Paclitaxel 200 mg/m² IV | Day 1 every | No maintenance |
| | plus cisplatin 75 mg/m² | 21 days (Q3W) for 4 | therapy |
| | IV | cycles | |
| 3 | Pemetrexed 500 mg/m² IV | Day 1 every | Mandatory |
| | plus carboplatin AUC of | 21 days (Q3W) for 4 | pemetrexed |
| | 5 or 6 mg/mL/minute IV | cycles. | maintenance |
| | | Calculate dose of | 500 mg/m² IV |
| | | carboplatin using the | day 1 every 21 |
| | | Calvert formula. | days; pemetrexed |
| | | | maintenance |
| | | | according to local |
| | | | prescribing information |
| | | | and practice |
| | | | guidelines. |
| 4 | Pemetrexed 500 mg/m² IV | Day 1 every 21 days | Mandatory |
| | plus cisplatin 75 mg/m² | (Q3W) for 4 cycles | pemetrexed |
| | IV | | maintenance |
| | | | 500 mg/m² IV day 1 |
| | | | every 21 days; |
| | | | pemetrexed |
| | | | maintenance |
| | | | according to local |
| | | | prescribing |
| | | | information |
| | | | and practice |
| | | | guidelines. |

Abbreviations:
AUC = area under the curve;
IV = intravenous;
N/A = not applicable,
Q3W = every 3 weeks

Chemotherapy will be administered after completion of cemiplimab administration. When chemotherapy is administered the same day as REGN7075 and cemiplimab, the whole dose of REGN7075 will be administered first, followed by cemiplimab, and lastly chemotherapy.

### Study Endpoints

Primary endpoints for treatments are determined based on the following assessments.

### Dose Escalation:

The incidence of dose-limiting toxicities (DLTs) during the DLT period
The incidence and severity of treatment emergent adverse events (TEAEs)/adverse events of special interest (AESIs)/serious adverse events (SAEs) and grade ≥3 laboratory abnormalities

### Dose Expansion:

Objective Response Rate (ORR) by Response Evaluation Criteria in Solid Tumors version 1.1 (RECIST 1.1) and/or composite response criteria (depending on the subject's baseline evaluation criteria).

Secondary endpoints for treatments are determined based on the following assessments:

### Dose Escalation

Drug concentrations of REGN7075 in serum, and drug concentrations of cemiplimab in serum
Objective response rate (ORR), progression free survival (PFS), duration of response (DOR), complete response (CR) rate, and disease control rate (DCR) rate by Response Evaluation Criteria in Solid Tumors version 1.1 (RECIST 1.1) and/or composite response criteria (depending on the subject's baseline evaluation criteria)
Overall survival (OS)
Incidence of anti-drug antibodies (ADA) to REGN7075 and cemiplimab

### Dose Expansion

The incidence and severity of TEAEs/AESIs/SAEs and grade ≥3 laboratory abnormalities.
Drug concentrations of REGN7075 in serum, and drug concentrations of cemiplimab in serum
Incidence of ADA to REGN7075 and cemiplimab
PFS, DOR, DCR, and CR rate by RECIST 1.1 and/or composite response criteria (depending on the subject's baseline evaluation criteria)
OS
Patient reported QoL, symptoms, functioning and general health status (per EORTC

QLQ-C30, EORTC QLQ-CR29 in CRC patients, EORTC QLQ-BR23 in breast cancer patients, EORTC QLQ-LC13 in NSCLC patients, EORTC QLQ-HN35 in HNSCC patients, and EQ-5D-5L).

### Procedures and Assessments

Treatments will be assessed using the following procedures and assessments:
Only at Screening/Baseline: Height, brain imaging, coagulation assessment, and evaluation of archival or fresh tumor.
Efficacy: Radiographic disease assessment (computed tomography [CT], magnetic resonance imaging [MRI], and/or digital photography). For subjects with disease that can be measured radiologically, disease will be radiologically evaluated according to RECIST 1.1. For subjects whose CSCC lesions are evaluable on the skin, composite response criteria should be used in combination with radiologic imaging if appropriate.
Safety: Vital signs (including temperature, sitting blood pressure, pulse, and respiration), physical examination, Eastern Cooperative Oncology Group (ECOG) performance status, weight, electrocardiogram, adverse events (AEs), hematology, blood chemistry, c-reactive protein (CRP), thyroid stimulating hormone (TSH), pregnancy test, urinalysis
Pharmacokinetics and immunogenicity sampling
Biomarkers and exploratory research: serum cytokines, exploratory tumor biopsy (or archival tissue), whole blood for immune monitoring, plasma for ctDNA, peripheral blood mononuclear cells (PBMC) for immunophenotyping, whole blood for DNA/RNA for T-cell repertoire analysis, serum and plasma for exploratory biomarkers.

### Results

Preliminary efficacy and safety results are described in the Examples below. Results to-date include partial and complete responses in many cohorts.

### Example 3

### Initial Dose-Escalation Results of a Phase 1/2 Study of REGN7075 in Combination with Cemiplimab (anti-PD-1) in Subjects with Advanced Solid Tumors

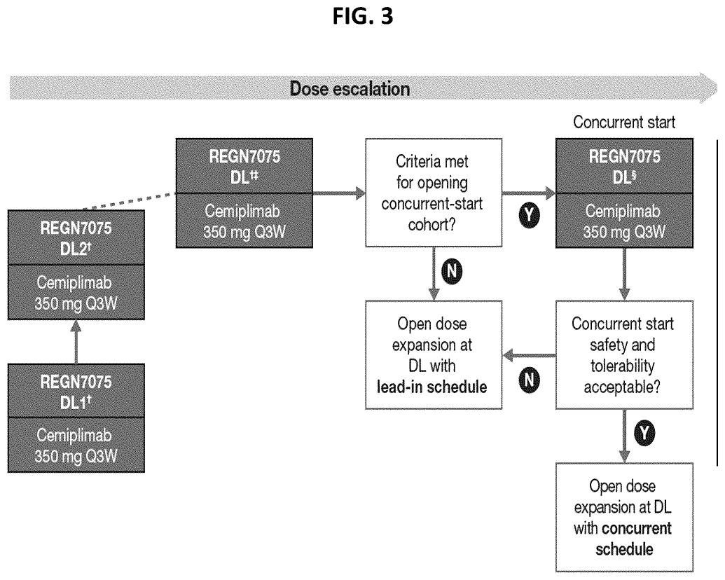

A first-in-human, open-label, phase 1/2 dose-escalation and expansion study was conducted to evaluate the safety, tolerability, pharmacokinetics, and preliminary antitumor activity of REGN7075 (EGFRxCD28) in combination with cemiplimab (anti-PD-1) in subjects with advanced solid tumors (FIG. 3). The study protocol is described in detail in Example 2.

In the dose-escalation phase (Bayesian optimal interval design; Part 1), heavily pre-treated subjects with advanced solid tumors received a lead-in of REGN7075 monotherapy every week for 3 weeks, followed by combination therapy with cemiplimab 350 mg every 3 weeks. Planned dose levels (DL) of REGN7075 were 0.03, 0.1, 0.3, 1, 3, 10, 30, 100, 300, and 900 mg. Primary objective was to assess safety and tolerability of REGN7075 in combination with cemiplimab.

As of the data cutoff date, 18 subjects (median age, 53.5 years, 56% female) were treated in the dose-escalation phase (Table 12), up to the 30 mg DL for REGN7075 in combination with cemiplimab. Most subjects (67%) were treated for microsatellite stable colorectal cancer. No subjects experienced dose-limiting toxicities; maximum tolerated dose was not reached.

Table 13 summarizes treatment-emergent adverse events (TEAEs) and treatment-related adverse events (TRAEs). The most frequent TEAEs (any grade) were increases in aspartate aminotransferase (AST), constipation, and fatigue (33% [n=6] each). The most frequent TRAEs (any grade) were fatigue (17% [n=3]), increases in AST, diarrhea, hypothyroidism, pyrexia, and rash (11% [n=2] each). One subject developed cytokine release syndrome which was characterized by isolated grade 1 fever without hypotension nor hypoxia. Five recorded deaths were not during study treatment and not attributed to study drug(s).

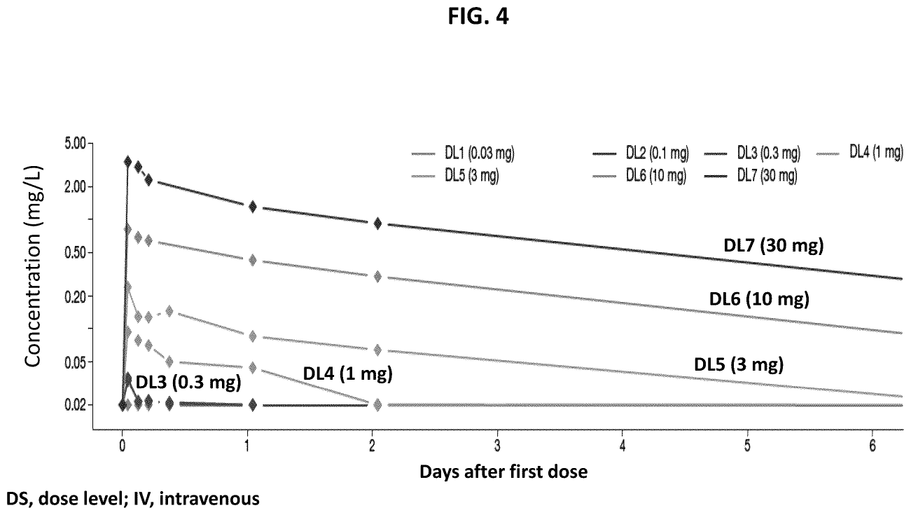

Serum concentration of REGN7075 following intravenous administration of the first dose of REGN7075 was measured. Maximum concentration (Cmax) and area under the curve (AUC) values were greater than dose proportional at the 30 mg dose, whereas minimum concentration (Cmin) values were dose proportional at all doses (FIG. 4). Ongoing PK evaluation suggests possible target-mediated effects.

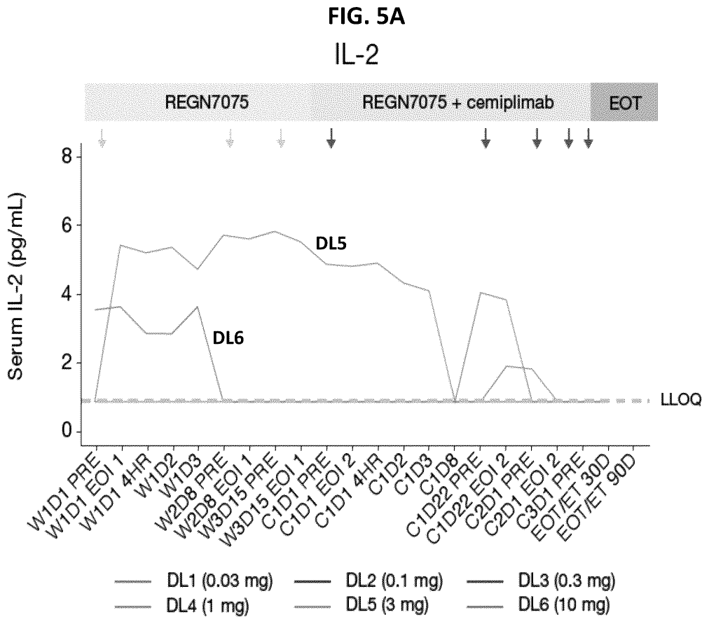

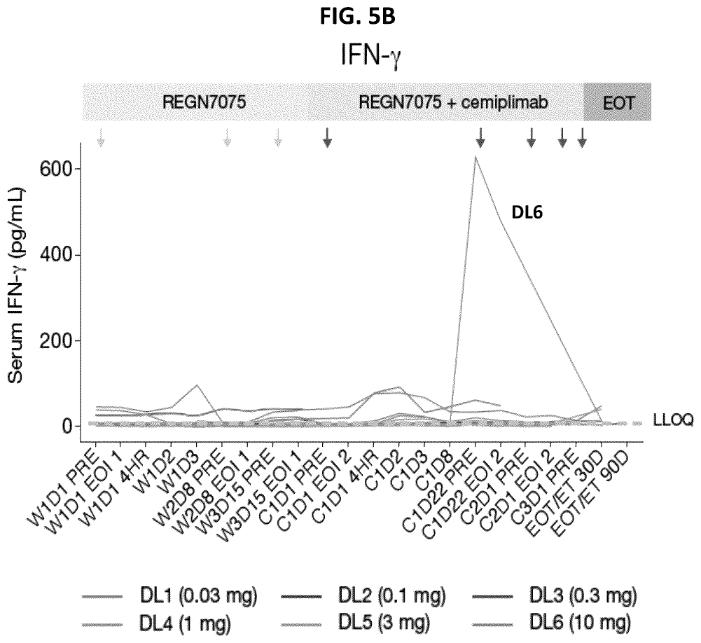

T cell activation-associated cytokines were detected in monotherapy lead-in and combination dosing. IL-2 was induced in one patient following the first dose of REGN7075 (1 mg) (FIG. 5A). IFN-γ0 was induced in multiple patients after they received REGN7075 alone or in combination with cemiplimab (FIG. 5B).

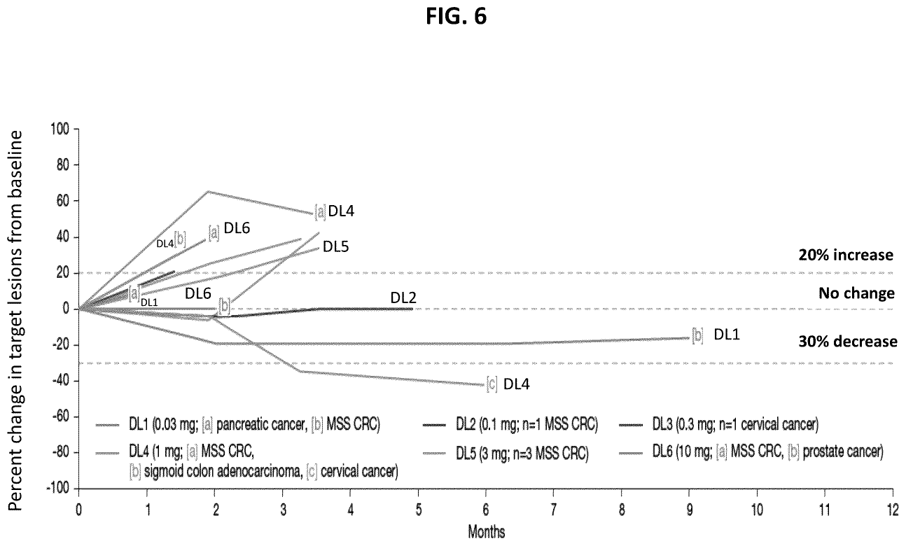

Treatment and evaluation of the dose-escalation phase are on going. Nevertheless, preliminary data shows that of all 18 subjects treated in dose escalation, 1 PD-1 negative subject who received 1 mg REGN7075 with cemiplimab for cervical cancer surprisingly achieved an ongoing partial response (DL4 [c], FIG. 6).

In this dose-escalation study, REGN7075 was safely administered up to the 30 mg dose level in combination with cemiplimab without dose-limiting toxicities. Early data indicates that novel agent REGN7075 was generally well tolerated, with preliminary anti-tumor activity. There were no signs of the broad-scale immune activation or cytokine release syndrome observed with the CD28 superagonist TGN1412.

TABLE 12
Demographics and baseline characteristics

| | Total | (N = 18) |
|---|---|---|
| Age (years) | 53.5 | (47-65) |
| Median (Q1-Q3) | | |
| Sex, n (%) | | |
| Male | 8 | (44%) |
| Female | 10 | (56%) |
| ECOG PS, n (%) | | |
| 0 | 8 | (44%) |
| 1 | 10 | (56%) |
| Cancer diagnosis, n (%) | | |
| Colorectal cancer, microsatellite stable | 12 | (67%) |
| Pancreatic carcinoma | 3 | (17%) |
| Cervical carcinoma | 2 | (11%) |
| Prostate adenocarcinoma | 1 | (6%) |
| Membrane cytoplasmic H score, median (Q1-Q3) | 115.0 | (0-190) |
| PD-L1 expression level, n (%)† | | |
| 0% | 3 | (17) |
| <1% | 3 | (17) |
| 1% | 1 | (6) |
| Unknown | 11 | (61) |

†Percentage of tumor cells with PD-L1 staining present in tumor cell membranes at any intensity above background.
Data are collected in whole integers except for <1.
ECOG PS, Easter Cooperative Oncology Group performance status
PD-L1, programmed cell death-ligand 1;
Q, quartile.

TABLE 13
Summary of treatment-emergent adverse events (TEAE)
and treatment-related adverse events (TRAE)

| | Total (N = 18) | |
|---|---|---|
| | All grades | Grades 3-5 |
| TEAE | | |
| Number of subjects with any TEAE, n (%) | 17 (94%) | 7 (39%) |
| Most common TEAEs (in >10% of subjects) | | |
| Aspartate aminotransferase increased | 6 (33%) | 0 |
| Constipation | 6 (33%) | 1 (6%) |
| Fatigue | 6 (33%) | 0 |
| Diarrhea | 5 (28%) | 0 |
| Vomiting | 5 (28%) | 0 |
| Blood bilirubin increased | 4 (22%) | 1 (6%) |
| Nausea | 4 (22%) | 0 |
| Abdominal distension | 3 (17%) | 0 |
| Abdominal pain | 3 (17%) | 0 |
| Alanine aminotransferase increased | 3 (17%) | 0 |
| Arthralgia | 3 (17%) | 0 |
| Cough | 3 (17%) | 0 |
| Hypomagnesemia | 3 (17%) | 0 |
| Anemia | 2 (11%) | 0 |
| Back pain | 2 (11%) | 0 |
| Blood alkaline phosphatase increased | 2 (11%) | 1 (6%) |
| Blood creatinine increased | 2 (11%) | 0 |
| Decreased appetite | 2 (11%) | 0 |
| Dehydration | 2 (11%) | 0 |
| Dry skin | 2 (11%) | 0 |
| Hypothyroidism | 2 (11%) | 0 |
| Nasal congestion | 2 (11%) | 0 |
| Pruritus | 2 (11%) | 0 |
| Pyrexia | 2 (11%) | 0 |
| Rash maculo-papular | 2 (11%) | 0 |
| TRAE | | |
| Number of subjects with any TRAE for either | 14 (78%) | 0 |
| REGN7075 (including lead-in period) and/or | | |
| with cemiplimab, n (%) | | |
| Most common TRAEs (in >10% of subjects) | | |
| Fatigue | 3 (17%) | 0 |
| Aspartate aminotransferase increase | 2 (11%) | 0 |
| Diarrhea | 2 (11%) | 0 |
| Hypothyroidism | 2 (11%) | 0 |
| Pyrexia | 2 (11%) | 0 |
| Rash maculo-papular | 2 (11%) | 0 |

### Example 4

### Updated Results for the Clinical Trial of REGN7075 (EGFRxCD28) Alone and in Combination with an Anti-PD-1 Antibody, e.g., Cemiplimab

An open-label, Phase 1/2, first-in-human study evaluates the safety, tolerability, pharmacokinetics, and preliminary anti-tumor activity of REGN7075 (EGFRxCD28) alone and in combination with cemiplimab (anti- programmed cell death [PD]-1) in subjects with advanced solid tumors was conducted (see Example 2). Subjects must have a protocol-defined advanced solid tumor, have an Eastern Cooperative Oncology Group performance status of 0 or 1, and be naive to anti-PD-1/anti-PD-ligand(L)1 therapy.

This study included a dose-escalation (Bayesian optimal interval design; Part 1) and a dose-expansion phase (Part 2). In Part 1, heavily pre-treated subjects with advanced solid tumors received a lead-in of REGN7075 monotherapy every week for 3 weeks followed by combination therapy with cemiplimab 350 mg every 3 weeks. Planned dose levels (DLs) of REGN7075 were 0.03, 0.1, 0.3, 1, 3, 10, 30, 100, 300, and 900 mg.

Once the recommended Phase 2 dose is determined in Part 1, five tumor-specific expansion cohorts will be opened in Part 2: colorectal cancer (microsatellite stable [MSS]), non-small cell lung cancer (NSCLC, PD-L1 ≥50%), triple-negative breast cancer, cutaneous squamous cell carcinoma, and head and neck squamous cell carcinoma (PD-L1 selected, combined positive score ≥1). Subjects with MSS-CRC with RAS or BRAF wild type mutations must have received anti-EGFR therapy or anti-vascular endothelial growth factor (VEGF therapy). Primary endpoints are safety and tolerability of REGN7075 alone or in combination with cemiplimab for Part 1, and objective response rate per Response Evaluation Criteria in Solid Tumors version 1.1 for Part 2. For Part 2, a secondary objective is to assess the effect of REGN7075 on subject-reported outcomes, including health related quality of life, as measured by several validated instruments including the European Organisation for Research and Treatment of Cancer Quality of Life Questionnaire (EORTC QLQ-C30), and EORTC QLQ-CR29 (CRC subjects only).

As of Sep. 13, 2022, 30 subjects were treated in the dose-escalation phase, up to the 300 mg DL for REGN7075 in combination with cemiplimab.

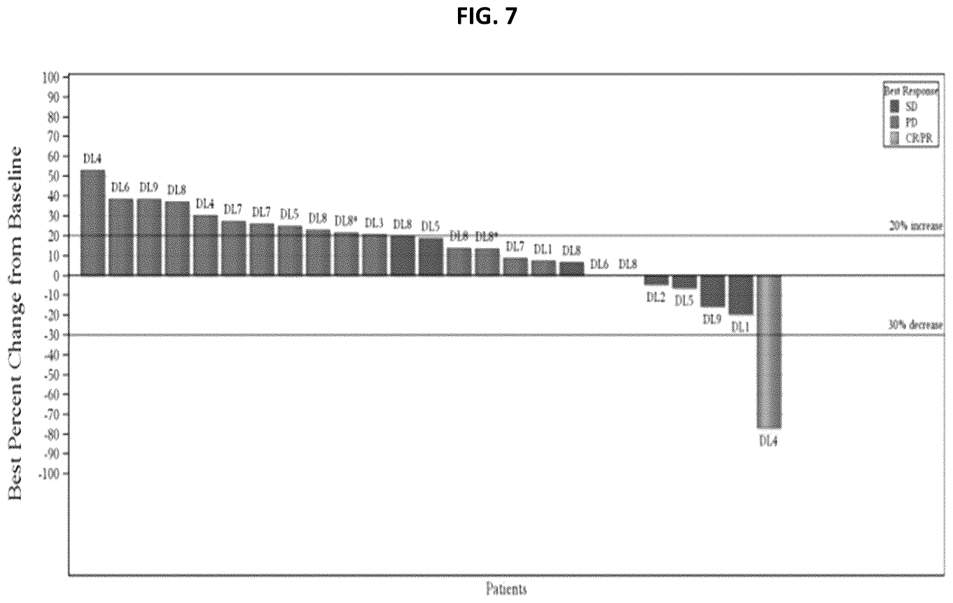

Updated results, as of December 2022, show preliminary partial responses in two patients with MSS-CRC (one each from DL1 and DL9) and complete response in the cervical cancer patient in DL4 (FIG. 7).

### Example 5

### Updated Clinical Results

This Example describes updated results for the open-label, Phase 1/2, first-in-human, global study to evaluate the safety, tolerability, PK and anti-tumor activity of REGN7075 ±cemiplimab in patients with advanced solid tumors (NCT04626635) (see Example 2).

The study includes a dose-escalation (Part 1) and a dose-expansion phase (Part 2). Patients must have an advanced solid tumor type likely to express EGFR, ECOG performance status of 0/1 and be anti-PD(L)-1 therapy-naïve (Part 2 only). In Part 1, patients will receive a 3-week REGN7075 monotherapy QW lead-in (except for cohorts C and G) prior to combination therapy with REGN7075 QW or Q3W+cemiplimab Q3W. Concurrent Q3W dosing with cemiplimab may be conducted for dose levels for which the lead-in is tolerable. After a tolerable dose and schedule of drug administration is identified in Part 1, Part 2 may include 8 tumor-specific expansion cohorts: triple-negative breast cancer (A); cutaneous squamous cell carcinoma (B); non-small cell lung cancer (NSCLC; C); head and neck squamous cell carcinoma (D); microsatellite stable colorectal cancer (MSS-CRC) with active liver and/or peritoneal metastases (E); MSS-CRC with lung/lymph node metastases (F); EGFR-mutant NSCLC post-third generation TKI (G); EGFR-mutant NSCLC post-third generation TKI and post-platinum-doublet chemotherapy (H). Cohorts C and G will also receive 4 cycles of platinum-based chemotherapy concurrently with cemiplimab. Primary endpoints: Part 1, safety and tolerability of REGN7075±cemiplimab; Part 2, ORR (REGN7075+cemiplimab±chemotherapy; RECIST 1.1). Part 1 and 2 secondary objectives include: OS, PFS, DoR, CR rate and DCR; immunogenicity of REGN7075 and cemiplimab; and characterisation of PK.

The study is expected to enroll ˜769 patients: ˜221 patients in Part 1 and ˜548 patients in Part 2.

As of 19 Jul. 2023, REGN7075 has been shown to be well tolerated in over 60 patients with advanced solid tumors who were treated with up to 900 mg of REGN7075 administered IV alone, and in the combination with cemiplimab. No patient experienced treatment-related adverse events leading to death. No patient experienced treatment-related AEs leading to permanent study drug discontinuation. No patient had a protocol-defined dose limiting toxicity within the dose limiting toxicity period. Events of IRRs were mostly grade 2 or grade 1 and symptoms of IRR remained clinically manageable.

As of 5 Sep. 2023, 93 patients were enrolled in the Part 1. Several partial and complete responses have been reported in patients in the study. This study is ongoing and open to enrolment.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

1. A method for treating a cancer in a subject, the method comprising administering to the subject a therapeutically effective amount of a bispecific antibody or antigen-binding fragment thereof comprising a first antigen-binding domain that binds cluster of differentiation factor 28 (CD28) and a second antigen-binding domain that binds epidermal growth factor receptor (EGFR) in combination with an antibody or antigen-binding fragment thereof that specifically binds programmed death receptor-1 (PD-1), thereby treating the cancer in the subject.

2. The method of claim 1, wherein the cancer is a solid tumor.

3. The method of claim 1, wherein the cancer is a EGFR-expressing cancer.

4. The method of claim 1, wherein the cancer is selected from esophageal carcinoma, lung squamous cell carcinoma, lung adenocarcinoma, cervical cancer, endometrial adenocarcinoma, bladder cancer, urothelial carcinoma, lung cancer, non-small cell lung cancer, colorectal cancer, sigmoid colon adenocarcinoma, rectal cancer, endometrial cancer, skin cancer, head & neck squamous cell carcinoma, brain cancer, glioblastoma multiforme, non-CNS tumor, cutaneous squamous cell carcinoma, breast cancer, gastric cancer, gastroesophageal cancer, gastroesophageal adenocarcinoma, pancreatic cancer, prostate cancer, ovarian cancer, melanoma, nasopharyngeal carcinoma, anal carcinoma, mesothelioma, renal cell carcinoma, gallbladder/cholangiocarcinoma, pancreatic carcinoma, penile squamous cell carcinoma, or vulvovaginal carcinoma.

5. The method of claim 1, further comprising selecting a subject, wherein the subject has an advanced solid tumor.

6. The method of claim 1, wherein the subject has at least one of the following criteria, or is selected on the basis of at least one of the following criteria:
a. Has metastatic disease, or locally advanced disease that is not a candidate for curative surgery or curative radiation;
b. Is not a candidate for an approved indication of an anti-PD-1 or PD-L1 therapy, or such therapy is otherwise not available to the subject (alone or in combination);
c. Has exhausted all therapeutic options that are expected to provide meaningful clinical benefit, either through disease relapse, treatment refractory disease, or intolerance except subjects with malignancies where anti-PD-1/PD-L1 therapies have demonstrated clinical benefit; and/or
d. Has any of the following cancer types:
(a) Colorectal cancer that is microsatellite stable as documented by local pathology; (b) Gastric or Gastroesophageal junction cancer; (c) Esophageal cancer; (d) Breast cancer (ductal or lobular carcinoma, regardless of receptor status); (e) NSCLC (any PD-L1 expression); (f) Head and neck squamous cell carcinoma (SCC); (g) Nasopharyngeal carcinoma; (h) Cervical carcinoma; (i) Anal carcinoma; (j) Mesothelioma; (k) Prostate adenocarcinoma; (1) Renal cell carcinoma (chromophobe, clear cell, or papillary); (m) Gallbladder/cholangiocarcinoma; (n) Urothelial carcinoma; (o) Pancreatic carcinoma; (p) Penile SCC; (q) Vulvovaginal carcinoma; or (r) Additional non-CNS tumor types for which elevated EGFR expression in the tumor is demonstrated.

7. The method of claim 1, wherein the subject has been treated with a prior therapy selected from radiation, surgery, chemotherapy, a PD-1 inhibitor, a PD-L1 inhibitor, an anti-VEGF therapy, CAR-T therapy, and/or an anti-EGFR therapy.

8. The method of claim 1, wherein the subject has not received prior anti-PD-1 therapy or anti-PD-L1 therapy.

9. The method of claim 1, wherein the subject has microsatellite-stable colorectal cancer (MSS CRC).

10. The method of claim 9, wherein the subject with microsatellite-stable colorectal cancer has, or is selected on the basis of, at least one of the following attributes: (a) has metastatic CRC; (b) is not a candidate for curative surgery or curative radiation; (c) may have active metastases are present in the liver and/or peritoneum at the time of screening; (d) no active metastases have been identified in the liver or peritoneum at the time of screening and the only sites of disease are present in lung(s) and/lung or lymph nodes; (e) has microsatellite stable as documented by pathology report; (f) has received at least one line of therapy in the relapsed/metastatic setting, wherein the therapy comprises anti-EGFR therapy or anti-VEGF therapy; or (g) is anti-PD-1/PD-L1 naïve, defined as never having previously been treated with a drug that targets PD-1.

11. The method of claim 1, wherein the subject has triple negative breast cancer (TNBC).

12. The method of claim 11, wherein the subject with TNBC has, or is selected on the basis of, at least one of the following attributes: (a) has metastatic TNBC; (b) is not a candidate for curative surgery or curative radiation; (c) is not a candidate for anti-PD-1 or anti-PD-L1 therapy in an approved indication, or such therapy is otherwise not available to the subject; (d) has triple negative cancer (ER-/PR-/Her2-), as documented by pathology report; or (e) is anti-PD-1/PD-L1 naïve, defined as never having previously been treated with a drug that targets PD-1.

13. The method of claim 1, wherein the subject has cutaneous squamous cell carcinoma (CSCC), wherein: (i) the subject is not a candidate for curative surgery or curative radiation; or (ii) the subject is anti-PD-1/PD-L1 naïve, defined as never having previously been treated with a drug that targets PD-1.

14. The method of claim 1, wherein the subject has non-small cell lung cancer (NSCLC).

15. The method of claim 14, wherein the subject has, or is selected on the basis of, at least one of the following attributes: (a) the subject has previously documented histologically or cytologically documented locally advanced or metastatic EGFR mutated non-squamous NSCLC disease; (b) has advanced or metastatic NSCLC; (c) is not a candidate for curative surgery or curative radiation; (d) has a previously documented targetable EGFR mutation (EGFR Exon 19 deletion, EGFR L858R mutation, EGFR exon20 insertion, or exon 18/21 atypical mutations); (e) is chemotherapy naïve; (f) has received treatment with platinum-doublet chemotherapy; (e) has received treatment with a third generation TKI; or (g) is anti-PD-1/PD-L1 naïve, defined as never having previously been treated with a drug that targets PD-1.

16. The method of claim 1, wherein the subject has head and neck squamous cell carcinoma (HNSCC).

17. The method of claim 16, wherein the subject has, or is selected on the basis of, at least one of the following attributes: (a) has advanced or metastatic disease; (b) is not a candidate for curative surgery or curative radiation; (c) has PD-L1 expression of CPS ≥1% by a local IHC assay; (d) has received no prior systemic treatment for recurrent or metastatic HNSCC; or (e) is anti-PD-1/PD-L1 naïve, defined as never having previously been treated with a drug that targets PD-1.

18. The method of claim 1, wherein the bispecific EGFRxCD28 antibody or antigen-binding fragment thereof is administered at a dose of about 0.1 mg to about 3000 mg.

19. The method of claim 1, wherein the bispecific EGFRxCD28 antibody or antigen-binding fragment thereof is administered at a dose of about 0.01 mg, 0.03 mg, 0.05 mg, 0.1 mg, 0.3 mg, 0.5 mg, 1 mg, 2 mg, 3 mg, 4 mg, 5 mg, 6 mg, 8 mg, 10 mg, 15 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1000 mg, 1200 mg, 1500 mg, 1800 mg, 2000 mg, 2400 mg, 2700 mg, or 3000 mg.

20. The method of claim 1, wherein the anti-PD-1 antibody or antigen-binding fragment thereof is administered at a dose of about 50 mg to about 1500 mg.

21. The method of claim 1, wherein the anti-PD-1 antibody is administered at a dose of 350 mg.

22. The method of claim 1, wherein the method comprises administering one or more doses of the bispecific EGFRxCD28 antibody or antigen-binding fragment thereof in combination with one or more doses of the anti-PD-1 antibody or antigen-binding fragment thereof.

23. The method of claim 22, wherein each of the one or more doses of the bispecific EGFRxCD28 antibody or antigen-binding fragment thereof is about 0.1 mg to about 3000 mg.

24. The method of claim 23, wherein each of the one or more doses is about 0.01 mg, 0.03 mg, 0.05 mg, 0.1 mg, 0.3 mg, 0.5 mg, 1 mg, 2 mg, 3 mg, 4 mg, 5 mg, 6 mg, 8 mg, 10 mg, 15 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1000 mg, 1200 mg, 1500 mg, 1800 mg, 2000 mg, 2400 mg, 2700 mg, or 3000 mg.

25. The method of claim 22, wherein each of the one or more doses of the anti-PD-1 antibody or antigen-binding fragment thereof is about 50 mg to about 1500 mg.

26. The method of claim 22, wherein each of the one or more doses of the anti-PD-1 antibody is 350 mg.

27. The method of claim 22, wherein each of the one or more doses of the bispecific EGFRxCD28 antibody or antigen-binding fragment thereof and/or the one or more doses of the anti-PD-1 antibody or antigen-binding fragment thereof is administered 0.5 to 14 weeks after the immediately preceding dose.

28. The method of claim 22, wherein each of the one or more doses of the bispecific EGFRxCD28 antibody or antigen-binding fragment thereof and/or the one or more doses of the anti-PD-1 antibody or antigen-binding fragment thereof is administered once every week, once every two weeks, once every three weeks, once every four weeks, once every five weeks or once every six weeks.

29. The method of claim 22, wherein each dose of the bispecific EGFRxCD28 antibody or antigen-binding fragment thereof is administered once every week.

30. The method of claim 22, wherein each of the one or more doses of the bispecific EGFRxCD28 antibody or antigen-binding fragment thereof is administered once every three weeks.

31. The method of claim 22, wherein each of the one or more doses of the anti-PD-1 antibody or antigen-binding fragment thereof is administered once every three weeks.

32. The method of claim 1, wherein the bispecific EGFRxCD28 antibody or antigen-binding fragment thereof and/or the anti-PD-1 antibody or antigen-binding fragment thereof are administered intravenously.

33. The method of claim 1, wherein the bispecific EGFRxCD28 antibody or antigen-binding fragment thereof and/or the anti-PD-1 antibody or antigen-binding fragment thereof are administered subcutaneously.

34. The method of claim 1, wherein the bispecific EGFRxCD28 antibody or antigen-binding fragment thereof and the anti-PD-1 antibody or antigen-binding fragment thereof are administered on the same day.

35. The method of claim 1, wherein the bispecific EGFRxCD28 antibody or antigen-binding fragment thereof and the anti-PD-1 antibody or antigen-binding fragment thereof are administered on different days.

36. The method of claim 35, wherein the bispecific EGFRxCD28 antibody or antigen-binding fragment thereof is administered before or after the anti-PD-1 antibody or antigen-binding fragment thereof.

37. The method of claim 1, comprising the steps of:
(i) administering to the subject the bispecific EGFRxCD28 antibody or antigen-binding fragment thereof at a dose of 0.1 mg to 3000 mg subcutaneously or intravenously once every week or once every 3 weeks for a period of monotherapy, wherein the period of monotherapy is at least 3 weeks; and
(ii) administering to the subject the bispecific EGFRxCD28 antibody or antigen-binding fragment thereof at a dose of 0.1 mg to 3000 mg subcutaneously or intravenously once every week or once every 3 weeks, and administering to the subject an anti-PD-1 antibody or antigen-binding fragment thereof at a dose of 150 mg to 500 mg intravenously or subcutaneously once every 3 weeks.

38. The method of claim 37, wherein the period of monotherapy is at least 3 weeks, at least 4 weeks, at least 5 weeks, or at least 6 weeks.

39. The method of claim 37, wherein, during step (ii), the anti-PD-1 antibody or antigen-binding fragment thereof is administered on a different day as the bispecific EGFRxCD28 antibody or antigen-binding fragment thereof.

40. The method of claim 37, wherein, during step (ii), the anti-PD-1 antibody or antigen-binding fragment thereof is administered on the same day as the bispecific EGFRxCD28 antibody or antigen-binding fragment thereof.

41. The method of claim 1, further comprising administering to the subject one or more additional agents to treat one or more symptoms of an immune-related adverse event.

42. The method of claim 41, wherein the one or more additional agents comprise an IL-6 receptor inhibitor, a corticosteroid, and/or a non-steroidal anti-inflammatory drug (NSAID).

43. The method of claim 1, wherein the subject has stable disease, partial response, or complete response upon administration of the bispecific antibody or antigen-binding fragment thereof for at least one week at a dose of about 0.1 mg to about 3000 mg in combination with the anti-PD-1 antibody or antigen-binding fragment thereof.

44. The method of claim 1, wherein the anti-PD-1 antibody or antigen-binding fragment thereof is cemiplimab, nivolumab, pembrolizumab, MEDI0608, BI 754091, spartalizumab (PDR001), camrelizumab (SHR-1210), JNJ-63723283, MCLA-134, toripalimab, sintilimab, tislelizumab, serplulimab, dostarlimab, retifanlimab, zimberelimab, penpulimab, pidilizumab, HX008, balstilimab or ezabenlimab, or an antigen-binding fragment of any of the foregoing.

45. The method of claim 1, wherein the anti-PD-1 antibody or antigen-binding fragment thereof comprises the heavy chain complementarity determining regions (HCDR1, HCDR2 and HCDR3) of a heavy chain variable region (HCVR) comprising the amino acid sequence of SEQ ID NO: 73 and three light chain complementarity determining regions (LCDR1, LCDR2 and LCDR3) of a light chain variable region (LCVR) comprising the amino acid sequence of SEQ ID NO: 74.

46. The method of claim 1, wherein the anti-PD-1 antibody or antigen-binding fragment thereof comprises three HCDRs (HCDR1, HCDR2 and HCDR3) and three LCDRs (LCDR1, LCDR2 and LCDR3), wherein HCDR1 comprises the amino acid sequence of SEQ ID NO: 75; HCDR2 comprises the amino acid sequence of SEQ ID NO: 76;
HCDR3 comprises the amino acid sequence of SEQ ID NO: 77; LCDR1 comprises the amino acid sequence of SEQ ID NO: 78; LCDR2 comprises the amino acid sequence of SEQ ID NO: 79; and
LCDR3 comprises the amino acid sequence of SEQ ID NO: 80.

47. The method of claim 46, wherein the HCVR comprises the amino acid sequence of SEQ ID NO: 73 and the LCVR comprises the amino acid sequence of SEQ ID NO: 74.

48. The method of claim 45, wherein the anti-PD-1 antibody comprises a heavy chain comprising the amino acid sequence of SEQ ID NO: 81 and a light chain comprising the amino acid sequence of SEQ ID NO: 82.

49. The method of claim 1, wherein the anti-PD-1 antibody is cemiplimab, or an antigen-binding fragment thereof.

50. The method of claim 1, wherein the first antigen-binding domain that binds CD28 comprises three heavy chain complementarity determining regions (CDR-H1, CDR-H2 and CDR-H3) contained within a heavy chain variable region (HCVR) comprising the amino acid sequence of SEQ ID NO: 10; and three light chain complementarity determining regions (CDR-L1, CDR-L2 and CDR-L3) contained within a light chain variable region (LCVR) comprising the amino acid sequence of SEQ ID NO: 16.

51. The method of claim 50, wherein CDR-H1 comprises the amino acid sequence:
GGSISSYY (SEQ ID NO: 12), CDR-H2 comprises the amino acid sequence: IYYSGIT (SEQ ID NO: 6), and CDR-H3 comprises the amino acid sequence: ARWGVRRDYYYYGMDV (SEQ ID NO: 14).

52. The method of claim 50, wherein CDR-L1 comprises the amino acid sequence:
QSVSSSY (SEQ ID NO: 18), CDR-L2 comprises the amino acid sequence: GAS (SEQ ID NO: 20), and CDR-L3 comprises the amino acid sequence: QQYGSSPWT (SEQ ID NO: 22).

53. The method of claim 50, wherein the first antigen-binding domain comprises a HCVR comprising the amino acid sequence of SEQ ID NO:
10, and a LCVR comprising the amino acid sequence of SEQ ID NO: 16.

54. The method of claim 1, wherein the second antigen binding domain that binds human EGFR comprises three heavy chain complementarity determining regions (CDR-H1, CDR-H2 and CDR-H3) contained within a heavy chain variable region (HCVR) comprising the amino acid sequence of SEQ ID NO: 2; and three light chain complementarity determining regions (CDR-L1, CDR-L2 and CDR-L3) contained within a light chain variable region (LCVR) comprising the amino acid sequence of SEQ ID NO: 16.

55. The method of claim 54, wherein CDR-H1 comprises the amino acid sequence: GDSIITFY (SEQ ID NO: 4), CDR-H2 comprises the amino acid sequence: IYYSGIT (SEQ ID NO: 6), and CDR-H3 comprises the amino acid sequence: ARVSEDSYFHYGMDV (SEQ ID NO: 8).

56. The method of claim 54, wherein CDR-L1 comprises the amino acid sequence: QSVSSSY (SEQ ID NO: 18); CDR-L2 comprises the amino acid sequence: GAS (SEQ ID NO: 20); and CDR-L3 comprises the amino acid sequence: QQYGSSPWT (SEQ ID NO: 22).

57. The method of claim 54, wherein the second antigen-binding domain comprises a HCVR comprising the amino acid sequence of SEQ ID NO: 2, and a LCVR comprising the amino acid sequence of SEQ ID NO: 16.

58. The method of claim 1, wherein:
(a) the first antigen binding domain that binds human CD28 comprises a heavy chain variable region that comprises the amino acid sequence set forth in SEQ ID NO: 10, and a light chain variable region that comprises the amino acid sequence set forth in SEQ ID NO: 16; and
(b) the second antigen binding domain that binds human EGFR comprises a heavy chain variable region that comprises the amino acid sequence set forth in SEQ ID NO: 2; and a light chain variable region that comprises the amino acid sequence set forth in SEQ ID NO: 16.

59. The method of claim 50, wherein the bispecific antibody comprises a first heavy chain comprising the amino acid sequence of SEQ ID NO: 26.

60. The method of claim 50, wherein the bispecific antibody comprises a second heavy chain comprising the amino acid sequence of SEQ ID NO: 24.

61. The method of claim 50, wherein the bispecific antibody comprises a light chain comprising the amino acid sequence of SEQ ID NO: 28.

62. The method of claim 50, wherein the bispecific antibody comprises a first heavy chain comprising the amino acid sequence of SEQ ID NO: 26, a second heavy chain comprising the amino acid sequence of SEQ ID NO: 24, and a common light chain comprising the amino acid sequence of SEQ ID NO: 28.

63. The method of claim 50, wherein the first antigen-binding domain comprises a heavy chain comprising the amino acid sequence of SEQ ID NO: 26 and a light chain comprising the amino acid sequence of SEQ ID NO: 28.

64. The method of claim 50, wherein the second antigen-binding domain comprises a heavy chain comprising the amino acid sequence of SEQ ID NO: 24 and a light chain comprising the amino acid sequence of SEQ ID NO: 28.

65. The method of claim 1, wherein the bispecific EGFRxCD28 antibody is REGN7075, or an antigen-binding fragment thereof.

66. The method of claim 1, further comprising administering chemotherapy, optionally platinum-based chemotherapy to the subject.
